(19) United States
(12) Patent Application Publication
(10) Pub. No.: US 2021/0369677 A1
(43) Pub. Date: Dec. 2, 2021

(54) SMALL MOLECULE ACTIVATORS OF POLYCYSTIN-2 (PKD2) AND USES THEREOF

(71) Applicant: Northwestern University, Evanston, IL (US)

(72) Inventor: Paul G. DeCaen, Wilmette, IL (US)

(21) Appl. No.: 17/303,397

(22) Filed: May 27, 2021

Related U.S. Application Data
(60) Provisional application No. 63/030,732, filed on May 27, 2020.

(51) Int. Cl. A61K 31/4174; A61K 31/145; A61K 31/17

(52) U.S. Cl. CPC A61K 31/4174; A61K 31/17; A61K 31/145

(57) ABSTRACT

Disclosed are methods of treating diseases or disorders associated with the activity of polycyctic kidney disease 2 (PKD2). The disclosed methods may be utilized to treat diseases or disorders associated with polycystic kidney disease, for example autosomal dominant polycystic kidney disease (ADPKD). Also disclosed are activators of PKD2. The disclosed compounds may also be used in pharmaceutical compositions and methods for treatment of polycystic kidney disease or disorders associated with PKD2 activity.

### CROSS-REFERENCE TO RELATED PATENT APPLICATIONS

The present application claims the benefit of priority under 35 U.S.C. § 119(e) to U.S. Provisional Application No. 63/030,732, filed on May 27, 2020, the content of which is incorporated herein by reference in its entirety.

### STATEMENT REGARDING FEDERALLY SPONSORED RESEARCH OR DEVELOPMENT

This invention was made with government support under DK123463 awarded by the National Institutes of Health. The government has certain rights in the invention.

### SEQUENCE LISTING

A Sequence Listing accompanies this application and is submitted as an ASCII text file of the sequence listing named “702581_1928_ST25.txt” which is 33 KB in size and was created on May 27, 2021. The sequence listing is electronically submitted via EFS-Web with the application and is incorporated herein by reference in its entirety.

### FIELD

The field of the invention relates to small molecule activators of polycytin-2 (PKD2) and the use thereof in treating diseases and disorders associated with PKD2 biological activity. In particular, the invention relates to small molecule activators of polycystin-2 (PKD2) useful for treating autosomal dominant polycystic kidney disease (ADPKD).

### BACKGROUND

Autosomal dominant polycystic kidney disease (ADPKD) is a potentially lethal, common monogenetic disorder for which there is no drug cure. Mutations in polycystin-2 (PKD2) cause 15% of all cases of ADPKD and affect ˜105,000 people in the United States and more world-wide. PKD2 is a voltage dependent channel which is localized to primary cilia in the kidney. ADPKD-causing variants in PKD2 retain localization to the cilia in patient-derived kidney cells but do not properly conduct ions. Therefore, there is a need in the art for therapeutic agents that can activate PKD2 and restore proper conduction of ions, for example, in patients having ADPKD-causing variants. Here, the inventors disclose methods for identifying activators of PKD2 and small molecules identified by the disclosed methods. The identified small molecules may be useful for formulating therapeutic agents for treating diseases and disorders associated with PKD2 activity, such as ADPKD.

### SUMMARY

Disclosed are compounds, pharmaceutical compositions comprising the compounds, and methods of using the compounds and pharmaceutical compositions for treating a subject having or at risk for developing a disease or disorder associated with polycystin 2 (PKD2) biological activity. The disclosed compounds may activate the biological activity of PKD2. As such, the disclosed compounds and pharmaceutical compositions may be utilized in methods for treating a subject having or at risk for developing a disease or disorder that is associated with PKD2 activity which may be autosomal dominant polycystic kidney disease (ADPKD).

In some embodiments, the disclosed methods include treating and/or preventing a disease or disorder associated with polycystin 2 (PKD2) activity in a subject in need thereof. In the disclosed methods, the subject may be administered an effective amount of a therapeutic agent that activates the biological activity of PDK2.

Also disclosed are methods for identifying agents that activate the biological activity of PKD2. The methods may be performed in order to identify therapeutic agents for treating and/or preventing diseases and disorders that are associated with the biological activity of PKD2, such as ADPKD, in a subject in need thereof.

### BRIEF DESCRIPTION OF THE DRAWINGS

FIG. 1. The atomic interactions disrupted by TOP domain finger 1 motif variants and their impact of cilia localization. (A) The location of the TOP domain (blue) structure of the polycystin-2 channel (PDB: 5T4D). Expanded view of the interactions made by three pathogenic variant sites (underlined) within the finger 1 motif. B) Super resolution SIM (structured illumination microscopy, Scale bar=3 μm) images of HEKPKD2^{Null} cells stably expressing variants of PKD2-GFP immunolabeled with anti-ARL13B antibody. C) Cilia fluorescence colocalization analysis using the Pearson's correlation coefficient. D) Comparison of cilia length from the indicated HEK cell lines. Student's t-test results (see methods) comparing WT and variants channels (N.S., p>0.05). The number cells tested are indicated in the parenthesis and error bars are equal to standard deviation.

FIG. 2. Finger 1 variants cause of loss of channel function in the primary cilia. A) Image of a voltage-clamped primary cilia from a HEK PKD2^{Null} cell stably expressing WT PKD2-GFP. Note that the GFP signal is present in the primary cilium and that the patch electrode is sealed onto cilia membrane. Scale bar=5 μM. B) Left, Exemplar ciliary WT polycystin-2 currents activated by voltage ramps and blocked by 10 μM lanthanum (La³⁺) or gadolinium (Gd³⁺). Right, the time course of outward (100 mV) and inward (−100 mV) current block by trivalent ions. C) Lack of ciliary current measured from HEK cells expressing TOP domain variants. D) Box (mean S.E.M.) and whisker (mean±S.D.) plots of cilia total outward (100 mV) and inward cilia (−100 mV) current measured from HEK cilia expressing the indicated channels. Number of cilia for each channel is indicated in parenthesis. Resulting P values<0.05 from Student's T-test values comparing the outward currents from WT and variants are indicated by an asterisk.

FIG. 3. Finger 1 variants shift voltage-dependent opening of polycystin-2. A) Exemplar single channel recordings measured from the cilia of HEK PKD2^{Null} cells expressing WT or C331S polycystin-2 channels. B) Average channel open probability plotted as a function of voltage. The data was fit to a Boltzmann equation to estimate the half maximal voltage response (V_{1/2}) and the slope factor (Z), which was used to calculate the free energy required to open the channels (ΔG°, see methods for equation). C) Average single channel current amplitudes. Conductance (γ) estimated by fitting the average single channel currents to a linear equation. Error bars indicate S.D. from 6 cilia recordings from each cell type.

FIG. 4. The finger 1 disulfide bond is required for polycystin-2 and polycystin-2L1 gating. (A-D) Left, exemplar currents activated by voltage ramps measured from the primary cilia of HEK cells stably expressing the indicated polycystin-2 channels under control and reducing conditions. Right, the time course of inward (−100 mV) and outward (100 mV) current block by reducing agents and gadolinium. E) Bar graph showing the average percent block of outward polycystin-2 currents by TCEP (green) and GSH (purple). N=5 cells and error bars are equal to S.D. F) Left, exemplar whole cell polycystin-2L1 currents activated by voltage ramps and their block by reducing agents. Right, the time course of current block. G) Bar graph showing the average percent block of outward polycystin-2L1 currents by the reducing agents. N=6 cells and error is S.D.

FIG. 5. The C331S variant destabilizes the TOP domain without impairing channel assembly. The 3.4 Å cryo-EM structure of the polycystin-2 C331S variant. A) Transmembrane (left) and extracellular (right) views of the superimposed wild type (blue) and C331S (orange) polycystin-2 structures. Inset, an expanded view of the finger 1 cysteine interactions. B) The pore domain (S5 and S6) from two subunits of the wild type and variant channel structures. Note that there is little difference in the pore domain structures. C-E) Size exclusion chromatography profiles of the wild type channels under neutral and reducing conditions (10 mM GSH); and for the C331S mutant with samples treated at indicated temperatures. F) Thermal denature profiles for WT channels under standard and reducing conditions, and for the C331S variant. The C331S and WT channels treated with GSH denature at lower temperature compared to the wild type channel in neutral conditions.

FIG. 6. The conservation of the TOP domain within polycystins. A) An alignment of the TOP domain using the ClustalW multiple sequence alignment program applying the default color scheme with <80% conservation of character: Hydrophobic; polar; glutamine, glutamate, aspartate are designated. Special amino acids also are designated: glycine; proline and tyrosine or histidine. The barrels indicate alpha helices and arrows indicate beta-sheet turns found in the polycystin-2 structure. The finger 1 motif is indicated and the variant sites are underlined. B) Sequencing results aligned from the parental HEK and PKD2^{Null} cell lines showing the shift in the reading frame and premature truncation site introduced by the CRISPR/Cas9 method.

FIG. 7. Primary cilia and ER localization of polycystin-2. A) Super resolution images of HEK PKD2^{Null} cells stably expressing of PKD2-GFP and immunolabeled with antibodies that target primary cilia epitopes (ARL13B, aceylated tubulin, adenylate cyclase) or with ER-tracker. B) Fluorescence colocalization analysis using the Pearson's correlation coefficient. C) Cilia localization of R322Q, R324Q TOP domain variants as they relate to FIG. 1. Part numbers and concentrations of the antibodies and reagents used are listed in Table 2.

FIG. 8. Genetic ablation or overexpression of PKD2 does not alter calcium release from intracellular stores. A) Confocal images of colocalized PKD2-GFP in the cilia (ARL13B) and endoplasmic reticulum (ER-tracker) ofPKD2^{Null} cells. B) Images of parental HEK cells expressing the mCherry vector and fura-2 emission ratio (240k: 280k) before and after 50 μM carbachol (CCh) stimulation. C) Average time course the fura-2 emission ratio from the indicated cells expressing PKD2 variants. Grey lines indicate the responses from individual cells and the black circles indicate the average response from the indicated trials (error±SD). D) The average intracellular calcium concentration ([Ca²⁺]) measured before (resting) and after CChl-induced calcium release for each HEK cell genotype.

FIG. 9. Expression of PKD2 does not alter calcium release from HEK cells expressing endogenous MIR or over-expressing MIR-cherry. A) Average time course pf the fura-2 emission ratio from HEK expressing PKD2 genotypes (N=trials, error±S.D.). Top, activation of intracellular calcium Fura-2 emission by 3 μM carbachol (CCh) from HEK cells expressing the endogenous muscarinic 1 receptor (MIR). Bottom, Fura-2 emissions from HEK cells overexpressing M1R-mcherry. B) The resulting average intracellular calcium concentration ([Ca²⁺ᵢₙ) measured before and after CCh stimulation.

FIG. 10. Single channel open events measured from cilia expressing finger 1 variants (R325P, R325Q, R323Q and R323W). Exemplar single channel recordings from the cilia of HEK PKD2^{Null} cells expressing the indicated variant channels, as it relates to the results shown in FIG. 3.

FIG. 11. Mutations in conserved finger 1 interaction sites cause a loss of polycystin-2L1 function. A) Structural overlay of the polycystin-2 (5T4D) and polycystin-2L1 (5Z1W) channels with expanded views of the conserved molecular interactions at the finger 1 variant sites^{15,31}. B) Left, exemplar polycystin-2L1 currents recorded from the plasma membrane of HEK cells and blocked by 10 μM lanthanum (La³⁺) or gadolinium (Gd³⁺). Whole cell currents were activated by 400 ms voltage ramps from −100 to 100 mV. Right, the time course of outward (100 mV) and inward (−100 mV) current block by trivalent ions. C) Whole cell currents measured from an transfected HEK cell, D) and those expressing polycystin-2L1 finger 1 mutations analogous to the ADPKD-causing variants found in polycystin-2 E) Box (mean±S.E.M.) and whisker (mean±S.D.) plots of total outward (100 mV) and inward (−100 mV) whole cell current (N=22 cells from each group). Resulting P values <0.05 from Student's t-test values comparing the outward currents from WT and mutations are indicated by an asterisk.

FIG. 12. Structure determination of the polycystin-2 C331S channel. A) A representative micrograph shows polycystin-2 C331S channel particles recorded on a Krios microscope. B) 2D classes of the C331S channel calculated with RELION show that the overall tetrameric architecture is preserved. C) FSC curves for resolution estimation and model validation. FSC curves reported by RELION without (unmasked, blue) or with (masked, black) a mask to flatten regions outside protein density prior to FSC calculation. FSC curve (model vs summed half map, purple) shows overall agreement between model and map. D) Local resolution determined by RELION. E) Angular distribution plot of all particle projections. Cylinder heights are proportional to number of particles assigned to each set of Euler angles.

FIG. 13. Reconstruction of the polycystin-2 C331S channel. Flow chart of image processing for the polycystin-2 C331S channel. 3D classification with k=4 yielded one class of ˜74 k particles, which were auto-refined to 3.24 Å resolution with C4 symmetry imposed. Another 2 classes (total ˜174 k particles) showed visible disordered TOP domain and refinement of pooled particles in these two classes with or without C4 symmetry yielded two similar maps.

FIG. 14. EM density maps of various regions of the C331S channel. The maps are sharpened with a b factor of −100 Å2. In panel A, the finger 1 region that harbors the C331S mutation is highlighted.

FIG. 15. Cryo-EM collection and refinement data for the C331S variant structure.

FIG. 16. Concentration and part numbers of the antibodies used for cilia super-resolution colocalization analysis.

FIG. 17. Disruption of Ca² affinity for the polycystin-2 EF hand by single alanine substitutions and mouse model deletion and double mutations. A) Structural alignment of two polycystin-2 channel structures solved using cryo-EM (PDBs: 5MKE in grey and 5T4D in light blue)(Shen et al., 2016; Wilkes et al., 2017). The hypothetical location of the unresolved internal CTD, which contains coiled-coil and EF hand motifs, is represented by a grey density. Inset, the human polycystin-2 EF hand structure solved by solution NMR (PDB: 2Y4Q, residues 721-793) with the Ca²⁺-coordinating vertices highlighted (Allen et al., 2014). B) Top, an alignment of the human and mouse polycystin-2 EF hands. The locations of ADPKD-associated truncating frame shift variants are indicated by blue arrows. The location of the mouse model deletion (del-Z) and double alanine substitution (−X-Z) mutations used in this study are indicated in black italics. Bottom, calorimetry results measuring the differences in Ca²⁺-EF hand affinity when the coordinating vertices are substituted for alanine in the human CTD fragment (704-797). Average calculated Ca²⁺ affinity (Kd) from six replicates per peptide are graphed (Error=S.D.). Angled bar graph caps indicated that no affinity was detected from mutant EF hand fragments when tested up to 2 mM calcium.

FIG. 18. Intraciliary Ca²⁺ activates primary cilia currents conducted by WT and −X-Z polycystin-2 channels with similar potency. A) Establishing high resistance seals with primary cilia in the inside out configuration. Left, scanning EM images of a cilia patch electrode before (top) and after (bottom) fire polishing. Middle, images of a voltage clamped primary cilia from an HEK cell expressing PKD2-GFP. Note green fluorescence signal illuminates the cilia and intracellular compartments. Right, the tip of the cilia is lifted and torn from the cell to establish the inside-out configuration, where intraciliary Ca²⁺ was exchanged in the perfusate. B) Exemplar WT (left) or −X-Z (right) human polycystin-2 single channel currents recorded under increasing intraciliary Ca²⁺ while voltage clamped at 50 mV. Bottom, stochastic open channel events from the expanded time scale of the 100 μM calcium condition. C) The relationship of intraciliary Ca²⁺ and normalized integrated current from WT and −X-Z channels (N=8 cilia, Error=S.D.)

FIG. 19. Ca²⁺-dependent modulation (CDM) of human polycystin-2 is unchanged after abolishing Ca²⁺-EF hand affinity using the −X-Z mutations. A) Exemplar open channel events captured from inside-out patches with one channel in the cilia membrane. B, C) Average channel open probability (Po) plotted as a function of voltage in the presence of 100 nM and 100 μm intracellular calcium. The data was fit the Boltzmann equation to estimate the half maximal voltage response (V_{1/2}). Error bars indicate standard deviation from 8 cilia recordings from each channel type.

FIG. 20. Pkd2^{-X-Z/-X-Z} mice do not develop polycystic kidney or polycystic liver disease. A) Immunoblotting with anti-V5 antibody showing absence of epitope tagged Pkd2 signal in wild type kidney (inverted triangle), and dose-dependent expression of Pkd2-V5 in Pkd2^{-X-Z/+} (square) and Pkd2^{-X-Z/-X-Z} (triangle) kidney lysates. B) Two kidney-to-body weight ratio for 9 month old wild type (red inverted triangle), Pkd2^{-X-Z/+} (square), Pkd2^{-X-Z/-X-Z} (triangle) and Pkd2^{-X-Z/null} (circle) mice. The P value resulting from a one-way ANOVA statistical analysis is shown above the plot. The number animals per genotype used are indicated within the parenthesis. C) Representative kidney scans and D) hematoxylin and eosin staining of kidney sections (top row) and periportal liver sections (bottom row) from 9 month old mice with the indicated (color coded) genotypes. Arrows indicate normal bile ducts: v, periportal vein. Scale bars: 100 μm kidney panels (top); 25 μm liver panels (lower).

FIG. 21. Pkd2^{del-Z/del-Z} mice do not develop polycystic kidney disease. A) Exemplar MRI images of the frontal (top) and transverse (bottom) plane of the thoracic cavity of 12 month old mice expressing the indicated Pkd2 genotypes. Kidney and liver cysts are visible in both planes from conditional cPkd2 knock out animals (Pax8^{rTA}; TetO-cre; Pkd2^{fl/fl}) six months after induction with doxycycline, as previously described (Liu et al., 2018; Ma et al., 2013). However, no obvious cysts were detected from mice expressing constitutive Pkd2^{del-Z} alleles B) Top, exemplar black and white images of hematoxylin and eosin stained, medial kidney sections. Bottom, FIJI analysis results used to identify cystoid foramen (blue) after reverse-negative processing the above images (Schindelin et al., 2012). C) Box (S.E.M.) and whisker (S.D.) plots of the average diameter (left) and number (right) of foramen from kidney sections from 12 month old mice (N=6 mice per genotype). P values resulting from a one-way ANOVA are shown above the plots.

FIG. 22. Gα_{q}-mediated Ca²⁺ release from ER stores are the same in collecting duct cells isolated from WT and Pkd2^{del-Z/del-Z} mice. A) An exemplar Ca²⁺ dependent fura-2 emission ratios (340λ:380λ) from primary cultures of pIMCD cells isolated from mice Pkd2^{+/+} mice. B, C) Average cytosolic fura-2 emission ratios from pIMCD cells isolated from Pkd2^{+/+} and Pkd2^{del-Z/del-Z} mice before and after Gα_{q}-mediated Ca²⁺ release using B) 800 nM arginine vasopressin (AVP) or C) 50 μM carbachol (CCh). The number trials (N) and cells tested listed per test group. Student's t-test P-value are indicated above the bar graphs (error±SD). P values resulting from Student's t test analysis are shown above the graphs.

FIG. 23. Isothermal calorimetry results measuring Ca2+-EF hand affinity (Kd) of Polycystin-2. Tabulated average and standard deviation of calcium affinity results derived from the calorimetry experiments described in FIG. 17 and FIG. 25.

FIG. 24. Cilia electrophysiology results measuring Ca2+ potency for polycystin-2 activation (EC50) and CDM of voltage dependent gating (V1/2). Tabulated average EC₅₀ and V_{1/2} values derived from FIGS. 18, 19 and FIGS. 27, 28.

FIG. 25. Calorimetric titration of divalent ions into the CTD of polycystin-2. A)-D) ITC profiles corresponding to calcium binding to the wild type or mutant polycystin CTD (704-797). The upper panel of each profile shows the raw data of the heat changes upon successive injection of 1 μL CaCl₂), performed at 25 deg. C. The bottom panel shows the binding curve where the peaks of the heat change were integrated and plotted against the molar ratio of accumulated calcium to the concentration of the protein fragment. The line represents a non-linear fit based on a single-site binding model. The stoichiometry of Ca2+ to WT CTD peptide (N)=0.75±0.06: the change in enthalpy (ΔH)=−17.7±1.5 KJ/mol and entropy (ΔS)−40.3±5.3 kcal/mol·K; Error=SEM. E) Exemplar profiles demonstrate the lack of Zn2+ and Mg2+ affinity for the polycystin-2 CTD. Note the difference in time scales between the data sets shown in A-D and E. In some cases, the binding curves were expanded in the inset for better visualization

FIG. 26. Ca²⁺-dependent desensitization (CDD) of human and mouse polycystin-2 channels. A) Left, desensitization of whole cilia currents measured from HEK cells expressing WT or −X-Z EF hand PKD2 mutant channels under high intracellular free calcium (30 μM) captured at minute time intervals (1′, 2′ and 3′). Right, corresponding time courses of desensitization plotted from individual cilia patches (open symbols) and the average responses (filled symbols). B) Same as in A, but measuring CDD from pIMCD cells isolated from Pkd2++ or Pkd2del-Z del-Z mice. C, D) Box (S.E.M.) and whisker (S.D.) plots comparing peak current magnitudes measured from A and B at two and three minute intervals. Error is equal to S.D., N=7 cilia for each genotype. P values resulting from Student's t test analysis are shown above the graphs.

FIG. 27. Ca2+ equally activates primary cilia currents from mice expressing WT and EF hand mutant Pkd2^{del-Z} alleles. A) Polycystin-2 single channels recorded from primary cilia of collecting duct cells isolated from mice expressing WT and Pkd2del-z alleles. Using the same protocol described in FIG. 2, polycystin-2 channel open events were captured in the inside-out configuration and intraciliary calcium was raised in the perfusate. The traces within the dashed square show the expanded time scale in the 100 μM calcium condition. B) The relationship of ciliary calcium and normalized integrated current from genotypes (N=7 cilia, Error=S.D.).

FIG. 28. CDM of ciliary polycystin-2 is similar from mice expressing WT and Pkd2^{del-Z} alleles. A) Exemplar polycystin-2 single channel recordings measured in the inside-out cilia patch configuration. pIMCD cells were isolated from the kidney medulla of mice expressing WT and del-Z alleles that co-expressed the ARL13B-GFP transgene. B, C) Average channel open probability plotted as a function of voltage in the presence of 100 nM and 100 μM intracellular calcium. The data was fit the Boltzmann equation to estimate the half-maximal voltage response (V_{1/2}). Error bars indicate S.D. from 6 cilia recordings from each cell type.

FIG. 29. Structure of PKD2 and model of exemplary compound (NS1643) interacting with PKD2 to activates transport of Ca²⁺.

FIG. 30. Shift of curve of Channel Open probability (Po) at a given voltage in the presence of a drug (Rx) that activates PKD2 channel.

FIG. 31. A three stage testing strategy identifies NS1643 as an exemplary PKD2 activator. Top, the screening of each stage. A) Stage 1 (Yeast Growth): P. pastoris (strain Y5634) growth is dependent on induction and stimulation of PKD2 cation transport by NS1643 (EC₅₀=1.1 μM). B,C) Stage 2 (Cilia Ca²⁺ Fluorescence and Membrane Potential): Measuring PKD2 activation by NS1643 using increased ciliary Ca²⁺ fluorescence (Right, smo-GCaMP3, EC₅₀=2.3 μM) and membrane potential (Left, Smo-ArcLight; EC₅₀=μm) from human kidney collecting duct cells. Bottom, cilia Ca²⁺ responses from cells with PKD2 present and knocked out using CRISPR=Cas9 (N=17 cells each). D, E) Stage 3 (Cilia Electrophysiology): image of a patched cilia from collecting ducts and corresponding PKD2-dependent current activated by NS1643.

FIG. 32. Effectiveness of NS1643 as a PKD2 activator in the three screening stages of FIG. 31.

FIG. 33. Thermal stability of PKD2 and C331S in the presence of NS1643 suggests that that NS1643 stabilizes PKD2 structure.

FIG. 34. Rosetta modeling suggests that NS1643 binds to the TOP domain of PKD2.

FIG. 35. Conductance of PKD2 in the presence of test compounds CAS Number 57265-65-3 (calmidazolium (CMZ)), CAS Number 617-27-0 (W-13), and CAS Number 448895-37-2 (NS1643)

FIG. 36. Model screening method for identifying activators of PKD2.

### DETAILED DESCRIPTION

Current pharmacological treatment for ADPKD uses vasopressin receptor agonist to promote water resorption in the kidney. However, this strategy does not directly address the root-cause of ADPKD, mutated PDK2 protein, and is approved to treat ADPKD simply because it is effective at increasing time to dialysis or transplant in individuals suffering from ADPKD. Furthermore, vasopressin receptor agonist treatment does not show efficacy in reducing kidney volume over 5 years of use.

The polycystin pharmacophore remains outstanding because these channels localize to the primary cilium—an antenna-like organelle that requires innovative tools to study. To address this knowledge gap, our laboratory has established a series of cilia-specific channel activity reporters (voltage and calcium reporters) and model systems (poly cystin-dependent yeast growth) which will be used in a three-stage drug screen to characterize the pharmacology of polycystins. We screened a commercially available chemical library containing 384 compounds for activity against wild type polycystins, and identified three channel activators. PKD2 channel activators may be used to prevent or treat ADPKD and may lead to attenuation of cyst progression in other forms of polycystic kidney disease.

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, autosomal dominant polycystic kidney disease (ADPKD) refers to a multi-systemic and progressive disorder characterized by cyst formation and enlargement in the kidney and other organs (e.g., liver, pancreas, spleen).

A “subject in need thereof” as utilized herein may refer to a subject in need of treatment and/or prevention of a disease or disorder associated with polycystin-2 (PKD2) activity. A subject in need thereof may include a subject having ADPKD characterized by the loss of activity of PKD2. A subject in need thereof may include a subject having a ADKPD that is treated by administering a therapeutic agent that activates the biological activity of PDK2, and/or that inhibits the growth of kidney cysts. A subject in need thereof may include a subject having a mutation in PKD2 that disrupts the biological activity of PKD2 (e.g., a mutation that disrupts activation of the ion transport activity of PKD2). A subject in need thereof may include a subject having a mutation in PKD2 which is a substitution mutation or deletion mutation that disrupts the biological activity of PKD2 (e.g., a mutation that disrupts activation of the ion transport activity of PKD2). A subject in need thereof may include a subject having a mutation in PKD2 characterized as C331S, R332Q, K322W, R325Q, R325P, and combinations thereof.

A “subject in need thereof” as utilized herein may refer to a subject in need of treatment autosomal dominant polycystic kidney disease. In some embodiments, a subject in need thereof may refer to a subject in need of augmenting PKD2 activity.

The term “subject” may be used interchangeably with the terms “individual” and “patient” and includes human and non-human mammalian subjects.

The disclosed compounds, pharmaceutical compositions, and methods may be utilized to treat diseases and disorders associated with PKD2 activity which may include, but are not limited to cell proliferative diseases and diseases and disorders such as autosomal dominant polycystic kidney disease. The disclosed compounds may be utilized to modulate the biological activity of PKD2, including modulating the channel activity of PKD2. The term “modulate” should be interpreted broadly to include “activating” PKD2 biological activity including channel activity.

Polycystin 2 (PKD2) refers to the protein also referred to by the name autosomal dominant polycystic kidney disease type II protein. PKD2 has been shown to have activities that include ion channel activity. The polycystin-2 channel preferentially conducts K and Na⁺ and intraciliary Ca²⁺, enhances its open probability. The compounds disclosed herein may inhibit one or more of the activities of PKD2 accordingly.

Human PKD2 is known to have five isoforms and the disclosed compounds may inhibit one or more activities of isoform 1, isoform 2, isoform 3, isoform 4, and/or isoform 5.

Human PKD2 Isoform 1 has the amino acid sequence in SEQ ID NO:1.

Human PKD2 Isoform 2 has the amino acid sequence in SEQ ID NO:2.

Human PKD2 Isoform 3 has the amino acid sequence in SEQ ID NO: 3.

Human PKD2 Isoform 4 has the amino acid sequence in SEQ ID NO:4.

Human PKD2 Isoform 5 has the amino acid sequence in SEQ ID NO: 5.

Chemical Entities

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C1-C6-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —R¹C(O)N(R²)—, —R¹C(O)N(R²) R³—, —C(O)N R² R³, or —C(O)NH2, wherein R¹, R2 and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that activates the biological activity of polycystin 2 (PKD2) may be administered as a single compound or in combination with another compound that activates the biological activity of PKD2 or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with the biological activity of polycystin 2 (PKD2). As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of polycystin 2 (PKD2).

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Illustrative Embodiments

The following embodiments are illustrative and should not be interpreted to limit the scope of the claimed subject matter.

In some embodiments, the disclosed subject matter relates to methods for treating and/or preventing a disease or disorder associated with polycystin 2 (PDK2) activity in a subject in need thereof, in which the method comprises administering to the subject an effective amount of a therapeutic agent that activates the biological activity of PKD2. Suitable diseases or disorders that may be treated and/or prevented by the disclosed methods may include, but are not limited to, kidney disease. In some embodiments of the disclosed methods, the disease is polycystic disease, and in particular, autosomal dominant polycystic kidney disease (ADPKD). In some embodiments, the disease or disorder is ADPKD characterized by a mutation in polycystin-2 selected from C331S, R322Q, K342W, R325Q, R325P, and combinations thereof (e.g., where C331S, R322Q, K342W, R325Q, R325P are relative to the amino acid sequence of SEQ ID NO:1, 2, 3, 4, or 5).

The therapeutic agent that is administered in the disclosed methods activates the biological activity of PKD2. For example, the therapeutic agent may bind to PKD2 and activate PKD2. In some embodiments of the disclosed methods, the therapeutic agent may bind to PKD2 which results in opening of the PKD2 channel and/or increasing trafficking through the PKD2 channel.

Suitable therapeutic agents may include, but are not limited a therapeutic agent selected from the group consisting of

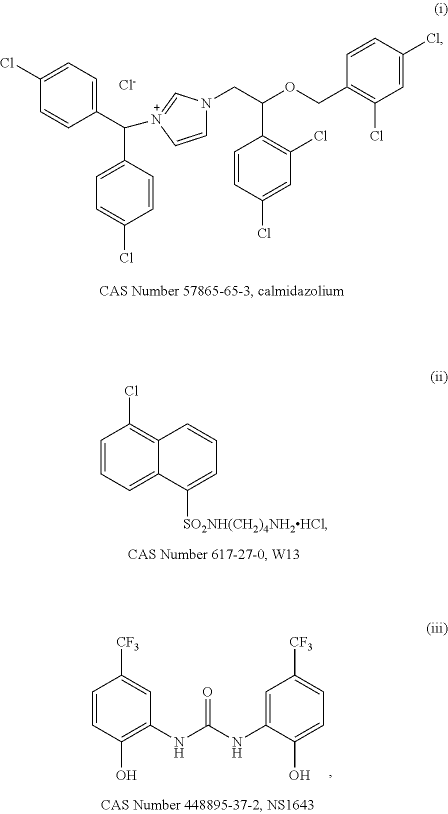

and pharmaceutical salts thereof.

Suitable therapeutic agents may include, but are not limited a therapeutic agent selected from the group consisting of: (i) 1-[bis(4-chlorophenyl)methyl]-3-[2,4-dichloro-o-(2,4-dichlorobenzyloxy) phenethyl]imidazolium (or 1-[bis(4-chlorophenyl)methyl]-3-[2-(2,4-dichlorophenyl)-2-(2,4-dichlorobenzyloxy) ethyl]-1H-imidazolium); (ii) N-(6-Aminohexyl)-5-chloro-1-naphthalenesulfonamide; (iii) 1,3-bis[2-hydroxy-5-(trifluoromethyl)phenyl]urea; and pharmaceutical salts thereof.

In some embodiments of the disclosed methods, the therapeutic agent is:

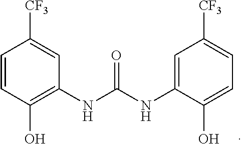 [SMILES: O=C(Nc1cc(C(F)(F)F)ccc1O)Nc1cc(C(F)(F)F)ccc1O]

In some embodiments of the disclosed methods, the therapeutic agent is: 1,3-bis[2-hydroxy-5-(trifluoromethyl)phenyl]urea, or a pharmaceutical salt thereof.

Also disclosed herein are pharmaceutical compositions. The pharmaceutical compositions typically comprise: (a) a therapeutic agent that activates the biological activity of PKD2 as discussed herein; and (b) a pharmaceutical.

In some embodiments, the disclosed pharmaceutical compositions comprise:

(a) a therapeutic agent selected from the group consisting of

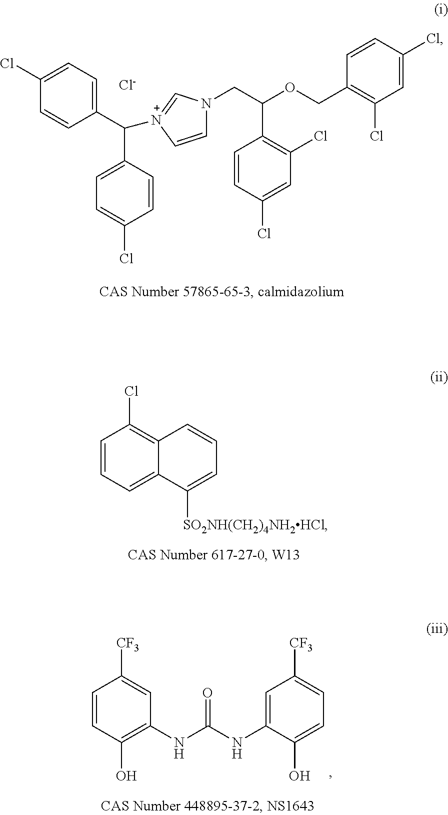

and pharmaceutical salts thereof; and
(b) a suitable pharmaceutical carrier

In some embodiments of the disclosed pharmaceutical compositions, the 10. the therapeutic agent is:

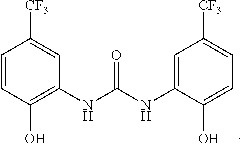 [SMILES: O=C(Nc1cc(C(F)(F)F)ccc1O)Nc1cc(C(F)(F)F)ccc1O]

In some embodiments of the disclosed pharmaceutical compositions, the therapeutic agent is selected from the group consisting of (i) 1-[bis(4-chlorophenyl)methyl]-3-[2,4-dichloro-3-(2,4-dichlorobenzyloxy) phenethyl]imidazolium (or 1-[bis(4-chlorophenyl)methyl]-3-[2-(2,4-dichlorophenyl)-2-(2,4-dichlorobenzyloxy) ethyl]-1H-imidazolium); (ii) N-(6-Aminohexyl)-5-chloro-1-naphthalenesulfonamide; (iii) 1,3-bis[2-hydroxy-5-(trifluoromethyl)phenyl]urea; and pharmaceutical salts thereof.

In some embodiments of the disclosed pharmaceutical compositions, the therapeutic agent is 1,3-bis[2-hydroxy-5-(trifluoromethyl)phenyl]urea, or a pharmaceutical salt thereof.

The disclosed pharmaceutical compositions comprise a therapeutic agent for activating biological activity of PDK2 when administered to a subject in need thereof. As such, the pharmaceutical compositions may comprise an effective amount of the therapeutic agent for activating biological activity of PDK2 when administered to a subject in need thereof.

### Examples

The following Examples are illustrative and should not be interpreted to limit the scope of the claimed subject matter.

### Example 1—Molecular Dysregulation of Ciliary Polycystin-2 Channels Caused by ADPKD-Associated Variants in the TOP Domain

Reference is made to Vien et al., “Molecular dysregulation of ciliary polycystin-2 channels caused by variants in the TOP domain,” Proc. Nat'l Acad. Sci. May 12, 2020, Vol. 117, No. 19, pages 10329-10338, the content of which is incorporated herein by reference in its entirety.

Abstract

Genetic variants in PKD2 which encodes for the polycystin-2 ion channel are responsible for many clinical cases of autosomal dominant polycystic kidney disease (ADPKD). Despite our strong understanding of the genetic basis of ADPKD, we do not know how most variants impact channel function. Polycystin-2 is found in organelle membranes, including the primary cilium-an antennae-like structure on the luminal side of the collecting duct. In this study, we focus on the structural and mechanistic regulation of polycystin-2 by its TOP domain-a site with unknown function that is commonly altered by missense variants. We use direct cilia electrophysiology, cryo-EM and super-resolution imaging to determine that variants of the TOP domain finger 1 motif destabilizes the channel structure and impairs channel opening without altering cilia localization and channel assembly. Our findings support the channelopathy classification of PKD2 variants associated with ADPKD, where polycystin-2 channel dysregulation in the primary cilia may contribute to cystogenesis.

Significance

How do variants that cause autosomal dominant polycystic kidney disease (ADPKD) alter the structure and function of polycystin-2 channels? This twenty year old question remains unanswered because polycystins traffic to organelle membranes, such as the primary cilia, that are challenging locations to study. Here, we focus on the molecular impact of variants found in the TOP domain of polycystin-2, a site commonly mutated in ADPKD. We report the C331S variant structure, where the TOP domain is destabilized by the localized mutation. We find that TOP domain variant channels still assemble but fail to open at normal voltages. Importantly, these variant polycystins retain their native primary cilia trafficking, suggesting their availability to channel modulators as a rationale for ADPKD treatment.

Introduction

Autosomal dominant polycystic kidney disease (ADPKD) is the most common heritable form of kidney disease¹. The disease is characterized by the development of numerous kidney cyst that often causes renal failure in midlife². Approximately ninety-five percent of cases of ADPKD are associated with variants in polycystin genes, PKD1 or PKD2, which encode for polycystin-1 and polycystin-2, respectively^{3,4}. Individuals with ADPKD often carry germline variants in one allele and the midlife disease onset is attributed to the acquisition of a second somatic mutation in the remaining allele in cystic cells^{5,6}. Polycystin-2 is a member of the transient receptor potential polycystin (TRPP) class of ion channel subunits which contain six transmembrane spanning helices⁷. Polycystin-1 is a membrane protein with eleven transmembrane spanning helices that is related to adhesion class G-protein coupled receptors and TRPP channels. Based on biochemical analysis and immunolocalization results, polycystin-1 and polycystin-2 can form a complex that traffics to the primary cilia of kidney collecting duct epithelia^{3,4,8}. Primary cilia are microtubule-based organelles that extend from the apical side of cells and amplify critical second messenger pathways⁹⁻¹¹. While two groups have independently verified that polycystin-2 is required for channel formation in the primary cilia, the contribution of polycystin-1 to the voltage-dependent, large conductance cilia current appears to be dispensable^{12,13}. However, this work does not exclude the possibility that the polycystin-1 and -2 complex is biologically relevant. Indeed, cryo-EM structures have captured polycystin-2 in its homomeric form and in complex with polycystin-1¹⁴⁻¹⁷. Recent work expressing PKD1 with PKD2 genes demonstrates that ion selectivity can be altered when polycystin-1 is incorporated, but this only occurs when polycystin-2 is trapped in an open state by mutations in pore residues¹⁸. Thus, these results do not discern if polycystin-1 is operating as a chaperone for polycystin-2 or forms a bona fide ion channel with undetermined gating properties. Since the native form of the putative heteromeric channel has escaped detection, we have focused our efforts on determining the impact of ADPKD-causing variants within the context of the homomeric polycystin-2 ion channel.

For more than 20 years, variants in polycystins have been implicated in ADPKD, yet our understanding of their impact on channel function and biosynthesis is insufficient. Cells isolated from ADPKD cysts often contain premature stop codons, or large truncations or insertions in PKD1 or PKD2^{19,20}. These drastic alterations suggest that ADPKD is caused by a loss-of-polycystin function¹⁹. This hypothesis is supported by results of rodent models of ADPKD in which haploinsufficiency and loss of heterozygosity of PKD1 or PKD2 cause kidney cyst formation in mice^{17,27-29}. Although, there are currently no mouse models harboring human disease-causing PKD2 variants, human PKD2 transgene expression can dose-dependently rescue the ADPKD phenotype in PKD2 null mice²¹. Two clinically relevant PKD2 missense variants-D511V and T721A-cause a complete loss of channel activity when measured using reconstitution assays from endoplasmic reticulum (ER) membranes²². However, using this method we learned little about how variants disrupt polycystin-2 channel mechanics and cellular localization. Paradoxically, there is evidence that transgene overexpression of PKD2 may also cause the polycystic kidney phenotype in mice²³. Furthermore, human PKD2 overexpression in mice leads to abnormalities in tubule development and eventual kidney failure²⁴. Thus, the biophysical mechanism by which ADPKD variants may cause a loss-of-function or a gain-of-function of polycystin-2 is debatable and highlights the present need to assess their impact on channel gating, structural assembly and localization to the ciliary membrane.

The published polycystin-2 cryo-EM structures provide atomic maps for locating ADPKD variants and a framework for hypothesis generation of the structural regulation of this channel¹⁴⁻¹⁷. We reported that the homotetrameric polycystin-2 TOP domains (also called the “polycystin domains”) interlock to form a unique lid-like structure that engages the channel pore from the external side¹⁷. More than 80% of the reported missense variants identified in ADPKD patients (http://pkdb.mayo.edu) can be mapped onto the TOP domain²⁵. Since the TOP domain (˜220 residues) represents a novel fold found only in polycystin proteins, it is difficult to speculate how this structure may regulate channel biophysics and biosynthesis. Based on structural observations, we propose two competing—but not mutually exclusive—mechanistic hypotheses regarding the impact of these variants. First, the TOP domain forms a molecular bridge between the voltage sensor domain (VSD) and the ion conducting pore domain (PD). Here, the TOP is well-positioned to transfer voltage-dependent conformational changes in the VSD to open the channel pore, and we hypothesize that TOP domain variants will disrupt polycystin-2 channel gating. Second, the TOP domain also forms homotypic contacts between each subunit, which likely stabilize the oligomeric channel structure. Since there is strong precedence for external domains being essential for tetrameric ion channel formation and native membrane localization, we hypothesize that TOP domain variants may disrupt the channels' structure, assembly and trafficking to the primary cilia²⁶⁻²⁸. Thus, the affect of ADPKD mutations found in the TOP domain forms the basis of the present study.

Located within the TOP domain, the finger 1 motif is the site of five, highly pathogenic, germline missense variants (K322Q/W, R325Q/P and C331S) in the human ADPKD population. In the present study, we report the cryo-EM structure of the C331S variant, which distorts the structure of the finger 1 motif and the TOP domain. Despite these changes in atomic structure, C331S and the other finger 1 variants do not alter polycystin-2 channel assembly and localization to the cilium as visualized through super resolution microscopy. However, all five variants cause a loss of ion channel function as measured by voltage clamping the primary cilium membrane. We report that analogous mutations in the related polycystin-2L1 (PKD2-L1) channels cause a loss of function, demonstrating that the conserved interactions are necessary for the function of TRPP subfamily members. Based on our previously published cryo-EM structure of polycystin-2, we know the C331 variant site participates in a disulfide bond found within finger 1 of the TOP domain¹⁷. We report that breaking the finger 1 disulfide bond, either by mutation or chemical reduction, causes disorder in the TOP domain and facilitates channel closure by shifting the voltage dependence to depolarizing potentials. This work identifies the molecular dysregulation of polycystin-2 channels that underlies forms of ADPKD caused by finger 1 motif variants in PKD2. Our results demonstrate that the TOP domain is essential for voltage-dependent gating, but that sites found within the finger 1 motif do not alter channel assembly and ciliary trafficking.

Results

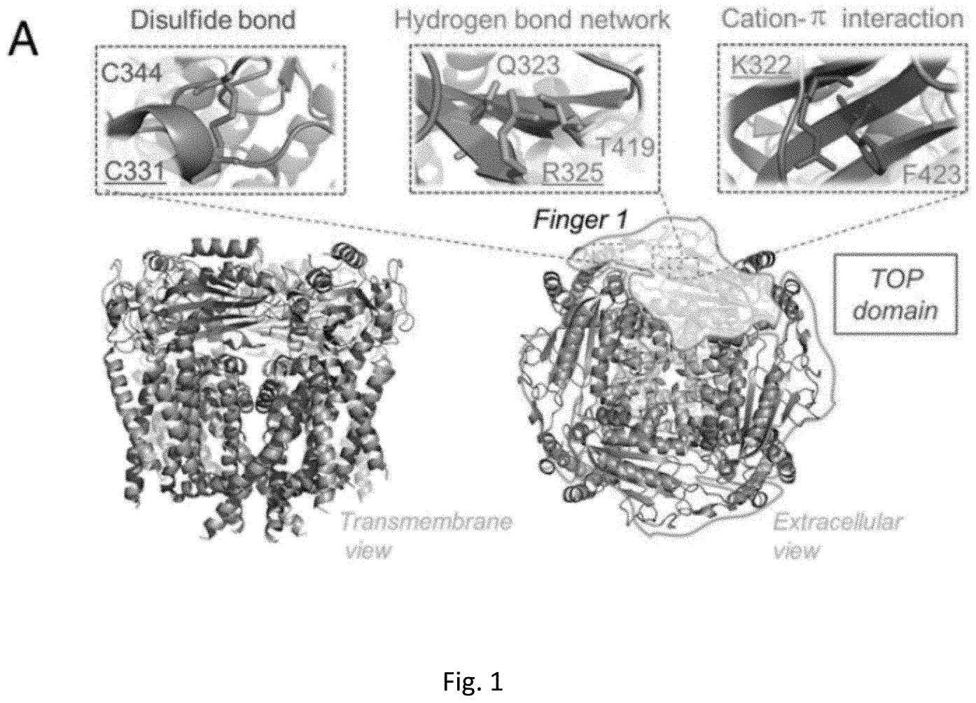

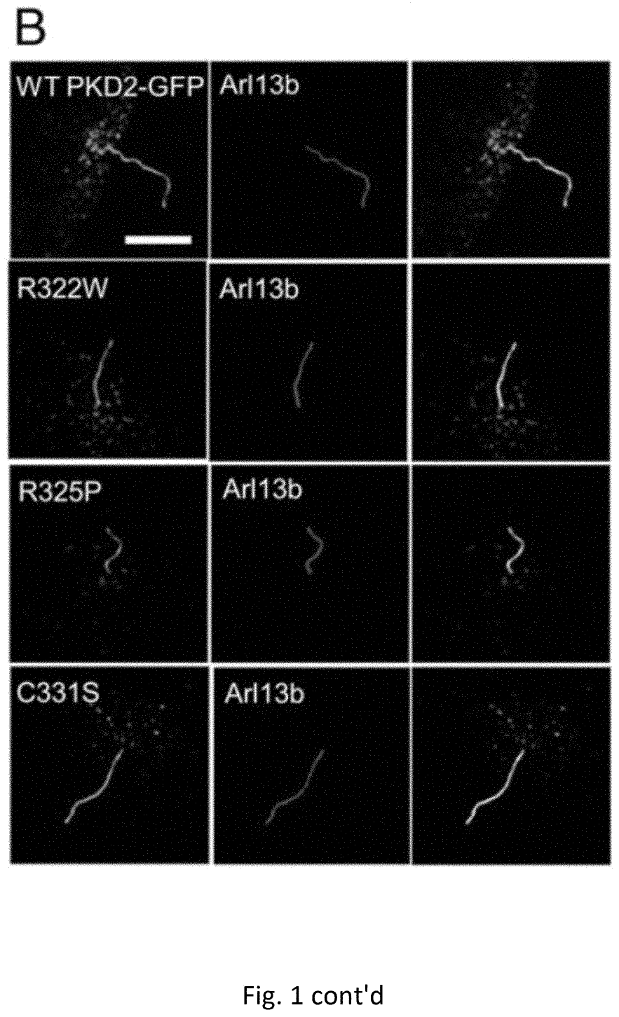

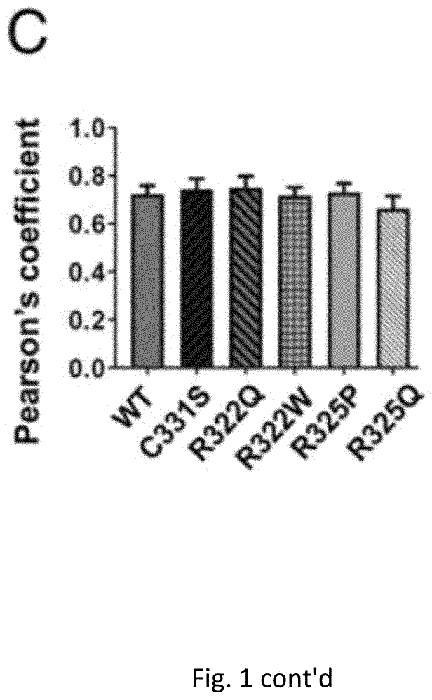

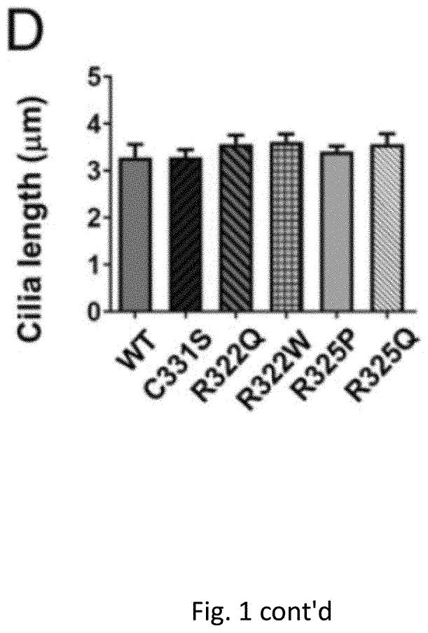

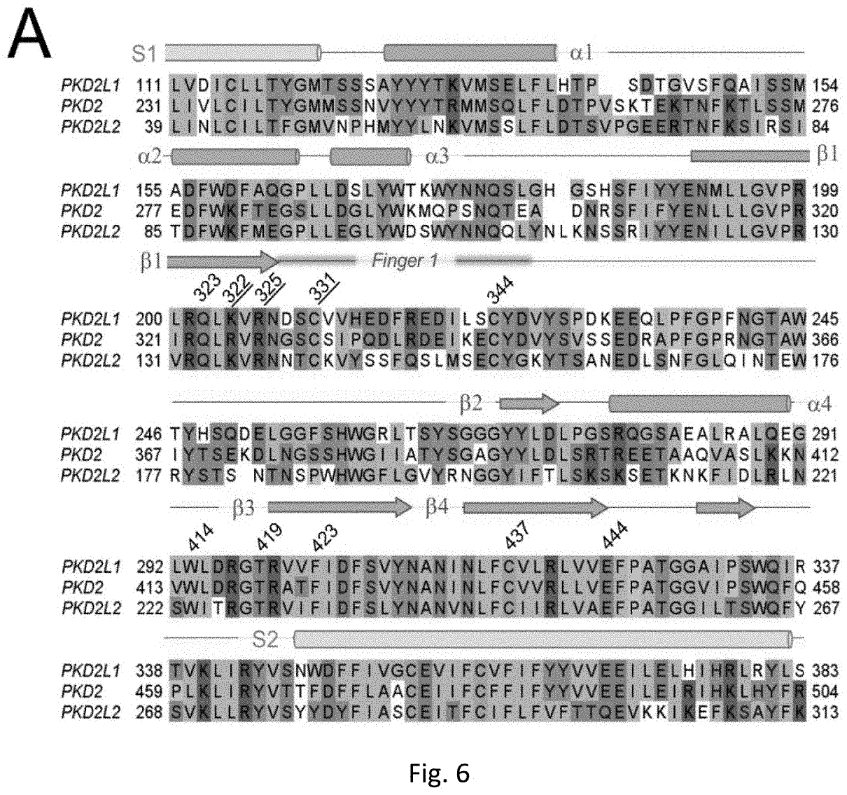

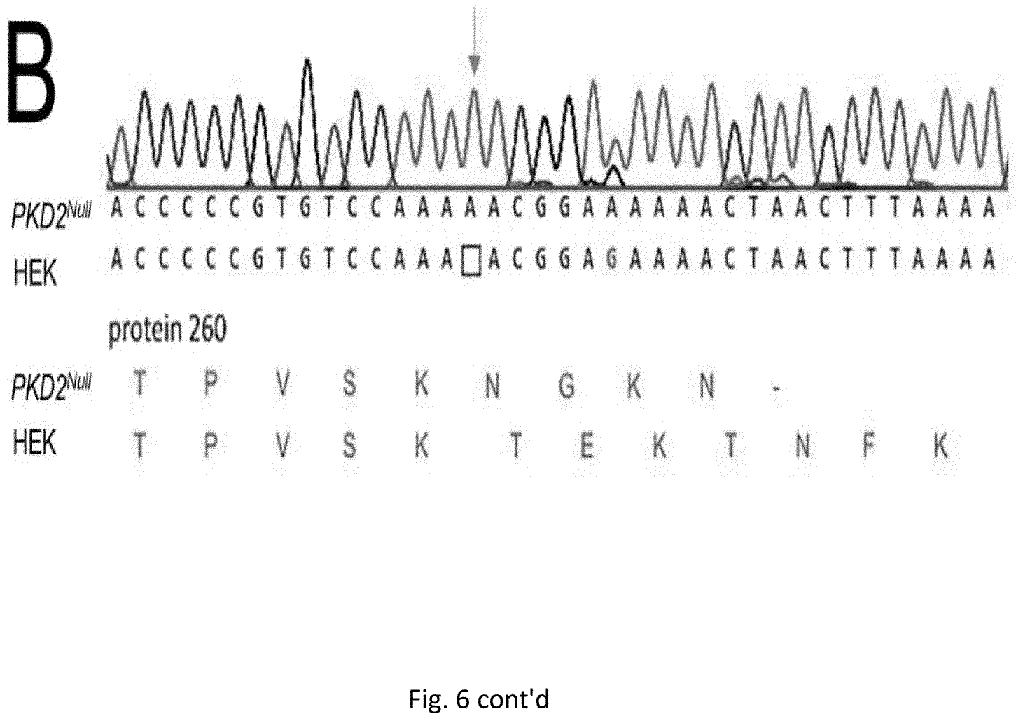

Proposed atomic interactions disrupted byADPKD-causing variants in TOP domain's finger 1 motif Previously, the polycystin-2 homotetrameric structure embedded in lipid nanodiscs was solved at 3 Å resolution using single particle cryo-electron microscopy¹⁷. Here, we described the TOP domain (also called the ‘polycystin domain’ in the initial publication) which forms an extracellular, lid-like structure in which >80% ADPKD-causing PKD2 missense variants are found. The TOP domain (residues 242-468 between the Si and S2 helices) forms a novel protein fold comprised of three a helices, a five stranded anti-parallel 3 sheet which together form two inter-subunit interaction motifs called finger 1 and finger 2 (FIG. 1A, FIG. 6A). Finger 1 is formed by a hairpin turn and interfaces extensively with the β-turn protruding from the adjacent TOP domain. Within finger 1, a disulfide bond is found between the sulfur atoms of C331 and C344 which we hypothesize provides structural integrity to the hairpin and is necessary for its interaction with the adjacent TOP domain (FIG. 1A, FIG. 6A)¹⁷. C331S is an ADPKD-causing variant whose hydroxyl side chain is unable to form the disulfide bond with C344. Finger 1 extends from the palm of the TOP domain which is comprised of the central anti-parallel beta-sheet and is stabilized by a hydrogen bond network involving Q323-T419-R325 and a cation-π interaction formed between R322-F423 (FIG. 1A). Importantly, multiple ADPKD causing missense mutations are found at positions R322 (R322Q and R322W) and R325 (R325Q and R325P)²⁵. We hypothesize that these pathogenic mutations would destabilize the intersubunit interactions required for channel assembly and may disrupt polycystin-2's native cilia localization. On the other hand, these variants may destabilize the central anti-parallel beta-sheet of the TOP domain, which oligomerize and engage the pore on the extracellular side of the channel, altering the channel's ability to gate (open and close).

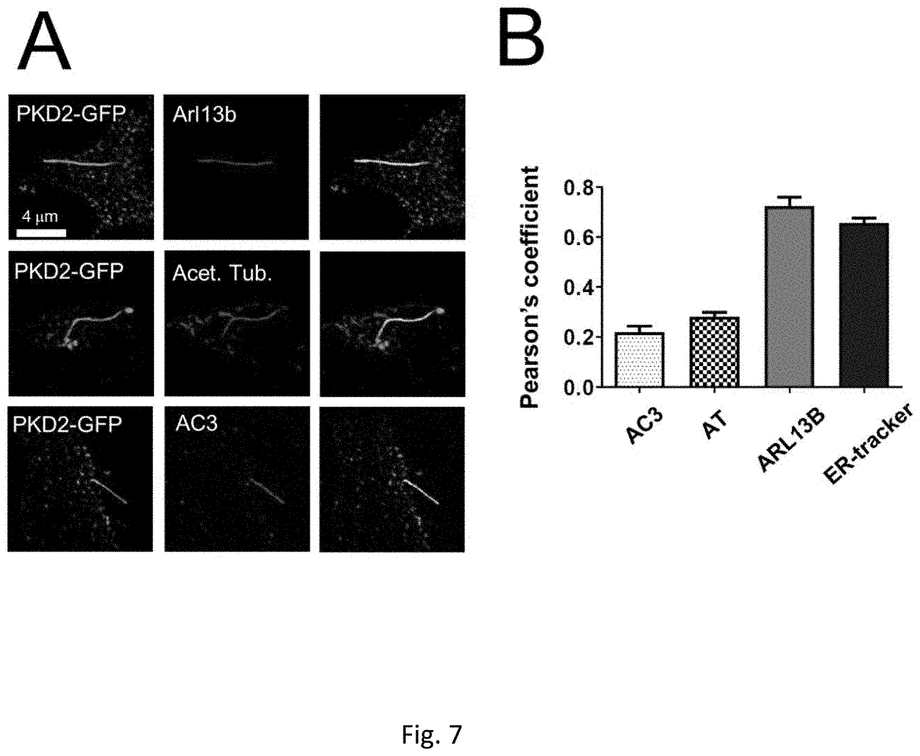

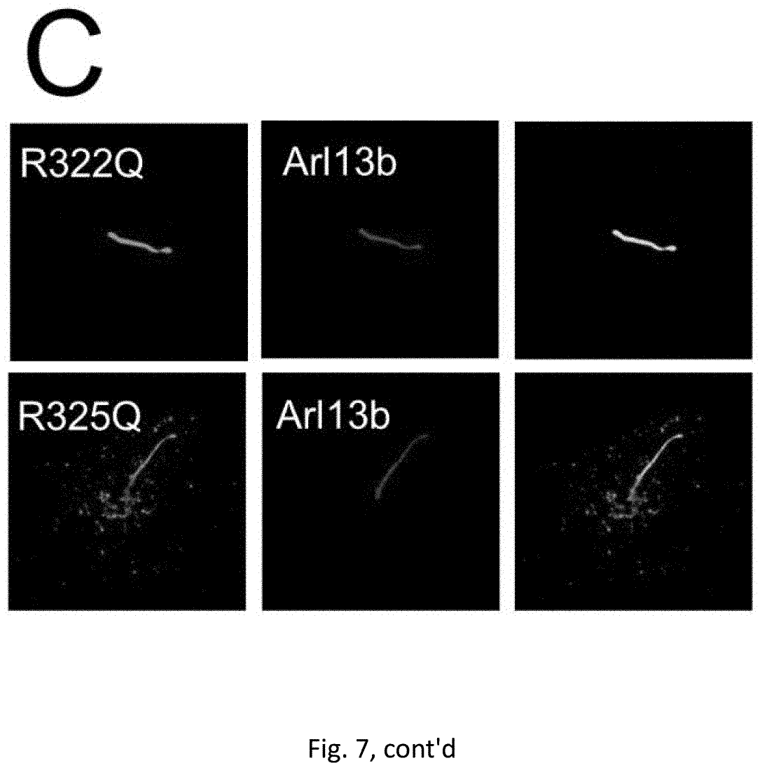

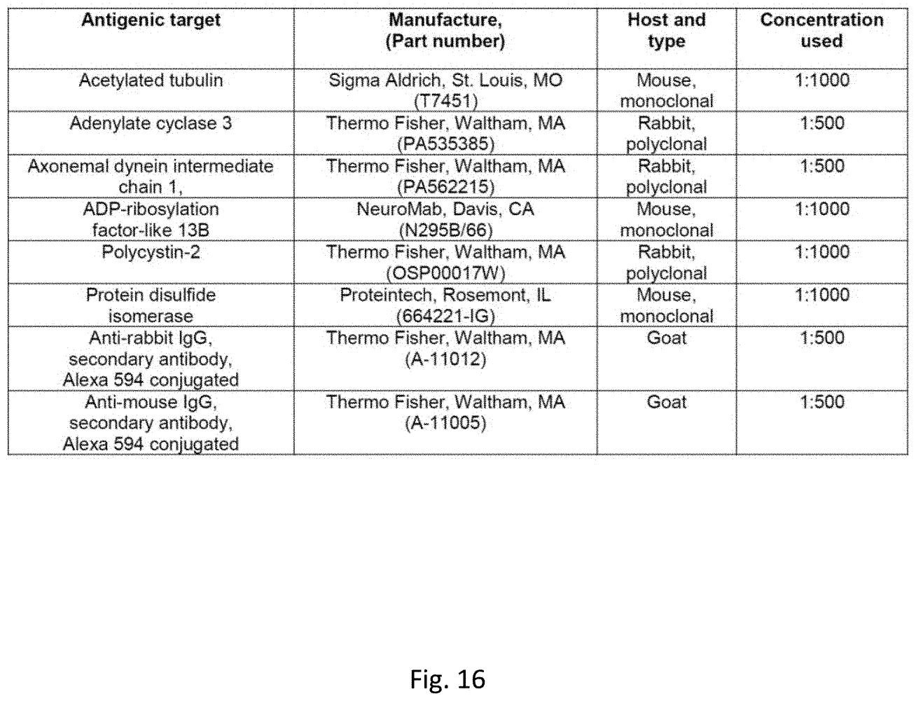

TOP domain finger 1 variants do not alter ciliary trafficking. Contrary to initial reports, expression or co-expression of PKD1 and PKD2 does not produce ionic currents on the plasma membrane of native collecting duct cells or in heterologous systems. As characterized previously, we established a method to test heterologous C-terminally GFP tagged PKD2 (PKD2-GFP) channel trafficking and function to the cilia membrane of HEK cells¹³. We have improved this system by genetically ablated the endogenous PKD2 expression in our HEK cell line (PKD2^{Null}) using the CRISPR/Cas9 method (clustered regularly interspaced short palindromic repeats) so that the impact of variants can be assessed without the contribution of endogenous alleles (FIG. 6B). Then we stably introduced PKD2-GFP to observe its subcellular distribution using structured illumination microscopy (SIM; FIG. 7A). SIM axial resolution is ˜150 nm, which is twice that of confocal microscopy and enables a more detailed view of the primary cilium, which is less than 400 nm in diameter²⁹. Here, PKD2-GFP trafficked to the PKD2^{Null} cell primary cilia, as assessed by GFP co-localization with antibodies raised against epitopes ADP ribosylation factor like GTPase 13B (ARL13B), adenylate cyclase 3 (AC3), and acetylated tubulin (AT) (FIG. 16). Analyses of these images indicate that PKD2-GFP signal had the highest Pearson's coefficient with ARL13B, thus we used this marker as a benchmark to assess the effects of ADPKD variant effects on polycystin-cilia localization (FIG. 7B). We generated six stable cell lines expressing the uncharacterized finger 1 variants (R322Q, R322W, R325Q, R325P and C331S). However, none of the variants impacted primary cilia trafficking or length when compared to the WT channel (FIG. 1C, D), demonstrating that the TOP domain mutations do not alter cilia maintenance or the channel's native localization in the cell.

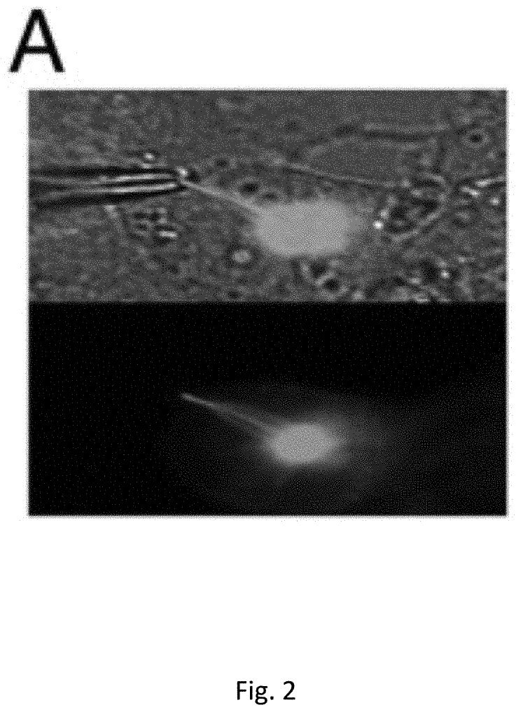

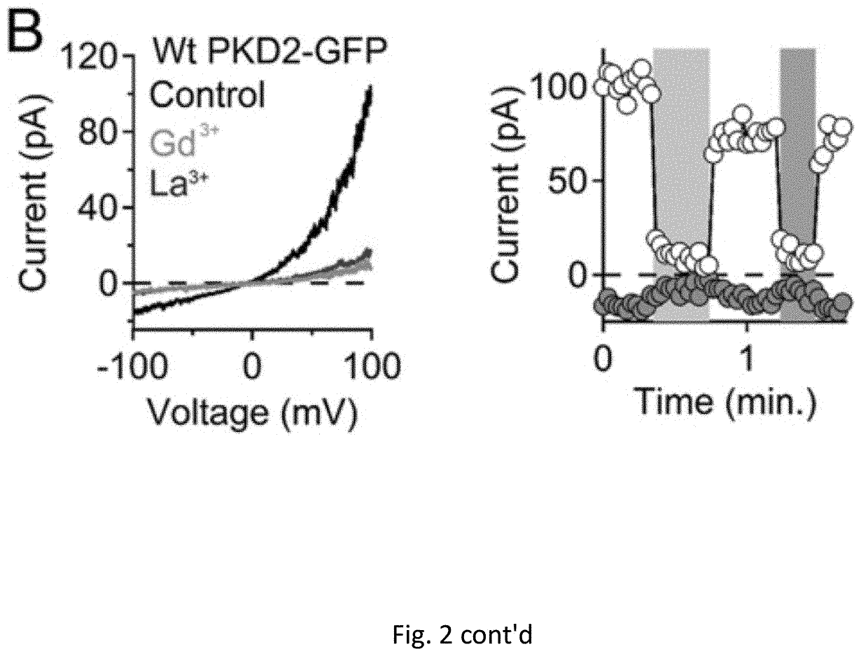

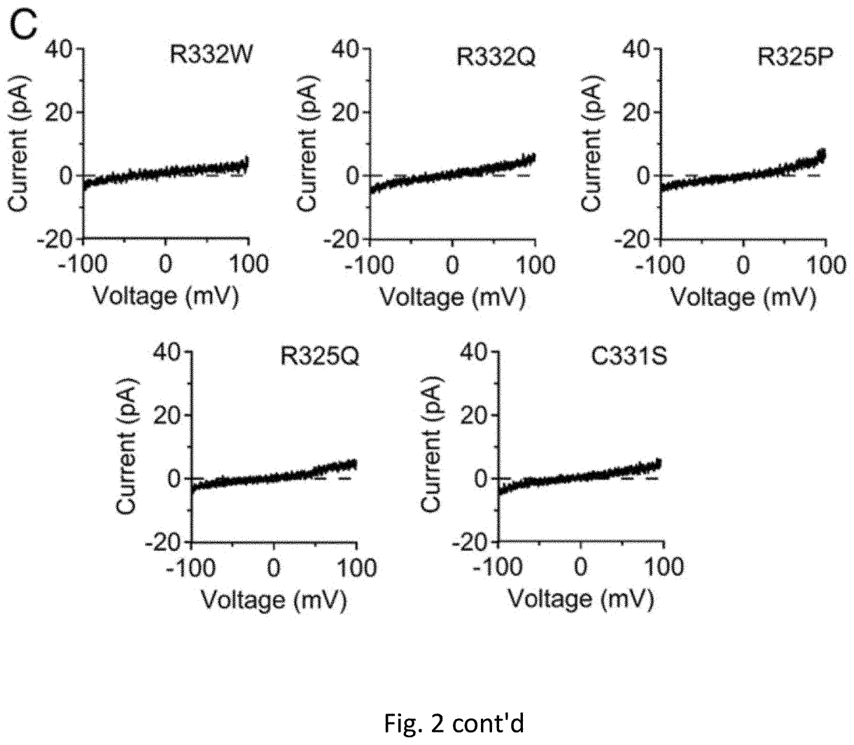

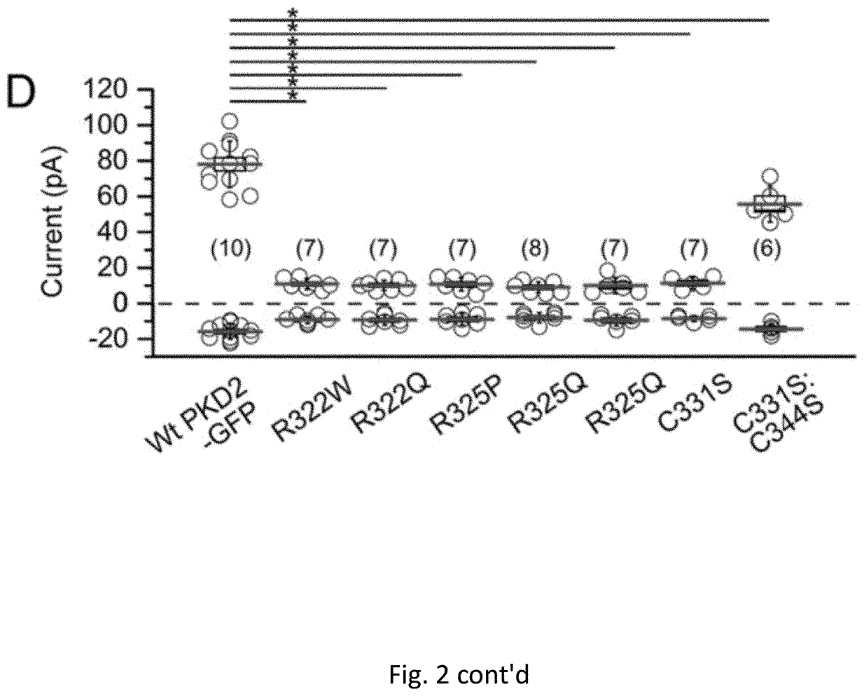

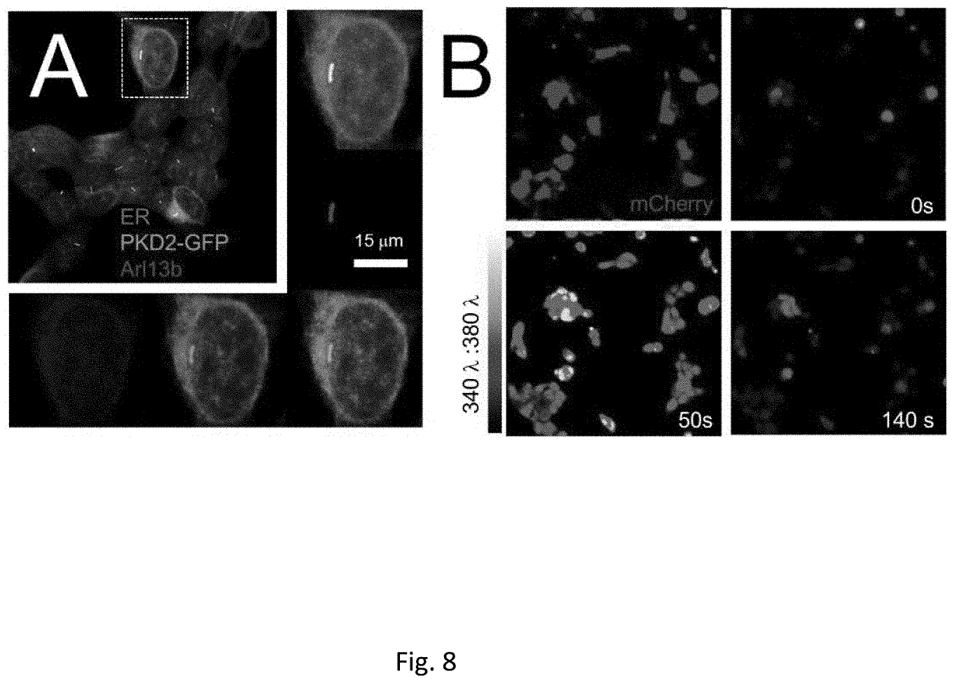

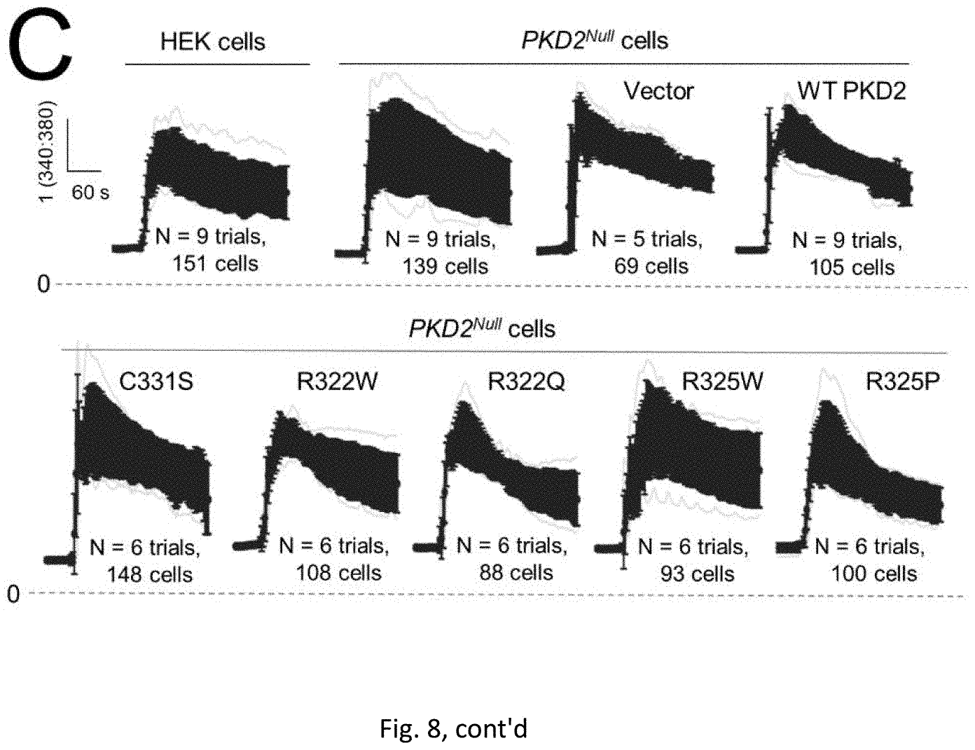

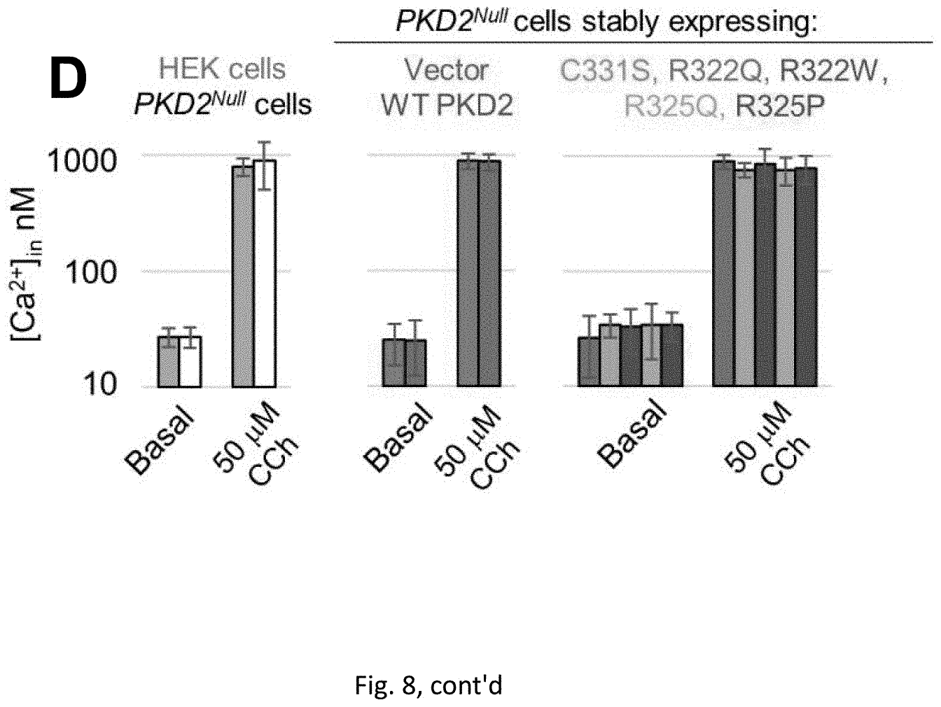

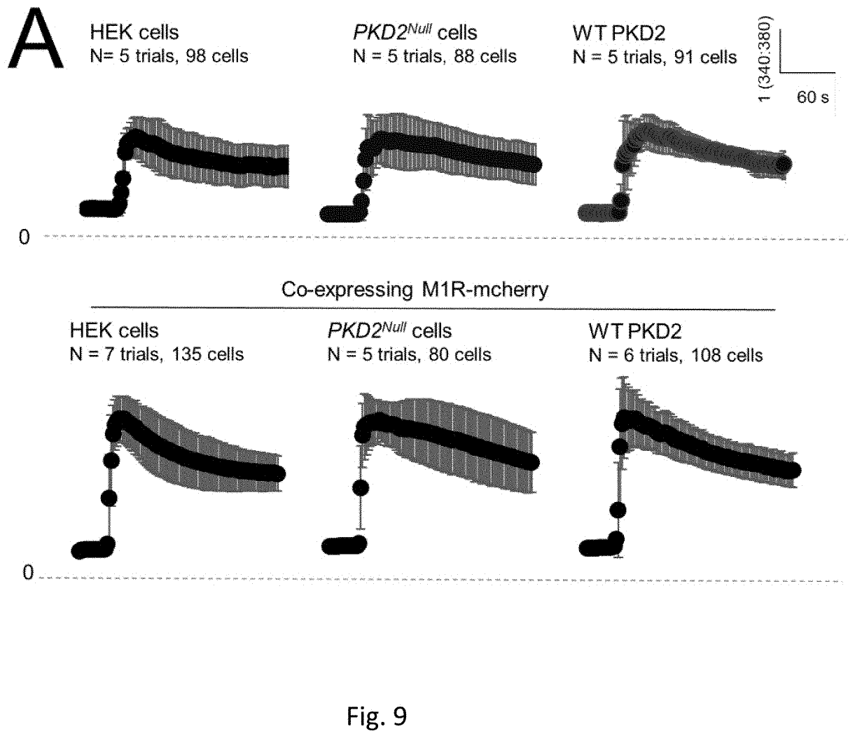

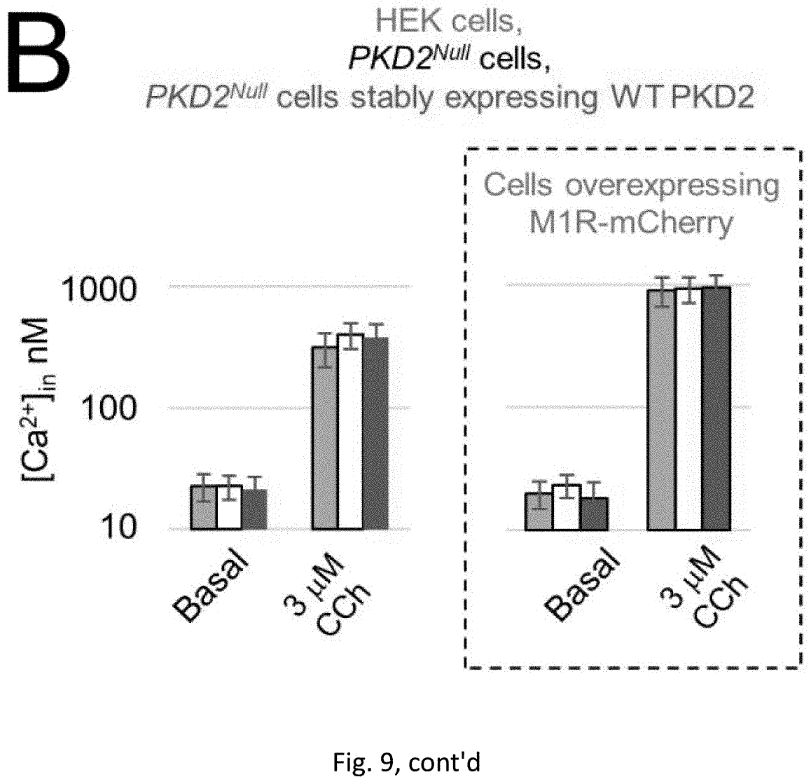

Finger 1 variants cause a loss of channel function in the primary cilia. Next, we sought to determine if the variants alter polycystin-2 channel activity in the primary cilia and ER membranes. Since the WT channel and the finger 1 variants did not alter PKD2-GFP cilia localization, we could thus visualize the HEK cilia to establish electrophysiology recordings of the channels directly from the cilia membrane, as previously reported (FIG. 2A)¹³. Here, the heterologous WT channel PKD2-GFP cilia current is outward rectifying when activated by voltage ramps and is blocked by external trivalent ions gadolinium and lanthanum-matching the pharmacology observed in native polycystin-2 currents measured from pIMCD cilia (FIG. 2B)¹³. However, the recordings from the cilia of all five variant cell lines resulted in an apparent loss of polycystin-2 current (FIG. 2C, D). Evidently, finger 1 variants disrupt channel gating in the primary cilium without affecting its ciliary trafficking. Since the primary cilia is absorbed or shed during active cell mitosis, PKD2 expression is primarily confined to the endoplasmic reticulum during this non-ciliated stage of cell division^{30,31}. Indeed, the ER population of polycystin-2 channels localized with the ER membrane when immunolabeled with ER-tracker (FIG. 8A). Previous work using cytoplasmic Ca²⁺ fluorescence dyes supports the hypothesis that polycystin-2 can function as a Ca²⁺ store-release channel in the ER, potentiating the response of the inositol trisphosphate receptor (InsP3R) in kidney collecting duct cells¹⁹. To determine if our variants alter the activity in the ER-localized polycystin-2 population, we activated the endogenous muscarinic acetylcholine receptor M1 (MIR) and assayed (50 μM) carbachol stimulation of the InsP3R-mediated Ca²⁺ store release using the cytoplasmic ratiometric calcium indicator, Fura-2 (FIG. 8B). However, we did not observe any difference in carbachol-mediated Ca²⁺ release from intracellular stores of PKD2^{Null} cells or when PKD2-GFP was stably overexpressed (FIG. 8C, D). In addition, there was no difference in the Ca²⁺ store release kinetics and maximal response between cells stably expressing WT and the TOP domain variant channels (FIG. 8C, D). We also tested calcium responses elicited from a lower dose of carbachol (3 μM) in cells expressing endogenous MIR and when the receptor was overexpressed (FIG. 9A, B). Again, no difference was observed from parental HEK cells, PKD2^{Null} cells and PKD2^{Null} cells over-expressing PKD2-GFP. Since our results using this method did not reproduce polycystin-mediated potentiation of Ca²⁺ store release reported in native cells (see discussion section), we directed our remaining experiments towards understanding how these variants impact polycystin-2 activity from the primary cilia membranes.

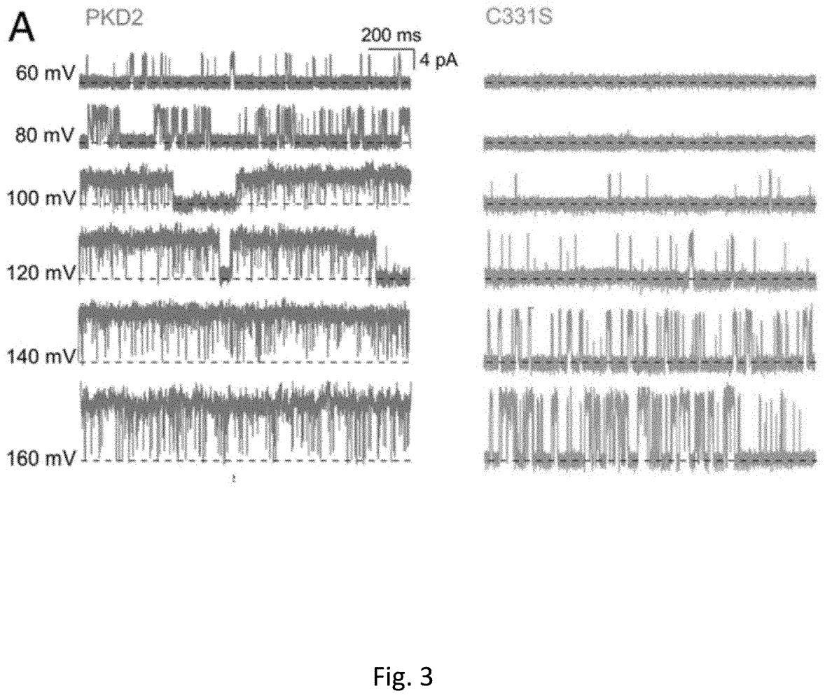

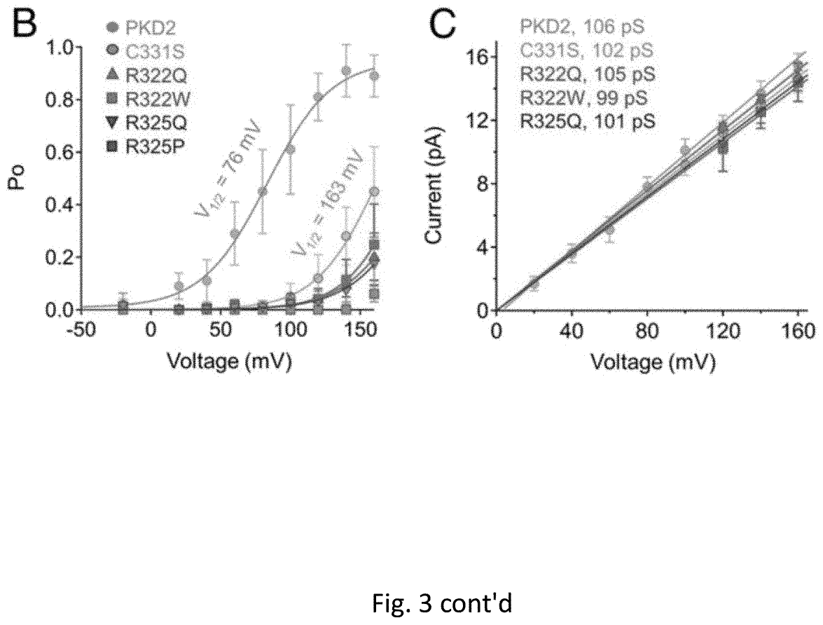

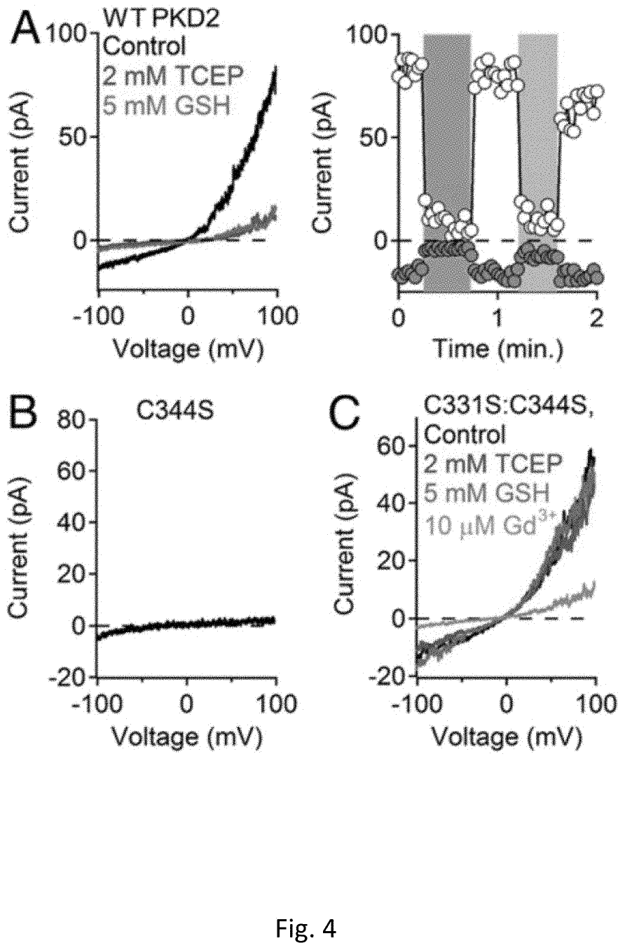

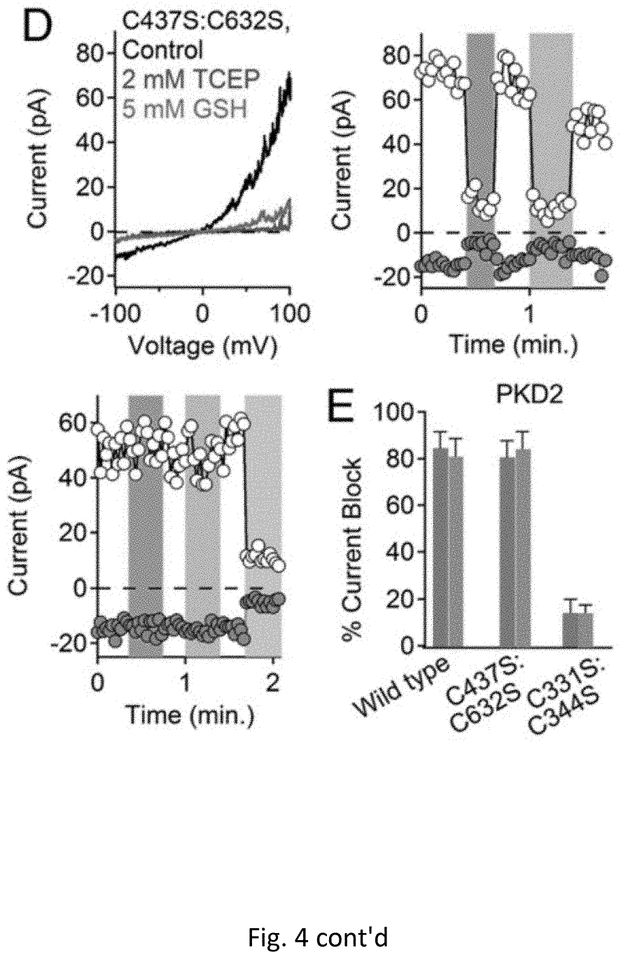

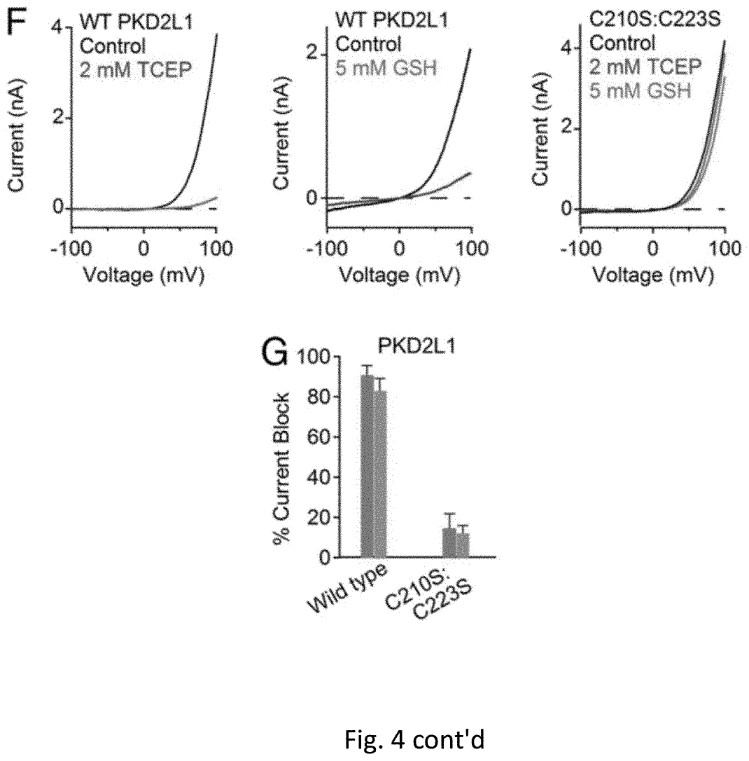

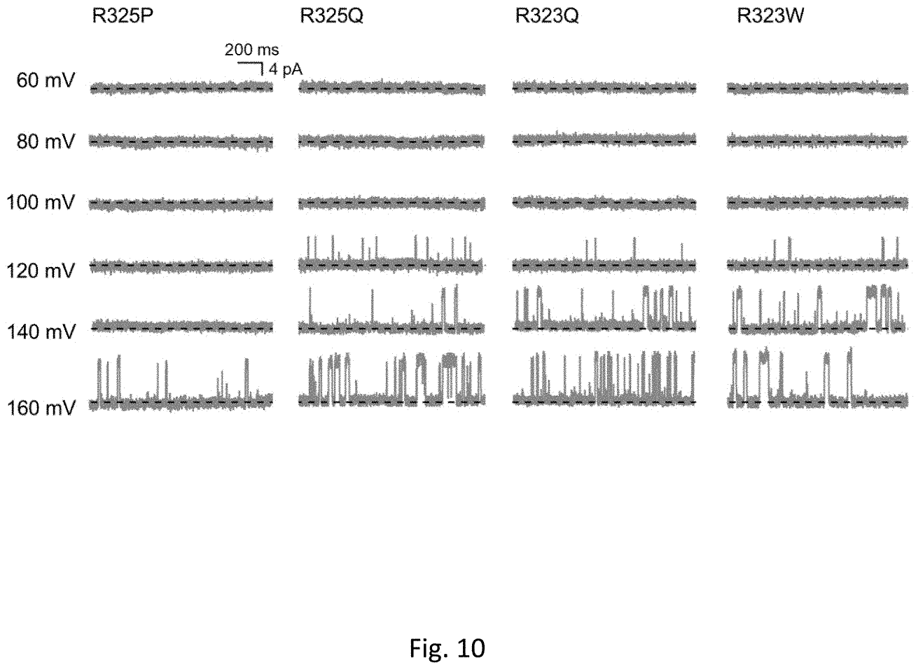

Finger 1 variants impair voltage-dependent gating. To examine the biophysical mechanisms which cause loss-of-function observed in the five finger 1 variants, we recorded single channel currents in the ‘on-cilia’ patch configuration at potentials up to 160 mV (FIG. 3A, B, and FIG. 10). Here we observed that the voltage dependence of channel opening (V I₂) for all of the variant channels were positively shifted by 87 mV or more compared to WT channels. Results from the single channel recordings explains the lack of whole cilia current measured by depolarizing voltages tested to 100 mV, because the threshold to activate the finger 1 variant channels is more depolarized. We did not observe a significant difference in the single channel conductance between the WT and the variant channels, indicating that the pore properties were unaltered (FIG. 3C). Next, we tested the effects of chemically breaking the finger 1 C331-C344 disulfide bond in real-time while recording polycystin-2 currents in the ‘whole cilia’ configuration. We applied external disulfide reducing agents tris-2-carboxyethyl phosphine (TCEP) or reduced glutathione (GSH), and observed near instantaneous reduction in the wild type polycystin-2 current that was readily reversible (FIG. 4A). TCEP and GSH were selected because they are membrane impermeable, having octanol:water partition coefficients of −4.9 and −4.7 respectively and could only reduce the extracellular accessible disulfide bonds. Since the ADPKD-causing variant C331S causes a loss-of channel function, we proposed that mutating its disulfide interacting partner, C344, would have a similar effect. Serine was substituted for C344 because its side-chain hydroxyl has a similar volume as cysteine sulfhydryl atoms, thus this substitution would likely be tolerated at this position but incapable of forming a disulfide bond. As expected, stably expressing C344S failed to produce cilia currents (FIG. 4B). While these results implicate the sites as important for polycytin-2 functions, they do not demonstrate that their interaction is required for their function. Thus, we introduced an artificial hydrogen bond interaction at these positions to see if it would rescue the cilia current. We replaced both positions C331 and C344 with serine within the same channel (C331S:C334S). Now, the native interacting sulfhydryl side chains are replaced with hydroxyl residues that can form a hydrogen bond that is not dependent on redox conditions. As expected, C331S:C334S channel generates a functional cilia current which is resistant to antagonism by external reducing agents, while retaining its sensitivity to pore block by gadolinium (FIG. 4C, E). The results from this rescue experiment demonstrates that an interaction at this site, disulfide or a hydrogen bond, is necessary for polycystin gating. However, the effect of the antagonism of polycystin-2 by reducing agents could be attributed to hydrolysis to other cysteine pairs within the channel. While there are two other extracellular located cysteine residues found in polycystin-2 (C437 and C632), neither of these resides participate in disulfide bonds. C437 resides in fourth strand of the antiparallel 3 sheet of the TOP domain and C632 is found in the lipid facing side of the first pore helix (P1, FIG. 6A). We substituted both positions with serine resides by generating C437S:C632S double mutant channel and found that the double mutant channel remained functional in the cilium and retained its sensitivity to GSH and TCEP-thus eliminating the possibility that these are responsible for the observed effect (FIG. 4D, E). Taken together, we conclude that breaking C331-C344 interaction either by chemical reduction or sulfhydryl substitution results in a loss of polycystin-2 channel function by shifting the channels voltage dependence to depolarizing potentials.

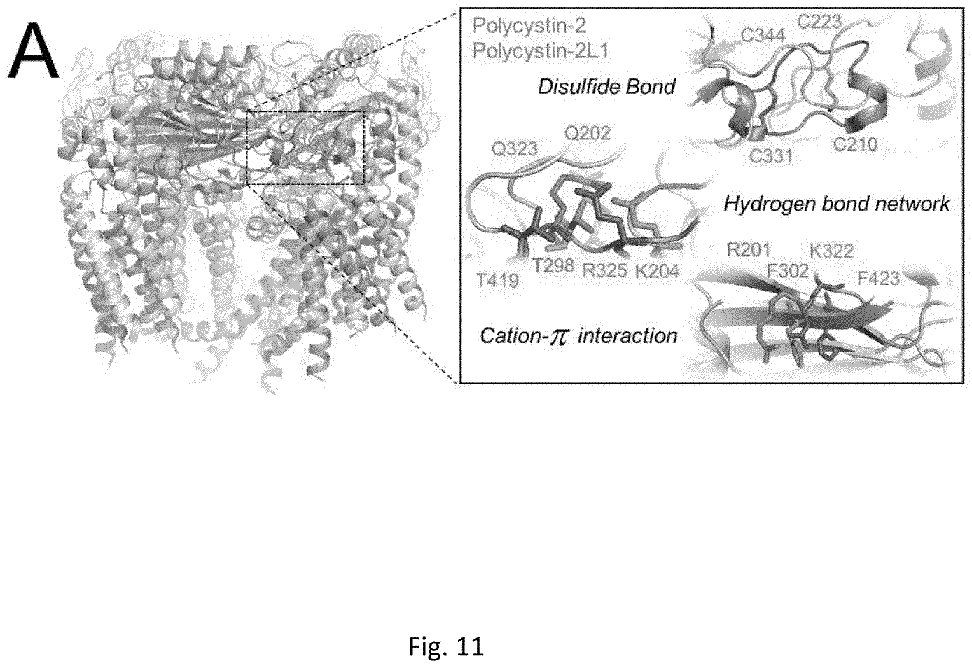

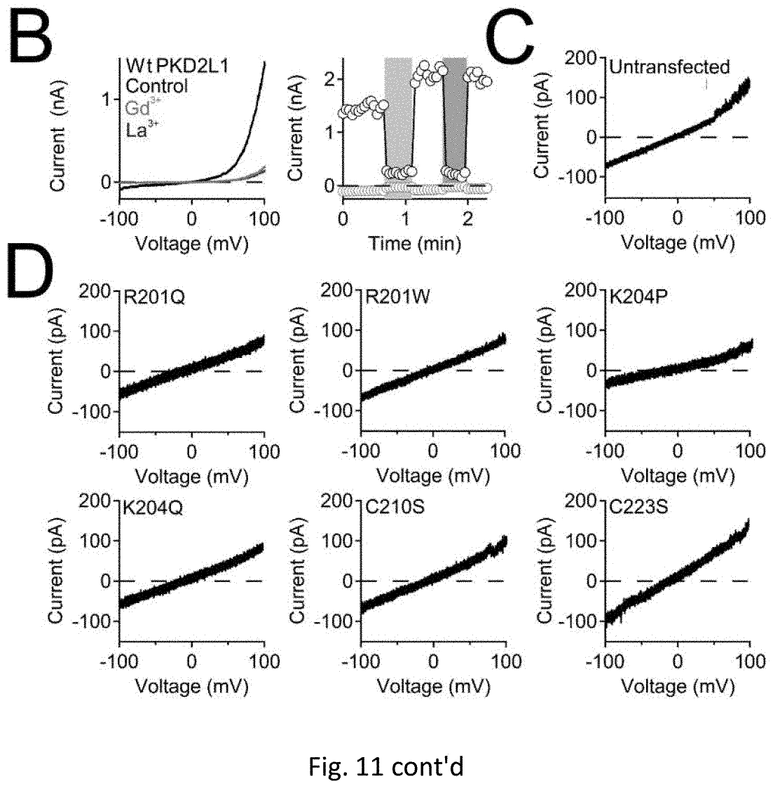

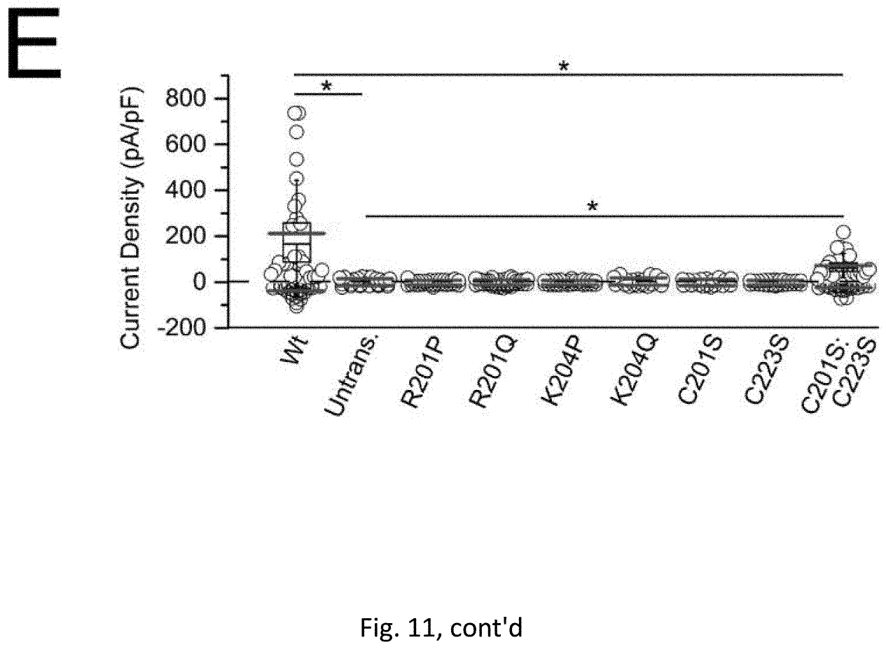

Conserved TOP domain molecular interactions are required for polycystin-2L1 gating. Heterologous expression ofPKD2L1 encodes for functional polycystin-2L1 ion channels in the plasma membrane, whereas PKD2 does not³². Previously, we reported expression of PKD2L1 is required for the ciliary conductance of non-renal cells (e.g. retina pigmented epithelia cells), whereas PKD2 is required for cilia current recorded from kidney tubule epithelial cells. Thus, while both polycystin-2 and polycystin-2L1 can form ion channels in primary cilia, their cilia expression and localization are dependent on tissue type. Based on sequence alignments and structural analysis of the cryo-EM structures of poly cystin-2 and poly cy stin-2L1, the finger 1 variant sites and their contributing interactions are conserved in both channels (FIGS. 6A, 10)^{33,34}. Therefore, we tested polycystin-2L1 channel mutations R201Q, R201W, K204Q, K204P and C210S—which are equivalent to the R322Q, R322W, R325Q, R325P and C331S variants in the polcystin-2 channel, respectively. Transient overexpression ofPKD2L1 (poly cystin-2L1) generates an outwardly rectifying current in the plasma membrane that is sensitive to trivalent block (FIG. 11B). However, introducing any one of the finger 1 variants resulted in a complete loss of the current (FIG. 11C-E). These findings suggest these TOP domain finger 1 interactions are required for both polycystin channel types. The finger 1 disulfide of polycystin-2L1 is formed between residues C210 and C223, and substituting either cysteine results in a loss of channel function-echoing results from the polycystin-2 channel (FIG. 11C). As done for the ciliary polycystin-2 channels, we chemically reduced the polycystin-2L1 finger 1 disulfide bond by applying the extracellular TCEP or GSH and observed a near complete loss of current, which was readily reversible (FIG. 4F). Replacing the finger 1 disulfide interaction with a hydrogen bonding pair, C210S:C223S, partially restored its conductance and rendered the channel insensitive to GSH (FIG. 4F, G, FIG. 11E). The results from the polycystin-2L1 channels recapitulate our polycystin-2 findings, where an interaction at these sites within Finger 1 is required to open the channel.

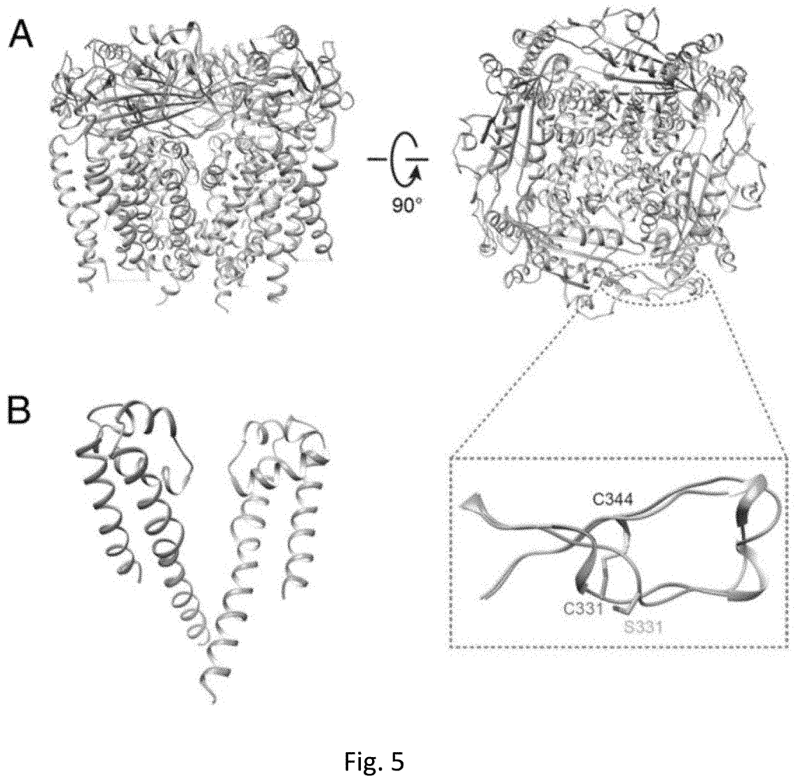

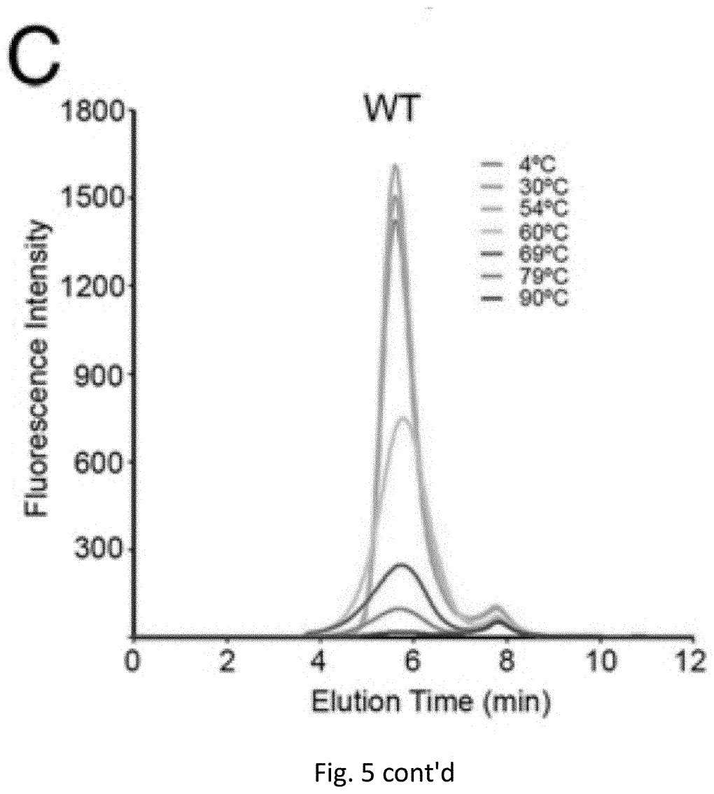

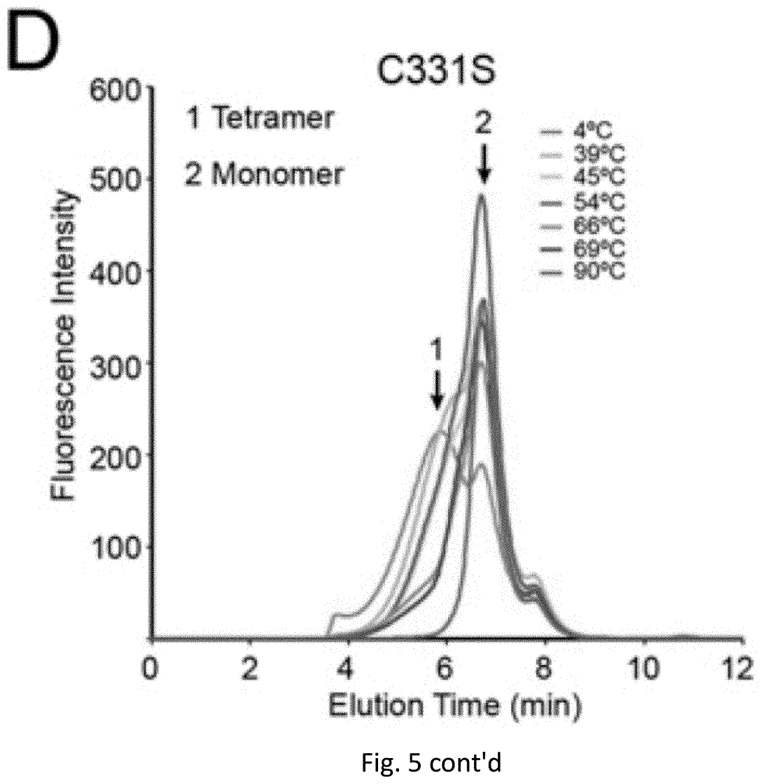

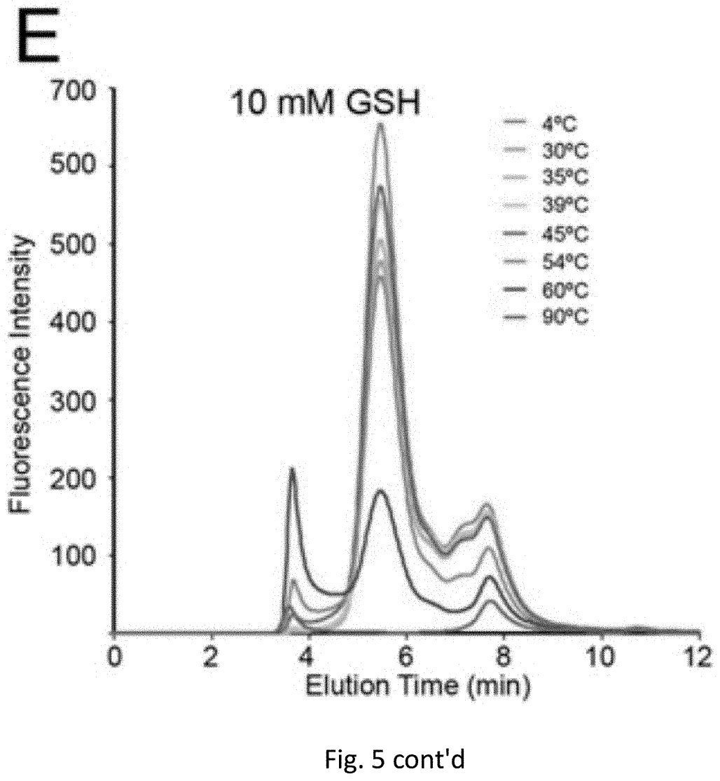

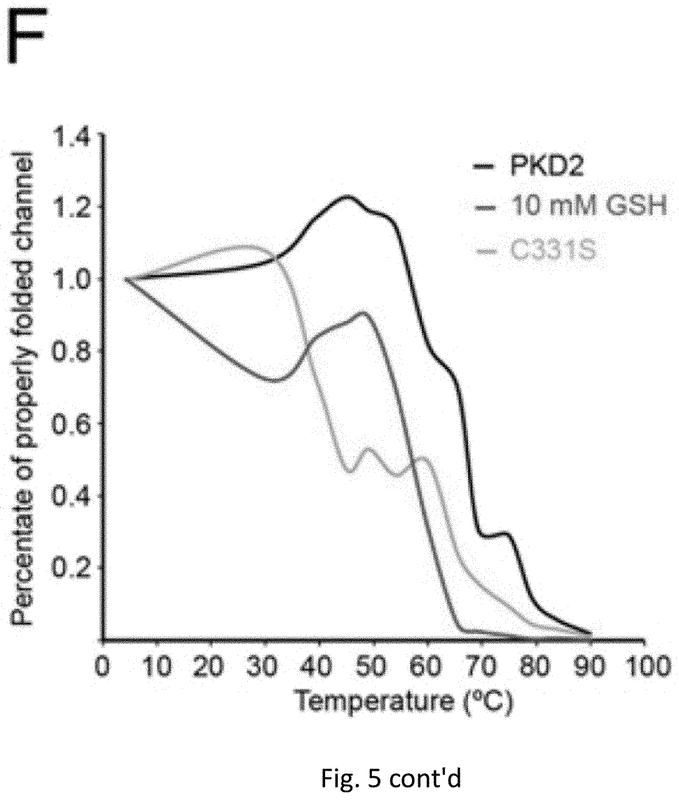

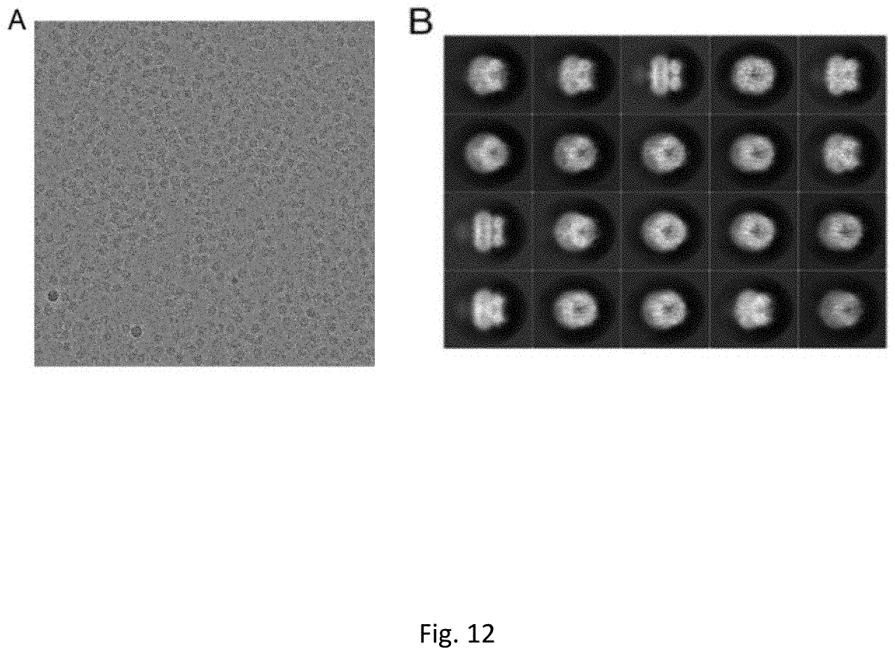

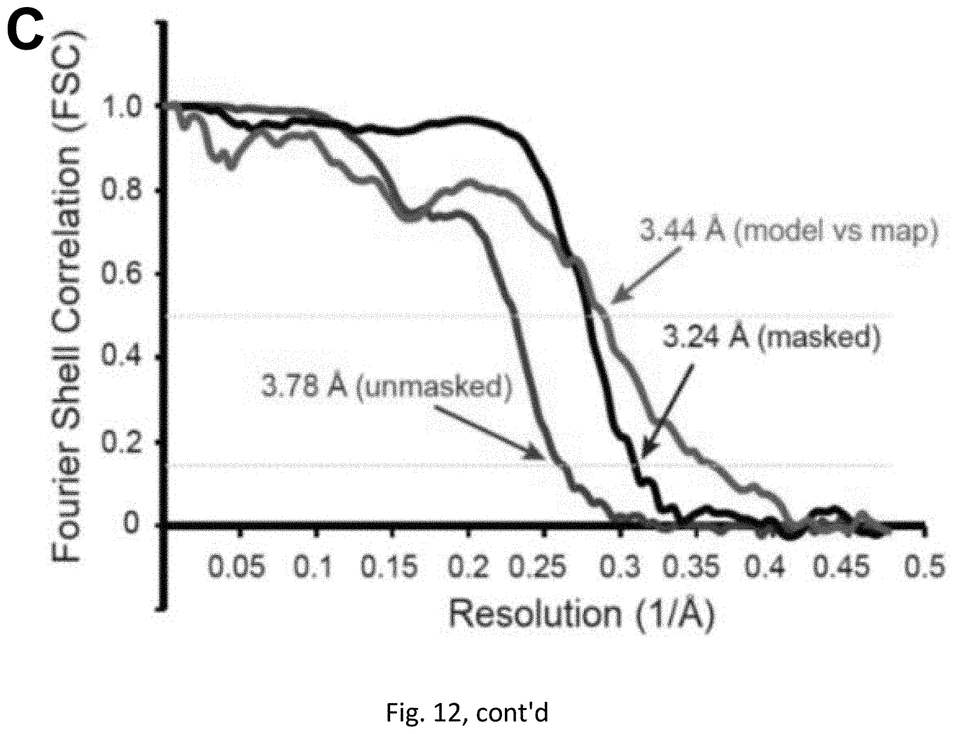

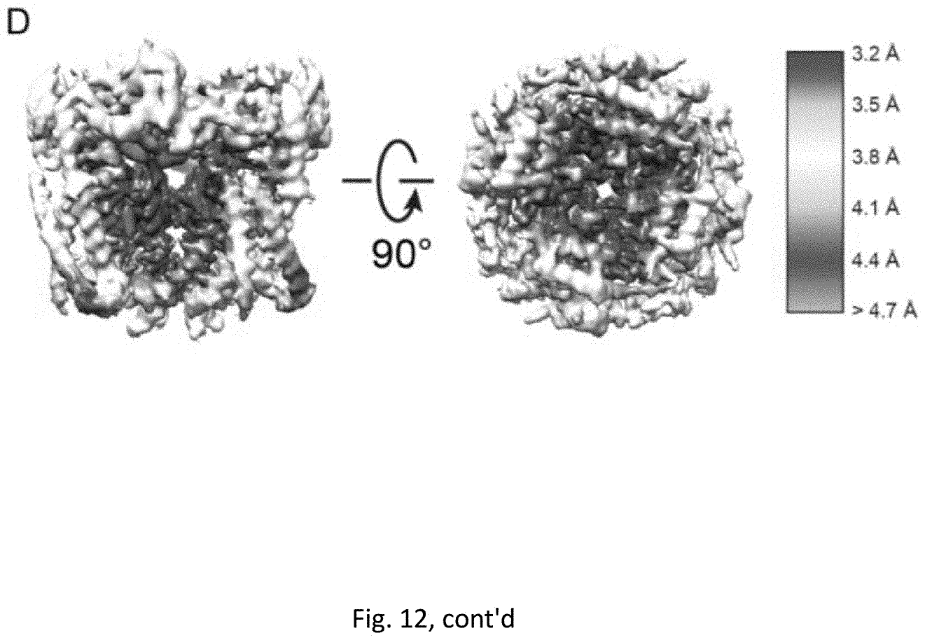

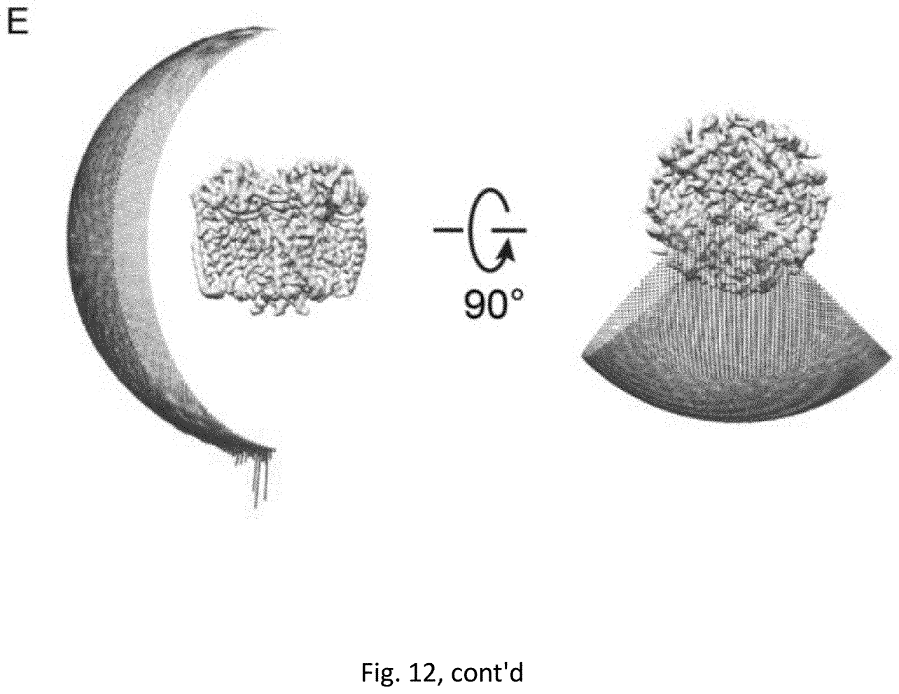

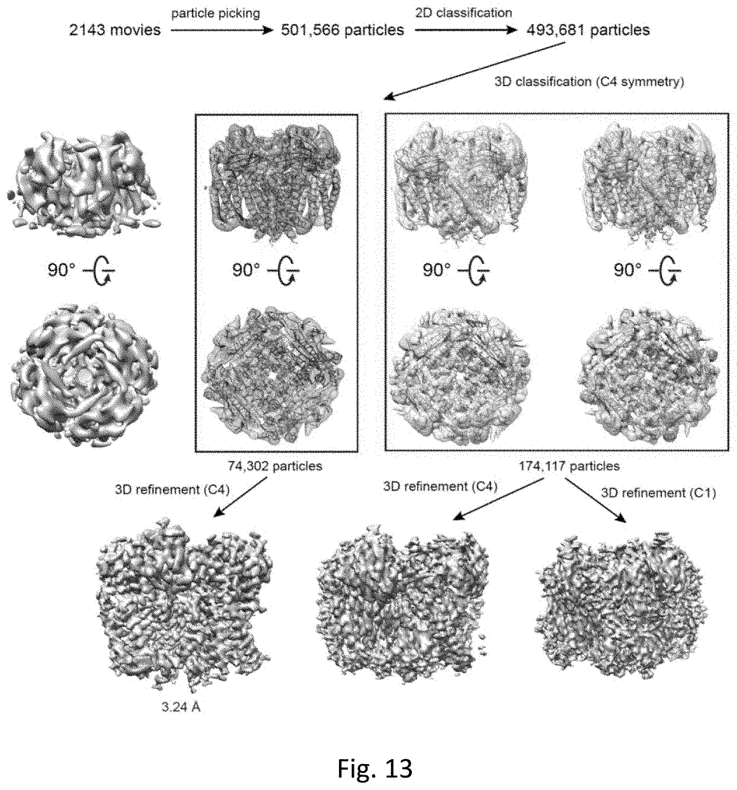

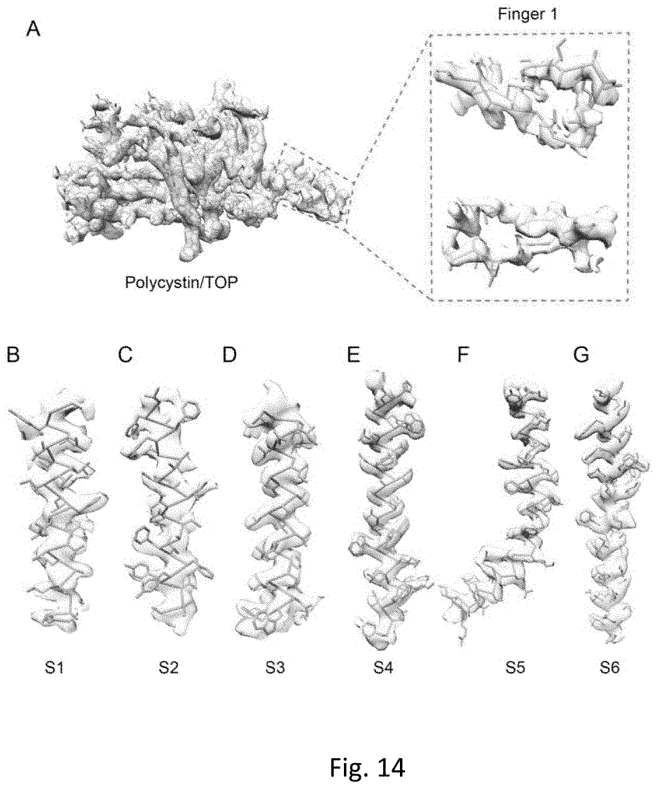

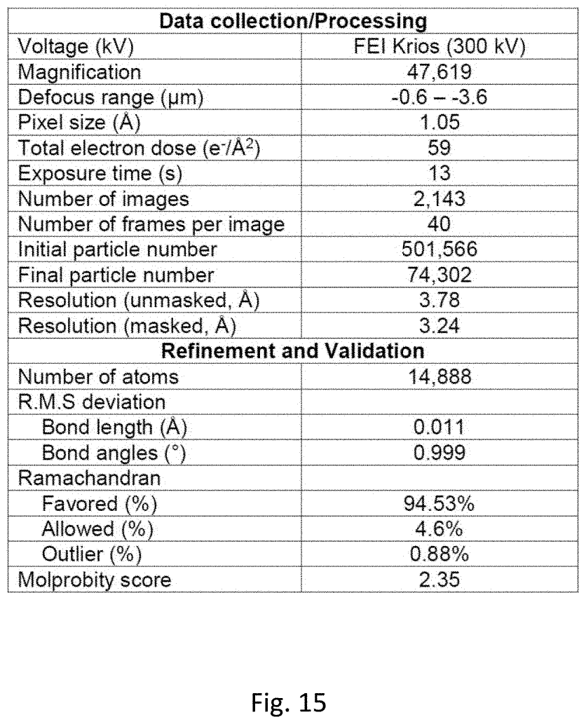

Destabilization of the TOP domain in the cryo-EM C331S variant channel structures. To determine if disrupting TOP domain interactions alters channel assembly and stability, we expressed and purified the C331S variant of polycystin-2 (residues 53-792) protein in amphipols for structural determination using single particle cryo-EM. Two-dimension (2D) class averages showed a broad distribution of views in which the distinct channel features of the tetrameric architecture are clearly discernible (FIG. 12). Thus, the C331S mutation did not substantially impair the channel's quaternary structure. Nevertheless, further in silico 3D classification revealed two populations of particles (FIG. 13). The first structure class is refined to 3.24 Å and essentially superimposes with the wild-type channel, except lacking the disulfide bond. Surprisingly, breaking the disulfide bond only slightly altered the finger 1 hairpin (FIG. 5A, B, FIG. 14A, FIG. 15). The second class retained the tetrameric architecture as well, but the TOP domain was visibly disordered (FIG. 13). It is important to note that we have not observed this disorder in the WT or the previously reported F604P structures, even though the data processing procedures were the same^{17,35}. We also considered that the C331S variant may break 4-fold symmetry, but refinement of this second class without imposing any constraints still yielded a map with the same C4 symmetry. Using size exclusion chromatography, we observed that polycystin-2 is stable at temperatures beyond 50° C. (FIG. 5C, F) in DDM detergent. Since chemical reduction of the polycystin-2 inhibited the ciliary current, we added GSH to our purification conditions and observed channel protein denaturing at lower temperatures, suggesting that reduction of the sole disulfide bond between C331S-C344 disrupts the channel stability (FIG. 5E, F). Similarly, approximately half the C331S variant protein denatured when the sample was heated to 50° C., generating more monomeric subunits in the process (FIG. 5D, F). Our data demonstrate that the C331S variant causes subtle differences within the finger 1 structural fold, but leads to destabilization of the overall TOP domain and channel function. We did not observe appreciable alterations to the positions of the transmembrane helices between the variant and WT structures (FIG. 14), as both channels appear to be captured in a closed state. These observations are not surprising given that opening the WT and C331S channels require a positive membrane potential, which is missing from the cryo-EM conditions.

Discussion

ADPKD as a channelopathy. There are more than 400 genes which encode ion channel subunits that control the flux of ions across cell membranes. Ion channels are involved in many physiological processes, including neurotransmission, muscle contraction, secretion, immune response, cell proliferation, and differentiation³⁶. Variants in genes that encode ion channels or their interacting protein subunits, are responsible for many rare and common conditions that impact organ function³⁷. Collectively, these genetic diseases are called channelopathies and can be debilitating or lethal 38,39 ADPKD is one of the most common life-threating monogenetic disorders and we have directly demonstrated that five variants impair the polycystin-2 channel function in the primary cilia membrane. Thus, the forms of ADPKD caused by PKD2 variants support it's categorization as a channelopathy. In mouse models of ADPKD, both overexpression and ablation of PKD2 can drive the phenotype in mice^{23,40}. While most large insertion and truncation variants likely cause a loss of function, the impact of missense variants is uncertain⁴¹. In our study, we focused on one location in polycystin-2 finger 1, where variants destabilize the TOP domain structure. The functional impact is a loss of channel function by reducing the channels' open probability via a shift in voltage dependence to positive potentials. Importantly, these channels are still “available” in the cilia membrane, and presents a rationale for the design of gating modifying drugs that target polycystin-2. Indeed, gating modulation is common mechanism of action in many prototypic therapeutic drugs that target ion channels^{42,43}. Our study demonstrates that destabilization of the TOP domain affects channel gating. Therefore, besides gating modifiers which target the VSD, theoretically, drugs could be developed to stabilize the TOP domain to achieve the same therapeutic effect. Since the TOP domain is a novel extracellular structural feature of polycystins, it may represent a unique receptor site to achieve drug target specificity. Other missense variants found in the VSD, pore and cytoplasmic domains may have drastically different consequence on polycystin-2 activity. Understanding these mechanistic differences from patients with unique variants may provide a rationale for the development of personalized medicine for the treatment of ADPKD.

The TOP domain's molecular regulation of polycystin-2. On the outset of this work, we proposed that variants in finger 1 might alter polycystin-2 activity by impairing channel opening, or assembly and trafficking to the primary cilia. Our study clearly demonstrates that these variants disrupt the stability of the TOP domain and impair channel gating without impacting channel cilia localization. Disrupting the disulfide interaction within finger 1-either by mutations or chemical reduction-results in a shift in the voltage dependence of polycystins (polycystin-2 and polycystin-2L1), enhancing the closure rate and doubling the amount of free energy required to open channels. This result prompts us to ask: What is the mechanistic model for how the TOP domain controls channel opening? We propose that the TOP domain may structurally gate this channel in one of two ways. First, it may serve as a fixed point from which the transmembrane portions of the channel move in response to changes in membrane potential and it transfers the motion to open the channel gate(s). In this model, the TOP domain-VSD interaction is structurally reminiscent of how spider toxins engage the VSD of voltage-gated sodium and potassium channels, restricting motion of the VSD and trapping them in conducting or non-conducting states^{44,45}. Alternatively, the TOP domain itself may move in response to activation of the VSD, transferring the motion of the VSD to the opening of the channel's proposed upper gate. Both gating model appears to be structurally plausible, and additional biophysical studies are required to determine which mechanism is most valid.

Implications for ADPKD classification as a ciliopathy. Nearly all gene variants implicated in inherited human cystic kidney diseases impact proteins that localize to the primary cilium or basal body and are usually accompanied by abnormal ciliary signaling^{46,47}. These diseases are categorized as renal ciliopathies due to their phenotypic convergence and subcellular localization of the impacted protein. Although not conclusive, ADPKD is commonly categorized as a renal ciliopathy. In this manuscript, we measured the biophysical dysregulation caused by polycystin-2 mutations associated with ADPKD directly from the primary cilia-which support the classification of ADPKD as a ciliopathy. This view is supported by two independent electrophysiology studies which measured polycystin-2's Ca²⁺ conductance in collecting duct primary cilia. Thus, Ca²⁺ dysregulation in the cilia is likely a direct consequence in ADPKD^{12,13}. Cells deficient in PKD2 are reported to have aberrant cytoplasmic calcium, a common signaling mechanism reported for other channelopathies^{48,49}. However, the primary cilium has its own resting Ca²⁺ concentration (580 nM) and changes in ciliary Ca²⁺ is demonstrably restricted to this compartment⁵⁰. In a recent study, flooding the ciliary compartment with millimolar Ca²⁺ did not affect levels found in the cytoplasm⁵¹. This result is supported by volumetric comparisons of cilia and the cell body, where the cilioplasmic volume of Ca²⁺ (<1 fL) is too small to alter the global cytoplasmic Ca²⁺ concentration (2-5 μL)⁵². This might explain why no difference in resting cytoplasmic Ca²⁺ was observed when PKD2 was overexpressed or genetically ablated in our HEK cells. Because of their local enrichment in the cilia, we propose that PKD2-mediated Ca²⁺-dependent signaling initially activates ciliary and periciliary proteins, such as adenylyl cyclase and PKA, which in turn regulates effectors of the Hedgehog pathway in the cell^{53,54}. It is noteworthy that other ciliopathies that impair brain development (such as Joubert syndrome, Bardet-Biedl syndrome and Alstrom Syndrome) are often caused by gene variants which encode for downstream Ca²⁺-signaling second messengers and commonly share polycystic kidney disease as a comorbidity^{36,79,80}. It is possible that aberrant cilia-to-cell signaling downstream of ciliary Ca²⁺ is a unifying mechanism for renal and non-renal ciliopathies. If this proves valid, future work should address which cilia effectors are involved and how downstream signaling pathways within the cytoplasm are responsible for the cystic kidney phenotype for ADPKD and other renal ciliopathies.

Polycystin-2 function in other membranes. In addition to primary cilia, homoterameric and heteromeric channels containing polycystin-2 are proposed to reside in plasma membranes, ER and mitochondria-associated ER membranes (MAMs)^{30,31,22,55,56} However, it is unknown which, or if all of these channel populations are involved in ADPKD progression. Conditional genetic ablation of PKD2 abolished the voltage-dependent, outwardly rectifying current measured from primary cilia of cyst-forming kidney collecting duct cells and demonstrated that these channels are functional in this organelle^{12,13}. However, cationic currents measured from the plasma membrane of these cells were unaltered. As initially reported, heterologous co-expression of PKD1 and PKD2 conducted a non-selective current with no voltage dependence (ohmic) in the plasma membrane of Chinese hamster ovary (CHO) cells³⁰. However, this work has proven difficult to reproduce, and subsequent work has drawn into question polycystin-2 function in the plasma membrane^{17,32}. It is possible that channels incorporating polycystin-1+polycystin-2 subunits have unique ion selectivity or have an undetermined gating mechanism which has prevented their detection and characterization¹⁴. But without determination of the basic gating properties of the putative heteromeric channel, its biological function in the cilia or plasma membranes remains an open question.

In this study, we examined InsPR3-mediated Ca²⁺ store release but did not observe differences when PKD2 was genetically ablated or overexpressed in HEK cells. This result contrasts with the polycystin-mediated Ca²⁺ store release reported in vascular smooth muscle cells and kidney collecting duct cells, and suggest that HEK cells are not a good model for these studies^{22,55,56} Gene expression differences between our cell lines and native cells may explain this divergence in results. The single channel properties of polycystin-2 measured from the ER and primary cilia membranes contrast significantly^{13,22} Polycystin-2 Ca²⁺ conductance measured from the primary cilium (4 pS) is smaller than those measured from ER reconstitutions (95 pS). Here, membrane composition and channel protein associations may account for these differences. For example, polycystin-2 reportedly forms complexes with resident ER proteins, including InsPR3⁵⁷⁵⁸. In addition, the ER and primary cilia membranes have different phosphoinositide and sphingomyelin compositions^{59,60}. The primary cilia membrane has been shown to contain a high level of phosphatidylinositol-4-phosphate established by inositol polyphosphate-5-phosphatase E, which localizes to the base of the cilium, whereas the ER membrane is primarily comprised of phosphoinsitide^{61,62}. Given that phospholipids have been shown to modulate channel function of other TRP channels, it is possible that lipid differences may modulate in polycystins channels as well^{63,64}. The reduced charge of phospholipids in the ER may modulate polycystin-2 gating, as implicated by the lipid occupancy sites found between the VSD and PD of the “multi-ion state” polycystin-2 structure¹⁵. Thus, polycystin gating behaviors reported from various organelles may have unique characteristics due to differences in membrane lipid compositions.

Redox regulation of polycystin-2 through the TOP domain disulfide bond. In this article, we demonstrate that conductance of polycystin-2 and polycystin-2L1 is regulated by redox potential. What is the physiological relevance for this observation? The proposed intercellular and primary cilia membrane pools of polycystin-2 channels in the kidney are in different redox environments. The lumen of the kidney tubule is an oxidizing environment (oxidizing potential E₀=250-300 mV)—partly established by the high levels (250-500 mM) of uric acid produced by the glomerulus⁶⁵—whereas the lumen of ER (reducing potential E₀=−170 mV)^{66,67} and cytosol (E₀=−290 mV)⁶⁸ are in a highly reducing environment, largely due to concentrated GSH (˜10 mM) production catalyzed by GSH reductase^{69,70}. We demonstrate that polycystin-2 function is abolished by external application of GSH and TCEP (E₀=−240 and −290 mV, respectively)^{71,72}. This effect is caused by chemically reducing the C331-C344 finger 1 disulfide bond, which keeps the channel closed. This interaction is conserved in the related polycystin-2L1 channel found in non-renal cilia, and may represent a defining feature of the polycystin subfamily of TRP channels. The redox-inhibition feature might be advantageous to attenuate polycystin channel function intracellularly until it is trafficked to the ciliary membrane. Here, nascent channels would remain closed by GSH reduction of the finger 1 disulfide bond in the cytosolic compartment until they are trafficked to the cilium. Recently, endogenous polycystin-2 has been identified in MAMs and is proposed to facilitate Ca²⁺ transfer between ER and mitochondria⁷³. Mitochondria respiratory bursts may impact the local redox potential in MAMs through temporal calcium-induced production of reactive oxidation species⁷⁴. Future work assessing redox effects on reconstituted polycystin-2 from intracellular membranes would be helpful in determining how this population is physiologically relevant and if it is involved in ADPKD progression.

Methods

Generation of HEK PKD2^{Null} cells line and the stable expression PKD2 variants. To generate the HEK PKD2^{Null} cell lines, we used a CRISPR/Cas9 gene editing kit available from Addgene (kit 1000000055). HEK 293 cells were electro-transfected with sgRNAs (caccgAGACACCCCCGTGTCCAAAA and aaacTTTTGGACACGGGGGTGTCTc) with the All-in-one Cas9 plasmid. Sequence analysis confirming the homozygous knockout was performed after puromycin selection. Cells generated from single cell clones were selected after 4 weeks of expansion in a 96-wells plate. A positive HEK PKD2^{Null} clone was verified after extracting the genomic DNA and polymerase chain reaction amplification of the STOP codons with forward (AGCCTCAGGGCACAGAACAG) and reverse (CCACACTGCCCTTCATTGGC) primers. To generate the WT and variant PKD2 GFP or mCherry C-terminally tagged variant cell lines, the hPKD gene was subcloned into lentiviral pLVX-mcherry-N1 (Clontech) or pLVX-GFP-N1 (Clontech) vector using the Gibson assembly method. A linker encoding for six glycine residues was added between the PKD2 gene and the C-terminal tags, and missense variants were generated using standard, site-directed mutagenesis. The third-generation lentiviral packaging plasmids used for stable expression contained: pMDLg/pRRE (Addgene), reverse transcriptase pRSV-Rev (Addgene), and envelope expressing plasmid pMD2.G (Addgene). LentiX-293T cells (Takara) were transfected with polyethylineimine (Polysciences) at a 4:1:1:1 ratio of the transgene and viral packaging constructs. Supernatants were collected 48 and 72 hours post transfection and filtered through a 0.45 μm syringe filter. Lentiviral supernatant was concentrated 100 times using 1 volume of PEG-it (System Biosciences) virus precipitation solution and 4 volume of lentivirus-containing supernatant. The PEG-it and supernatant mixture were kept at 4 degrees for 24 hours and centrifuged at 1500 rpm for 30 minutes. The pellet containing lentivirus was resuspended with 1/100th volume of PBS of the original supernatant volume. HEK cells were infected with the lentivirus supernatant; PKD2-GFP or PKD2-mCherry expression was selected using culture media containing puromycin (2 μg/ml) for 30 to 90 days. Cells were then fluorescence-activated cell sorted (BD FacsMelody) at 5000 to 10,000 counts per minute to enrich for the transgene expression. Stable cell lines were cultured in Dulbecco's modified essential medium (DMEM) supplemented with 10% fetal bovine serum (FBS) and 100 units/ml penicillin, 100 units/ml streptomycin and 1 μg/ml puromycin selection antibiotic.

Electrophysiology. Ciliary ion currents were recorded using borosilicate glass electrodes polished to resistances of 14-22 MΩ using the cilium patch method previously described³². Whole cell electrodes used to measure poly cystin-2L1 currents were fire polished to 1.5-4 MΩ resistances. Unless otherwise stated, whole cilia ionic currents were recorded in symmetrical [Na⁺]. The pipette standard internal solution contained (in mM): 90 NaMES, 10 NaCl, 10 HEPES, 10 Na4-BAPTA (Glycine, N, N′-[1,2-ethanediylbis(oxy-2,1-phenylene)]bis[N-(carboxymethyl)]-,tetrasodium); pH was adjusted to 7.3 using NaOH. Standard bath solution contained 140 NaCl, 10 HEPES, 1.8 CaCl₂); pH 7.4. All solutions were osmotically balanced to 295 (±6) mOsm with mannitol. Extracellular solutions containing TCEP and GSH were used within six hours of formulation. Data were collected using an Axopatch 200B patch clamp amplifier, Digidata 1440A, and pClamp 10 software. Whole-cilium and excised patch currents were digitized at 25 kHz and low-pass filtered at 10 kHz. In the whole cilium configuration, the holding potential was −60 mV and then depolarized using a 400 ms voltage ramp from −100 mV to 100 mV. The whole cell recordings of polycystin-2L1 currents were either activated by the same ramp protocol or by 100 ms depolarizations to 100 mV from a −100 mV holding potential. External conditions were controlled using a Warner Perfusion Fast-Step (SF-77B) system in which the patched cilia and electrode were held in the perfusate stream. Data were analyzed by Igor Pro 7.00 (Wavemetrics, Lake Oswego, Oreg.). The polycystin-2 open probability and the polycystin-2L1 tail current-voltage relationships were fit to a Boltzmann function, f(x)=1/(1+exp[V−V1/2]/k) to estimate voltage of half-maximal activation of current (V_{1/2}).

Intracellular calcium measurements using Fura-2. Carbachol-mediated intracellular calcium responses were measured from HEK cells expressing endogenous MIR or from cells overexpressing the M1R-mCherry plasmid delivered using the BacMam expression system (Montana Molecular). After 24 hours, HEK cells with indicated PKD2 genotypes were seeded onto glass bottom plates (MatTek Corporation) pre-coated with poly-L-lysine (Sigma) for 20 hours. The cells were incubated for 1 hour at 37° C. in complete medium after loading with 2 μg of Fura-2/AM (Invitrogen). The cells were then washed with and stored for 15 minutes in Tyrode's solution (in mM): 140 NaCl, 4 KCl, 2 MgCl2, 2 CaCl₂), 10 Glucose, 10 HEPES. Cells were adjusted in pH to 7.4 with NaOH and osmolarity to 300 mOs with D-mannitol. Cells were placed under an inverted wide-field microscope equipped with 20× objective lens (Olympus IX81) and the stage temperature was held at 37 C (Tokai Hit). Images of fura-2 fluorescence remission at 520 nm were captured every 2 seconds during excitation at 320 nm (Ca²⁺ free) and 340 nm (Ca²⁺ bound). Images were acquired and analyzed using SlideBook (Intelligent imaging solutions) which synchronizes the filter wheel changer (Lambda 10-3, Sutter Instrument) and the camera (ImagEMX2, Hamamatsu). After 40 seconds in the control solution, 3 μM or 50 μM carbachol was added to the imaging chamber via exchange at rate of 2-4 ml/minute. Data analysis was performed on Slidebook. Ten to 20 cells were selected from the field of view and their ROIs of the emission fluorescence from both wavelengths were recorded after subtracting the background fluorescence level; these data were reported as 340:380 nm ratio. This ratio was averaged at the start (for resting level) and the response was reported after carbachol administration (for maximal response). At least four replicates (N) were performed for each variant. The emission ratios were converted into a measurement of free cytosolic Ca²⁺ concentration using the following equation:

$$[ {Ca}^{2 +} ] = {K}_{d} \cdot \frac{{F}_{max}}{{F}_{min}} \cdot \frac{( R - {R}_{min} )}{( {R}_{max} - R )}$$

where Rₘᵢₙ and Rₘₐₓ is the minimum and maximum experimental emission 340:380 nm ratio, respectively, Fₘₐₓ and Fₘᵢₙ is the 380 nm maximum and minimum fluorescence emission signal at nominal free Ca²⁺ (measured in Tyrode's solution without CaCl₂), but with 5 mM EGTA and 20 μM ionomycin added); Fₘᵢₙ and Rₘₐₓ are similar, except at maximal Ca²⁺ level (measured in Tyrode's solution with 10 mM CaCl₂) added and 20 μM ionomycin); The Fura-2 and Ca²⁺ dissociation constant (Kd) was 224 nM, as determined experimentally using a previously described method⁷⁵.

Immunocytochemistry, confocal microscopy and SIM Cells were fixed with 4% paraformaldehyde (PFA), permeabilized with 0.2% Triton X-100, and blocked by 10% bovine serum albumin in PBS. Cells and tissue were mounted on glass slides and treated with Fluoshield from Sigma-Aldrich (St. Louis Mo.). Detailed information on the type and concentration of primary and secondary antibodies used in this study are listed in FIG. 16. Confocal images were obtained using an inverted Nikon A1 with a 60× silicon oil immersion, 1.3 N.A. objective. Super resolution images using the SIM method were captured under 100× magnification using the Nikon Structured Illumination Super-Resolution Microscope (N-SIM) with piezo stepping. Confocal and SIM images were further processed with FIJI ImageJ (National Institutes of Health).

Cryo-EMdata acquisition. Each 2.5 μl of PKD2 sample at ˜1.5 mg/ml was applied to glow-discharged UltrAuFoil 1.2/1.3 holey 300 mesh gold grids. Grids were plunge frozen in liquid ethane using a Vitrobot Mark III (FEI) set to 4° C., 85% relative humidity, 20 seconds wait time, −1 mm offset, and 2.5 seconds blotting time. Data were collected on a Krios (FEI) operating at 300 kV equipped with the K2 Summit direct electron detector at Yale University and National Center for CryoEM Access and Training (NCCAT). Images were recorded using SerialEM at Yale or Leginon at NCCAT, with a defocus range between −1.5 to −2.5 μm^{76,77}. Specifically, we recorded movies in super-resolution counting mode at a magnification of 47,619×, which corresponds to a physical pixel size of 1.05 Å. The data were collected at a dose rate of 1.4 e⁻/Å²/frame with a total exposure of 40 frames, giving a total dose of 59 e⁻/Λ².

Image processing, 3D reconstruction, and model building. Movie frames were aligned, dose weighted, and then summed into a single micrograph using MotionCor2. Contrast transfer function parameters for micrographs were determined using the program CTFFIND4^{78,79}. An estimated 2,000 particles were manually boxed out in RELION to generate initial 2D averages, which were then used as templates to automatically pick particles from all micrographs. ‘Junk’ particles (i.e., ice contamination and gold support) were manually rejected and all subsequent 2D and 3D processing steps were performed using RELION⁸⁰. Specifically, 501,566 particles were initially extracted from 2,143 micrographs and a round of 2D classification (followed by another round of 3D classification) was performed to reject bad particles from downstream analyses. This resulted in a final dataset of ˜74,302 particles that was subsequently used for 3D reconstruction. For 3D classification and refinement, the PKD2 structure (EMD-8354, low-pass filtered to 60 Å) was used as the starting model with C4 symmetry imposed. RELION auto-refinement with C4 symmetry imposed yielded a 3.78 Å resolution map without masking; removing amphipol belt and solvent with a soft mask improved the map to 3.24 Å resolution based on cutoff of gold standard FSC=0.143. The mask was generated in RELION using the relion_mask_create program against the summed two half maps with options that extend 3 pixels beyond a preset density threshold of 0.016 and produce a soft edge of 3 pixels. Local resolution was calculated by RELION. We also carried out reconstruction without imposing any symmetry during 3D classification and refinement steps, and all resulting maps exhibit a rough C4 symmetry except at some disordered loops and the amphipol belt surrounding the transmembrane region of the channels. Since the map calculated with C4 symmetry was better resolved, we used this map for model building and structural analyses. The map was sharpened with a b factor of −100 Å² for model building in Coot⁸¹. The model was then refined in real space using PHENIX, and assessed by Molprobity (FIG. 15)^{8,83}. The Fourier shell correlation (FSC) curve was calculated between the refined model versus summed half maps generated in RELION, and resolution was reported at a cutoff of FSC=0.5. UCSF Chimera was used to visualize and segment density maps, as well as to generate figures⁸⁴.

Thermal stability assay. Approximately 9 μg of purified human PKD2 channel proteins (WT or C331S) in 30 μl buffer composed of 20 mM HEPES 150 mM NaCl, 2 mM CaCl₂), 0.5 mM TCEP and 0.5 mM DDM (n-Dodecyl β-D-maltoside), at pH 7.4, were incubated at 4° C.-90° C. (4° C., room temperature, 30° C., 35.2° C., 39.3° C., 44.9° C., 49° C., 54° C., 60° C., 65.5° C., 69.4° C., 75° C., 79.3° C., 84° C., and 90° C.) for 10 minutes in a thermal cycler. To reduce the C331-C344 disulfide bond in the PKD2 channel, 10 mM GSH was added to all the buffers during purification and thermal stability assay. The treated channel samples were then diluted 10 times with the same buffer followed by centrifugation for 30 min at 40,000 rpm. 30 μl of each cleared channel samples were separated on an analytical size exclusion column (Superose™ 6 5/150 GL, GE Healthcare) at 0.3 ml/min flow rate. Proteins were detected by Tryptophan fluorescence. The FSEC-based thermostability experiments were performed in triplicates for each temperature point. The integrated area of the channel tetramer peak at different temperature points is normalized to that at 4° C. to generate the thermal stability plot.

Statistical analysis. Statistical comparisons were made using two-tailed Student's t-tests using OriginPro software (OriginLab, Northampton Mass.). Experimental values are reported as the mean S.E.M. unless otherwise stated. Differences in mean values were considered significant at p<0.05. All of our results were normally distributed per Shapiro-Wilk Test. The results from FIG. 2, S3 and S5 are parametric for all data sets with a P value threshold of 0.1 to reject normalcy.

Data availability Statement: All cDNA constructs, and cell lines used in this study will be available upon request by PGD unless the item(s) are already deposited and available through addgene (www.addgene.org/). The polycytsin-2 C331S variant molecular structure coordinates derived from the cryo-EM data sets will be available through the RCSB Protein Data Bank repository (www.rcsb.org/). The data that support the findings of this study are available from the corresponding author, PGD and EC, upon reasonable request.

### REFERENCES

1 Torres, V. E., Harris, P. C. & Pirson, Y. Autosomal dominant polycystic kidney disease. Lancet 369, 1287-1301, doi:10.1016/S0140-6736(07)60601-1 (2007).
2 Gabow, P. A. Autosomal dominant polycystic kidney disease. N Engl J Med 329, 332-342, doi:10.1056/NEJM199307293290508 (1993).
3 Mochizuki, T. et al. PKD2, a gene for polycystic kidney disease that encodes an integral membrane protein. Science 272, 1339-1342 (1996).
4 Hughes, J. et al. The polycystic kidney disease 1 (PKD1) gene encodes a novel protein with multiple cell recognition domains. Nat Genet 10, 151-160, doi:10.1038/ng0695-151 (1995).
5 Koptides, M., Hadjimichael, C., Koupepidou, P., Pierides, A. & Constantinou Deltas, C. Germinal and somatic mutations in the PKD2 gene of renal cysts in autosomal dominant polycystic kidney disease. Human molecular genetics 8, 509-513 (1999).
6 Bergmann, C. et al. Polycystic kidney disease. Nature reviews. Disease primers 4, 50, doi:10.1038/s41572-018-0047-y (2018).
7 Venkatachalam, K. & Montell, C. TRP channels. Annual review of biochemistry 76, 387-417, doi:10.1146/annurev.biochem.75.103004.142819 (2007).
8 Brasier, J. L. & Henske, E. P. Loss of the polycystic kidney disease (PKD1) region of chromosome 16p13 in renal cyst cells supports a loss-of-function model for cyst pathogenesis. J Clin Invest 99, 194-199, doi:10.1172/JCI119147 (1997).
9 Bangs, F. & Anderson, K. V. Primary Cilia and Mammalian Hedgehog Signaling. Cold Spring Harb Perspect Biol 9, doi:10.1101/cshperspect.a028175 (2017).
10 Drummond, L. A. Cilia functions in development. Curr Opin Cell Biol 24, 24-30, doi:10.1016/j.ceb.2011.12.007 (2012).
11 Oh, E. C. & Katsanis, N. Cilia in vertebrate development and disease. Development 139, 443-448, doi:10.1242/dev.050054 (2012).
12 Kleene, S. J. & Kleene, N. K. The native TRPP2-dependent channel of murine renal primary cilia. Am J Physiol Renal Physiol 312, F96-F108, doi:10.1152/ajprenal.00272.2016 (2017).
13 Liu, X. et al. Polycystin-2 is an essential ion channel subunit in the primary cilium of the renal collecting duct epithelium. eLife 7, doi:10.7554/eLife.33183 (2018).
14 Su, Q. et al. Structure of the human PKD1-PKD2 complex. Science 361, doi:10.1126/science.aat9819 (2018).
15 Wilkes, M. et al. Molecular insights into lipid-assisted Ca(2+) regulation of the TRP channel Polycystin-2. Nat Struct Mol Biol 24, 123-130, doi:10.1038/nsmb.3357 (2017).
16 Grieben, M. et al. Structure of the polycystic kidney disease TRP channel Polycystin-2 (PC2). Nat Struct Mol Biol 24, 114-122, doi:10.1038/nsmb.3343 (2017).
17 Shen, P. S. et al. The Structure of the Polycystic Kidney Disease Channel PKD2 in Lipid Nanodiscs. Cell 167, 763-773 e711, doi:10.1016/j.cell.2016.09.048 (2016).
18 Wang, Z. et al. The ion channel function of polycystin-1 in the poly cystin-1/poly cystin-2 complex. EMBO reports, e48336, doi:10.15252/embr.201948336 (2019).
19 Rossetti, S. et al. Comprehensive molecular diagnostics in autosomal dominant polycystic kidney disease. J Am Soc Nephrol 18, 2143-2160, doi:10.1681/ASN.2006121387 (2007).
20 Hateboer, N. et al. Comparison of phenotypes of polycystic kidney disease types 1 and 2. European PKD1-PKD2 Study Group. Lancet 353, 103-107, doi:10.1016/s0140-6736(98)03495-3 (1999).
21 Li, A. et al. Human polycystin-2 transgene dose-dependently rescues ADPKD phenotypes in Pkd2 mutant mice. Am J Pathol 185, 2843-2860, doi:10.1016/j.ajpath.2015.06.014 (2015).
22 Koulen, P. et al. Polycystin-2 is an intracellular calcium release channel. Nat Cell Biol 4, 191-197, doi:10.1038/ncb754 (2002).
23 Park, E. Y. et al. Cyst formation in kidney via B-Raf signaling in the PKD2 transgenic mice. J Biol Chem 284, 7214-7222, doi:10.1074/jbc.M805890200 (2009).
24 Burtey, S. et al. Overexpression of PKD2 in the mouse is associated with renal tubulopathy. Nephrol Dial Transplant 23, 1157-1165, doi:10.1093/ndt/gfm763 (2008).
25 Gout, A. M., Martin, N. C., Brown, A. F. & Ravine, D. PKDB: Polycystic Kidney Disease Mutation Database—a gene variant database for autosomal dominant polycystic kidney disease. Hum Mutat 28, 654-659, doi:10.1002/humu.20474 (2007).
26 Brohawn, S. G., del Marmol, J. & MacKinnon, R. Crystal structure of the human K2P TRAAK, a lipid- and mechano-sensitive K+ ion channel. Science 335, 436-441, doi:10.1126/science.1213808 (2012).
27 Yelshanskaya, M. V., Mesbahi-Vasey, S., Kurnikova, M. G. & Sobolevsky, A. I. Role of the Ion Channel Extracellular Collar in AMPA Receptor Gating. Sci Rep 7, 1050, doi:10.1038/s41598-017-01146-z (2017).
28 Lee, C. et al. The lysosomal potassium channel TMEM175 adopts a novel tetrameric architecture. Nature 547, 472-475, doi:10.1038/nature23269 (2017).
29 Hamel, V. et al. Correlative multicolor 3D SIM and STORM microscopy. Biomed Opt Express 5, 3326-3336, doi:10.1364/BOE.5.003326 (2014).
30 Prodromou, N. V. et al. Heat shock induces rapid resorption of primary cilia. Journal of cell science 125, 4297-4305, doi:10.1242/jcs.100545 (2012).
31 Plotnikova, O. V., Pugacheva, E. N. & Golemis, E. A. Primary cilia and the cell cycle. Methods in cell biology 94, 137-160, doi:10.1016/S0091-679X(08)94007-3 (2009).
32 DeCaen, P. G., Delling, M., Vien, T. N. & Clapham, D. E. Direct recording and molecular identification of the calcium channel of primary cilia. Nature 504, 315-318, doi:10.1038/nature12832 (2013).
33 Hulse, R. E., Li, Z., Huang, R. K., Zhang, J. & Clapham, D. E. Cryo-EM structure of the polycystin 2-11 ion channel. eLife 7, doi:10.7554/eLife.36931 (2018).
34 Su, Q. et al. Cryo-EM structure of the polycystic kidney disease-like channel PKD2L1. Nature communications 9, 1192, doi:10.1038/s41467-018-03606-0 (2018).
35 Arif Pavel, M. et al. Function and regulation of TRPP2 ion channel revealed by a gain-of-function mutant. Proc Natl Acad Sci USA 113, E2363-2372, doi:10.1073/pnas.1517066113 (2016).
36 Hille, B. Ion channels of excitable membranes. 3rd edn, (Sinauer, 2001).
37 Kass, R. S. The channelopathies: novel insights into molecular and genetic mechanisms of human disease. J Clin Invest 115, 1986-1989, doi:10.1172/JCI26011 (2005).
38 Imbrici, P. et al. Therapeutic Approaches to Genetic Ion Channelopathies and Perspectives in Drug Discovery. Frontiers in pharmacology 7, 121, doi:10.3389/fphar.2016.00121 (2016).
39 George, A. L., Jr. Inherited disorders of voltage-gated sodium channels. The Journal of clinical investigation 115, 1990-1999, doi:10.1172/JCI25505 (2005).
40 Wu, G. et al. Somatic inactivation of Pkd2 results in polycystic kidney disease. Cell 93, 177-188 (1998).
41 Rossetti, S. et al. Incompletely penetrant PKD1 alleles suggest a role for gene dosage in cyst initiation in polycystic kidney disease. Kidney international 75, 848-855, doi:10.1038/ki.2008.686 (2009).
42 Wulff, H., Castle, N. A. & Pardo, L. A. Voltage-gated potassium channels as therapeutic targets. Nat Rev Drug Discov 8, 982-1001, doi:10.1038/nrd2983 (2009).
43 de Lera Ruiz, M. & Kraus, R. L. Voltage-Gated Sodium Channels: Structure, Function, Pharmacology, and Clinical Indications. J Med Chem 58, 7093-7118, doi:10.1021/jm501981g (2015).
44 Swartz, K. J. & MacKinnon, R. Mapping the receptor site for hanatoxin, a gating modifier of voltage-dependent K+ channels. Neuron 18, 675-682 (1997).
45 Bosmans, F. & Swartz, K. J. Targeting voltage sensors in sodium channels with spider toxins. Trends in pharmacological sciences 31, 175-182, doi:10.1016/j.tips.2009.12.007 (2010).
46 Hildebrandt, F., Benzing, T. & Katsanis, N. Ciliopathies. N Engl J Med 364, 1533-1543, doi:10.1056/NEJMra1010172 (2011).
47 Arts, H. H. & Knoers, N. V. Current insights into renal ciliopathies: what can genetics teach us? Pediatric nephrology 28, 863-874, doi:10.1007/s00467-012-2259-9 (2013).
48 Seitter, H. & Koschak, A. Relevance of tissue specific subunit expression in channelopathies. Neuropharmacology 132, 58-70, doi:10.1016/j.neuropharm.2017.06.029 (2018).
49 Rubio-Moscardo, F. et al. Rare variants in calcium homeostasis modulator 1 (CALHM1) found in early onset Alzheimer's disease patients alter calcium homeostasis. PloS one 8, e74203, doi:10.1371/journal.pone.0074203 (2013).
50 Delling, M., DeCaen, P. G., Doerner, J. F., Febvay, S. & Clapham, D. E. Primary cilia are specialized calcium signalling organelles. Nature 504, 311-314, doi:10.1038/nature12833 (2013).
51 Delling, M. et al. Primary cilia are not calcium-responsive mechanosensors. Nature 531, 656-660, doi:10.1038/naturel7426 (2016).
52 Pablo, J. L., DeCaen, P. G. & Clapham, D. E. Progress in ciliary ion channel physiology. The Journal of general physiology 149, 37-47, doi:10.1085/jgp.201611696 (2017).
53 Tukachinsky, H., Lopez, L. V. & Salic, A. A mechanism for vertebrate Hedgehog signaling: recruitment to cilia and dissociation of SuFu-Gli protein complexes. The Journal of cell biology 191, 415-428, doi:10.1083/jcb.201004108 (2010).
54 Vuolo, L., Herrera, A., Torroba, B., Menendez, A. & Pons, S. Ciliary adenylyl cyclases control the Hedgehog pathway. Journal of cell science 128, 2928-2937, doi:10.1242/jcs.172635 (2015).
55 Vassilev, P. M. et al. Polycystin-2 is a novel cation channel implicated in defective intracellular Ca(2+) homeostasis in polycystic kidney disease. Biochem Biophys Res Commun 282, 341-350, doi:10.1006/bbrc.2001.4554 (2001).
56 Qian, Q. et al. Pkd2 haploinsufficiency alters intracellular calcium regulation in vascular smooth muscle cells. Hum Mol Genet 12, 1875-1880 (2003).
57 Sammels, E. et al. Polycystin-2 activation by inositol 1,4,5-trisphosphate-induced Ca2+ release requires its direct association with the inositol 1,4,5-trisphosphate receptor in a signaling microdomain. The Journal of biological chemistry 285, 18794-18805, doi:10.1074/jbc.M109.090662 (2010).
58 Li, Y., Wright, J. M., Qian, F., Germino, G. G. & Guggino, W. B. Polycystin 2 interacts with type I inositol 1,4,5-trisphosphate receptor to modulate intracellular Ca2+ signaling. J Biol Chem 280, 41298-41306, doi:10.1074/jbc.M510082200 (2005).
59 Raychaudhuri, S., Im, Y. J., Hurley, J. H. & Prinz, W. A. Nonvesicular sterol movement from plasma membrane to ER requires oxysterol-binding protein-related proteins and phosphoinositides. The Journal of cell biology 173, 107-119, doi:10.1083/jcb.200510084 (2006).
60 van Meer, G., Voelker, D. R. & Feigenson, G. W. Membrane lipids: where they are and how they behave. Nature reviews. Molecular cell biology 9, 112-124, doi:10.1038/nrm2330 (2008).
61 Phua, S. C. et al. Dynamic Remodeling of Membrane Composition Drives Cell Cycle through Primary Cilia Excision. Cell 168, 264-279 e215, doi:10.1016/j.cell.2016.12.032 (2017).
62 Garcia-Gonzalo, F. R. et al. Phosphoinositides Regulate Ciliary Protein Trafficking to Modulate Hedgehog Signaling. Dev Cell 34, 400-409, doi:10.1016/j.devcel.2015.08.001 (2015).
63 Laub, K. R. et al. Comparing ion conductance recordings of synthetic lipid bilayers with cell membranes containing TRP channels. Biochimica et biophysica acta 1818, 1123-1134, doi:10.1016/j.bbamem.2012.01.014 (2012).
64 Badheka, D., Borbiro, I. & Rohacs, T. Transient receptor potential melastatin 3 is a phosphoinositide-dependent ion channel. The Journal of general physiology 146, 65-77, doi:10.1085/jgp.201411336 (2015).
65 Giebisch, G. Measurements of electrical potentials and ion fluxes on single renal tubules. Circulation 21, 879-891 (1960).
66 Hwang, C., Sinskey, A. J. & Lodish, H. F. Oxidized redox state of glutathione in the endoplasmic reticulum. Science 257, 1496-1502 (1992).
67 Birk, J. et al. Endoplasmic reticulum: reduced and oxidized glutathione revisited. Journal of cell science 126, 1604-1617, doi:10.1242/jcs.117218 (2013).
68 Grant, C. M., MacIver, F. H. & Dawes, I. W. Glutathione is an essential metabolite required for resistance to oxidative stress in the yeast Saccharomyces cerevisiae. Current genetics 29, 511-515 (1996).
69 Go, Y. M. & Jones, D. P. Redox compartmentalization in eukaryotic cells. Biochimica et biophysica acta 1780, 1273-1290, doi:10.1016/j.bbagen.2008.01.011 (2008).
70 Morgan, B., Sobotta, M. C. & Dick, T. P. Measuring E(GSH) and H₂O₂ with roGFP2-based redox probes. Free radical biology & medicine 51, 1943-1951, doi:10.1016/j.freeradbiomed.2011.08.035 (2011).
71 Pullela, P. K., Chiku, T., Carvan, M. J., 3rd & Sem, D. S. Fluorescence-based detection of thiols in vitro and in vivo using dithiol probes. Analytical biochemistry 352, 265-273, doi:10.1016/j.ab.2006.01.047 (2006).
72 Peng, L. et al. Effects of metal ions and disulfide bonds on the activity of phosphodiesterase from Trimeresurus stejnegeri venom. Metallomics: integrated biometal science 5, 920-927, doi:10.1039/c3mt00031a (2013).
73 Kuo, I. Y. et al. Polycystin 2 regulates mitochondrial Ca(2+) signaling, bioenergetics, and dynamics through mitofusin 2. Science signaling 12, doi:10.1126/scisignal.aat7397 (2019).
74 Chaube, R. & Werstuck, G. H. Mitochondrial ROS versus ER ROS: Which Comes First in Myocardial Calcium Dysregulation? Frontiers in cardiovascular medicine 3, 36, doi:10.3389/fcvm.2016.00036 (2016).
75 Grynkiewicz, G., Poenie, M. & Tsien, R. Y. A new generation of Ca2+ indicators with greatly improved fluorescence properties. The Journal of biological chemistry 260, 3440-3450 (1985).
76 Potter, C. S. et al. Leginon: a system for fully automated acquisition of 1000 electron micrographs a day. Ultramicroscopy 77, 153-161 (1999).
77 Mastronarde, D. N. Automated electron microscope tomography using robust prediction of specimen movements. Journal of structural biology 152, 36-51, doi:10.1016/j.jsb.2005.07.007 (2005).
78 Zheng, S. Q. et al. MotionCor2: anisotropic correction of beam-induced motion for improved cryo-electron microscopy. Nature methods 14, 331-332, doi:10.1038/nmeth.4193 (2017).
79 Rohou, A. & Grigorieff, N. CTFFIND4: Fast and accurate defocus estimation from electron micrographs. Journal of structural biology 192, 216-221, doi:10.1016/j.jsb.2015.08.008 (2015).
80 Scheres, S. H. RELION: implementation of a Bayesian approach to cryo-EM structure determination. Journal of structural biology 180, 519-530, doi:10.1016/j.jsb.2012.09.006 (2012).
81 Emsley, P., Lohkamp, B., Scott, W. G. & Cowtan, K. Features and development of Coot. Acta crystallographica. Section D, Biological crystallography 66, 486-501, doi:10.1107/S0907444910007493 (2010).
82 Echols, N. et al. Graphical tools for macromolecular crystallography in PHENIX. Journal of applied crystallography 45, 581-586, doi:10.1107/S0021889812017293 (2012).
83 Williams, C. J. et al. MolProbity: More and better reference data for improved all-atom structure validation. Protein science: a publication of the Protein Society 27, 293-315, doi:10.1002/pro.3330 (2018).
84 Pettersen, E. F. et al. UCSF Chimera—a visualization system for exploratory research and analysis. Journal of computational chemistry 25, 1605-1612, doi:10.1002/jcc.20084 (2004).

### Example 2-Disrupting Polycystin-2 EF Hand Ca²⁺ Affinity does not Alter Channel Function or Contribute to Polycystic Kidney Disease

Reference is made to Vien et al., “Disrupting polycystin-2 EF hand Ca2+ affinity does not alter channel function or contribute to polycystic kidney disease,” J. Cell Science (2020); Dec. 24; 133(24); 1-10, the content of which is incorporated herein by reference in its entirety.

Introduction

PKD2 encodes for polycystin-2, a member of the polycystin subfamily of transient receptor potential ion channels (TRPP) (Venkatachalam and Montell, 2007). Previous work has established that polycystins form calcium-conducting channels in the primary cilia membrane of disparate tissues (DeCaen et al., 2013; Kleene and Kleene, 2012; Kleene and Kleene, 2017; Liu et al., 2018). Primary cilia are solitary projections that extend ˜5 microns from the apical side of cells. They are privileged cellular organelles comprised of less than 500 unique protein components and are found in all organ systems—including cells of the kidney nephron (Ostrowski et al., 2002; Pazour et al., 2005). Primary cilia house specific receptors and downstream effectors, which are capable of modulating morphogenic gene transcription responsible for left-right axis determination in developing vertebrate embryos (Braun and Hildebrandt, 2017; Mizuno et al., 2020; Nachury and Mick, 2019). Dysregulation of cilia- and centrosome-localized proteins results in ciliopathies-conditions that often affect specific organ systems but commonly share cystic kidney diseases as a comorbidity (Hildebrandt et al., 2011; Hildebrandt and Zhou, 2007). Variants in human PKD2 account for ˜15% of cases of autosomal dominant polycystic kidney disease (ADPKD) (Brasier and Henske, 1997; Grantham, 2001; Hughes et al., 1995; Mochizuki et al., 1996)—a common ciliopathy that is characterized by progressive cyst development which ultimately causes renal failure. ADPKD is proposed to be a recessive disease at the cellular level, where individuals inherit one variant polycystin allele and develop cysts after acquiring a second somatic mutation in the remaining allele (Koptides et al., 1999; Pei, 2001; Qian et al., 1997; Wu et al., 1998). Mouse models of attenuating Pkd2 expression by conditional genetic repression faithfully recapitulate the polycystic kidney phenotype (Happe and Peters, 2014; Ma et al., 2013; Wilson, 2008; Wu et al., 1998), whereas complete genetic deletion of Pkd2 causes embryonic lethality and kidney cyst development in utero (Menezes and Germino, 2013; Wu et al., 2000). These studies in mice suggest that most ADPKD-causing variants are loss of function, but our understanding of their mechanistic impact remains largely undetermined due to their subcellular localization.

The primary cilium is electrochemically discrete from the cytosol, having its own depolarized membrane potential and resting concentration of calcium (DeCaen et al., 2013; Delling et al., 2013). Analyzing polycystin-2 in primary cilia has opened opportunities to study its function under endogenous and heterologous expression conditions (Kleene and Kleene, 2017; Liu et al., 2018). Most recently, this methodology was used to determine that several disease-causing variants found in the human ADPKD population alter polycystin-2 gating without affecting its ciliary localization and tetramerization (Vien et al., 2020). Polycystin-2 forms a voltage-gated channel that opens at positive membrane potentials. Since the cilia resting membrane potential is approximately −17 mV, most channels are closed until internal Ca²⁺ is elevated to micromolar concentrations (Delling et al., 2013). Once elevated, the voltage required to open polycystin-2 becomes hypopolarized and channel closure becomes incomplete at negative membrane potentials (Kleene and Kleene, 2017; Liu et al., 2018). In this manuscript, we give the term ‘calcium-dependent modulation’ (CDM) to describe this form of polycystin-2 gating regulation. Importantly, this mechanism gives polycystin-2 gating its physiological relevance, allowing Ca²⁺ and monovalent ions to flow into the primary cilium at its resting membrane potential. Subsequently, prolonged and elevated intraciliary Ca²⁺ causes polycystin-2 channels to enter an irreversible, non-conducting state, through a process we have termed ‘calcium-dependent desensitization’ (CDD). This form of polycystin regulation was postulated to protect the cell from Ca²⁺ overload, by turning off the flow of calcium at the source within the ciliary compartment (DeCaen et al., 2013; DeCaen et al., 2016). While CDD has been attributed to calcium occupancy of the related polycystin-2L1 pore, the structural determinants responsible for CDD and CDM of polycystin-2 is unknown.

Besides primary cilia, polycystin-2 activity is reported in the membranes of intracellular organelles (Koulen et al., 2002; Kuo et al., 2019). Polycystin-2 functionally mimics the Ca²⁺ response from bonafide ER ion channels, so its contribution is difficult to distinguish from inositol trisphosphate (IP3R) and ryanodine receptor channel (RYR) mediated effects (Koulen et al., 2002; Qian et al., 2003; Vassilev et al., 2001). Measuring polycystin-2 in bilayers from reconstituted ER membranes is challenging, in part because it directly associates with IP3R (Santoso et al., 2011). Results from reconstitution and cytosolic calcium assays have demonstrated that ER localized polycystin-2 channels are responsive to intracellular calcium and that mutations that remove the entire C-terminal domain (CTD) or alter the EF hand abolish the channel's function entirely (Celic et al., 2012; Koulen et al., 2002). Partly based on this work, it was proposed that calcium occupancy of its C-terminal EF hand is requisite for polycystin-2 channel function and calcium-dependent activation (Celic et al., 2012; Koulen et al., 2002; Petri et al., 2010; Yang and Ehrlich, 2016). However, these results have not been validated from the ciliary pool of polycystin-2, nor have they provided a mechanistic description of calcium's regulation of channel gating. In this manuscript, we determine which EF hand vertices are most important for calcium affinity. We find that disrupting calcium occupancy of the EF hand does not alter CDM and CDD of mouse and human orthologues of polycystin-2 in the primary cilia. We characterized the kidney phenotype from two new mice that express unique mutations which disrupt Ca²⁺-EF hand association but fail to develop polycystic kidney disease. We measured Gα_{q}-mediated Ca²⁺ release from primary collecting duct cells isolated from WT and mutant mice to test ER localized populations of polycystin-2, but found no difference. Our results suggest Ca²⁺-dependent biophysical regulation of polycystin-2 involves other sites and/or effector proteins, which control channel opening and desensitization. Our findings demonstrate that disrupting of Ca²⁺-EF hand affinity does not lead to impaired in vitro or in vivo function of polycystin-2, which suggests that ADPKD-causing truncating variants found in the CTD likely effect other motifs which have a greater impact on channel regulation.

Results

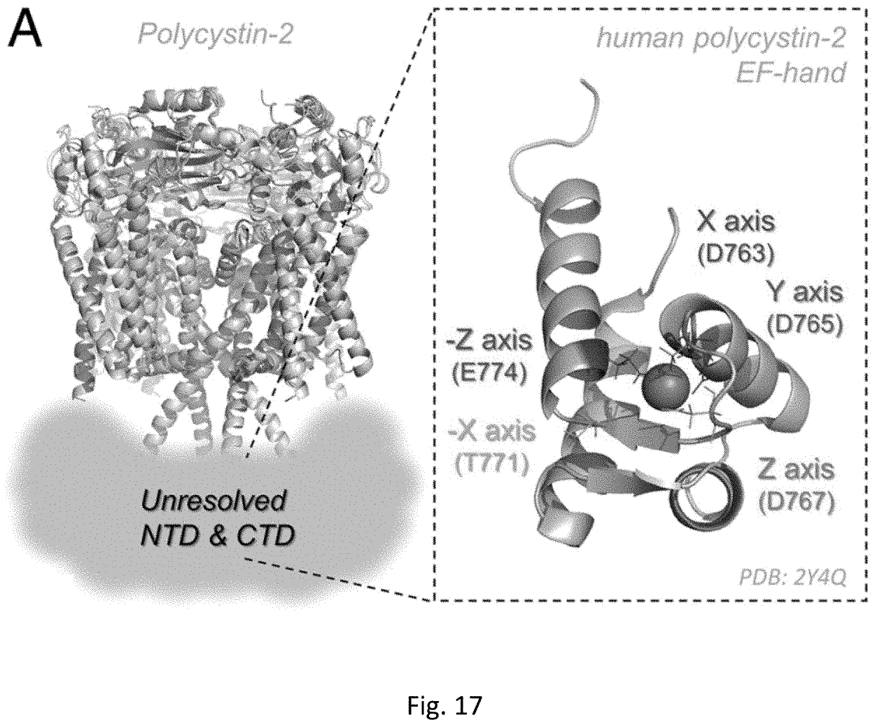

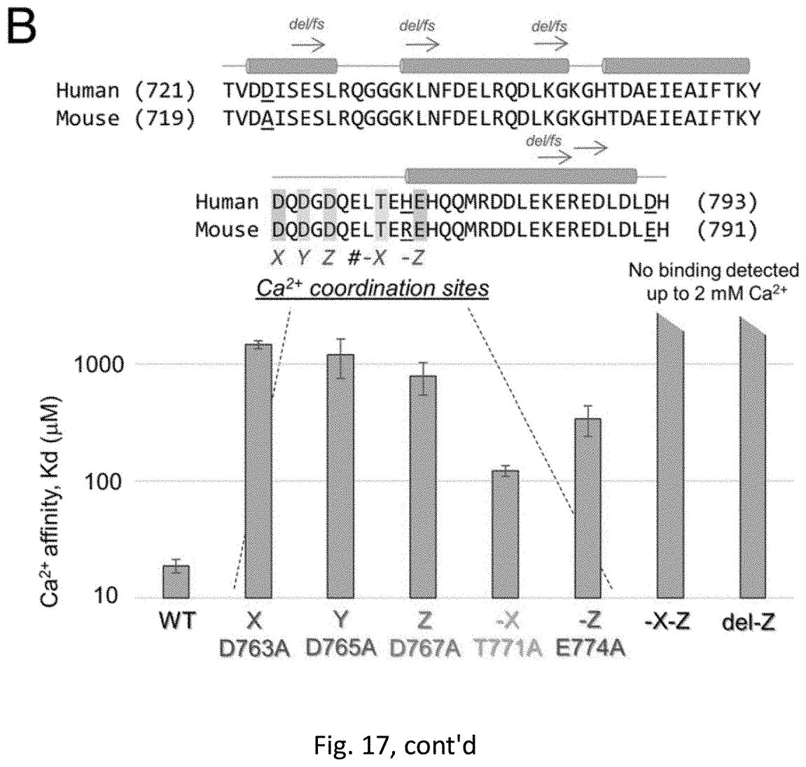

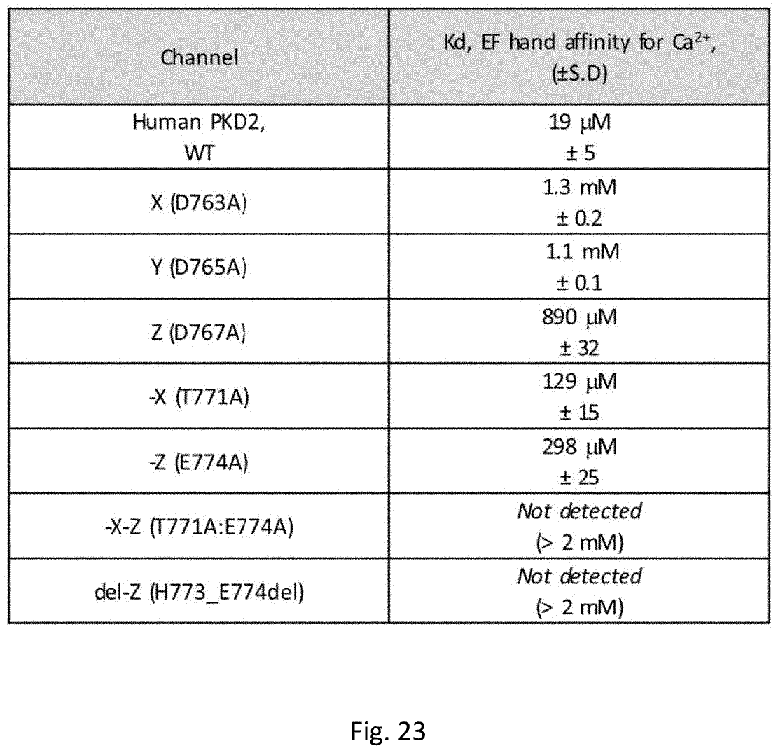

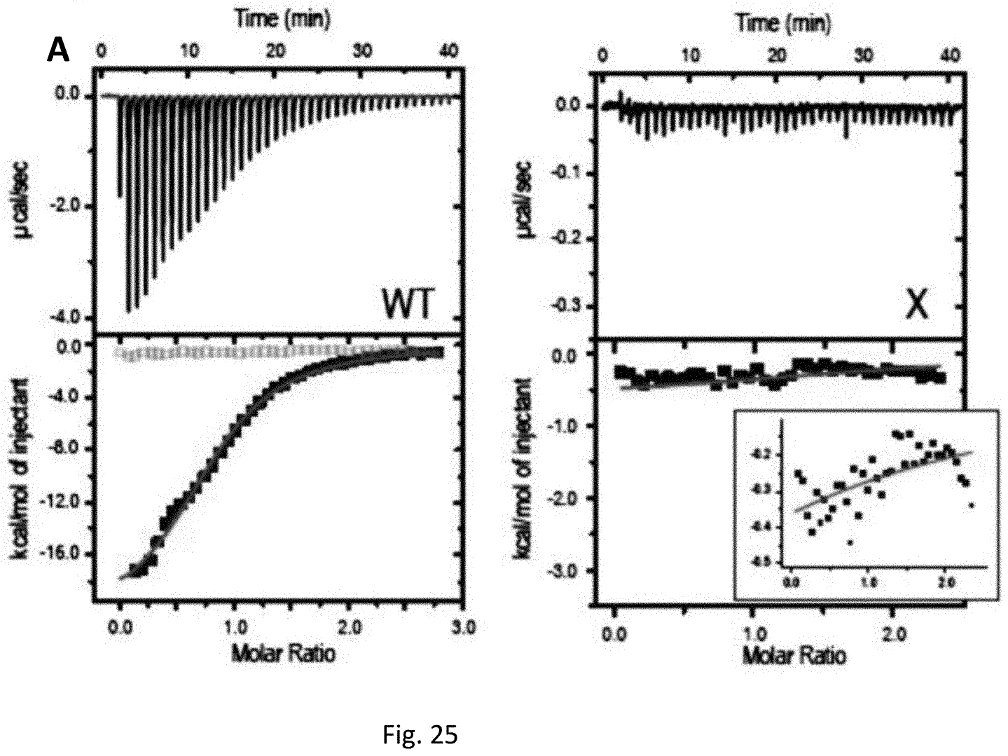

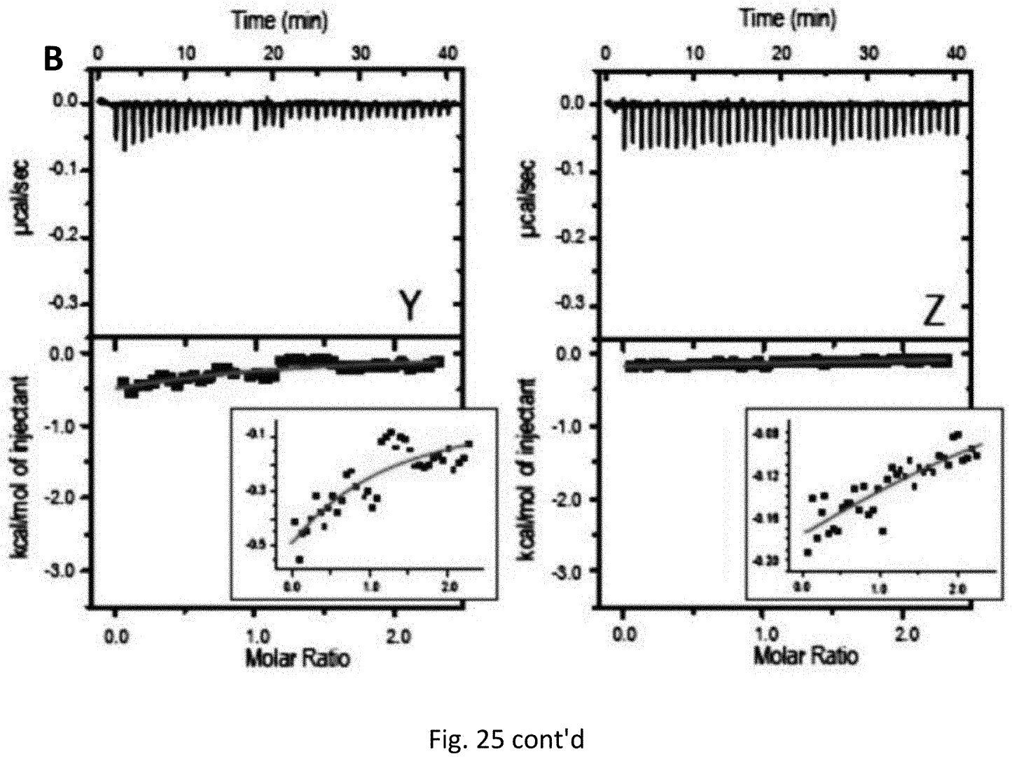

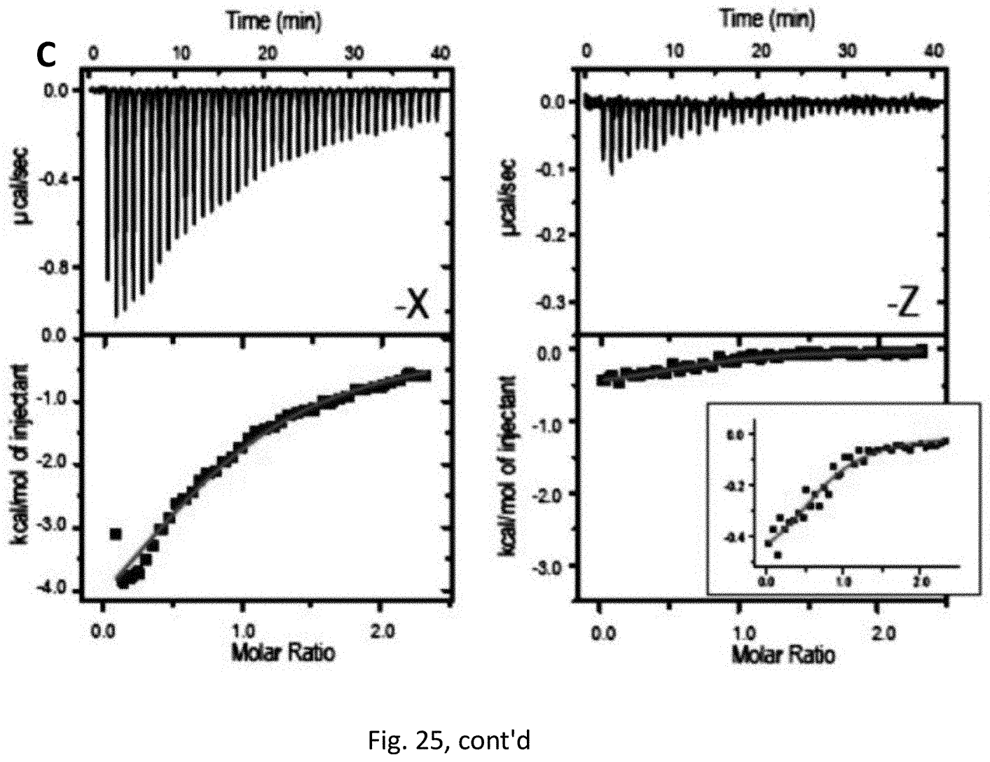

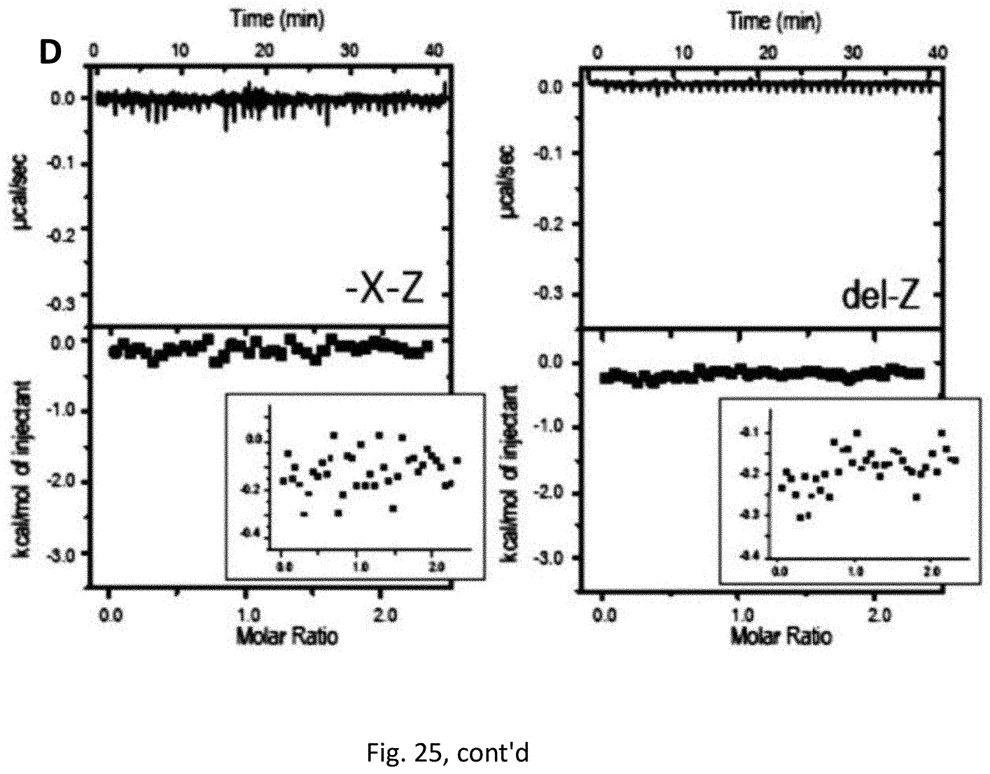

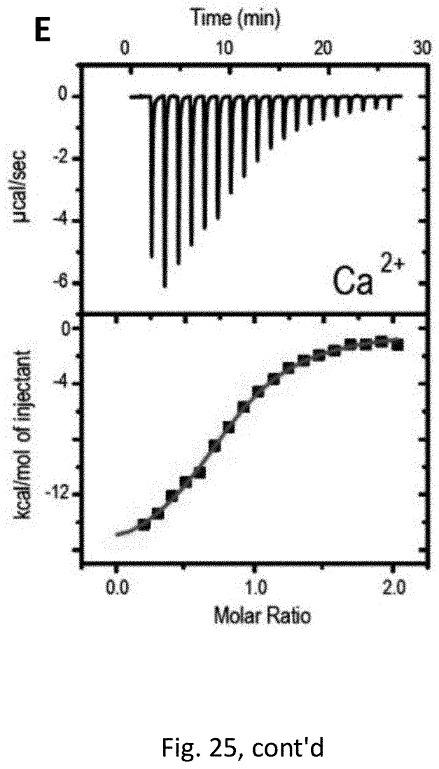

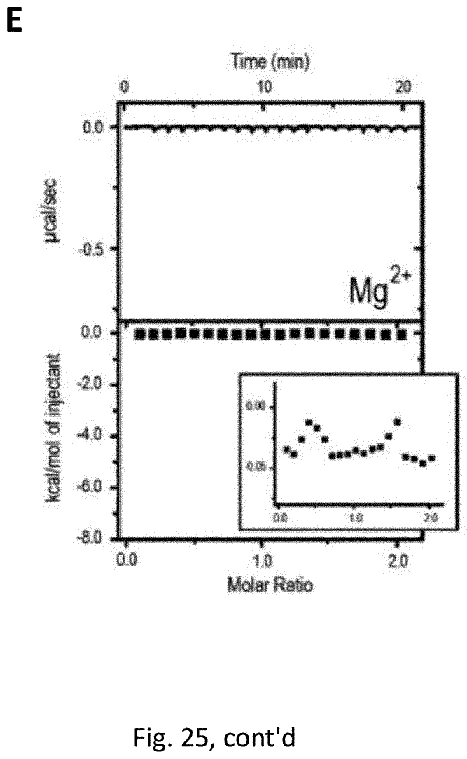

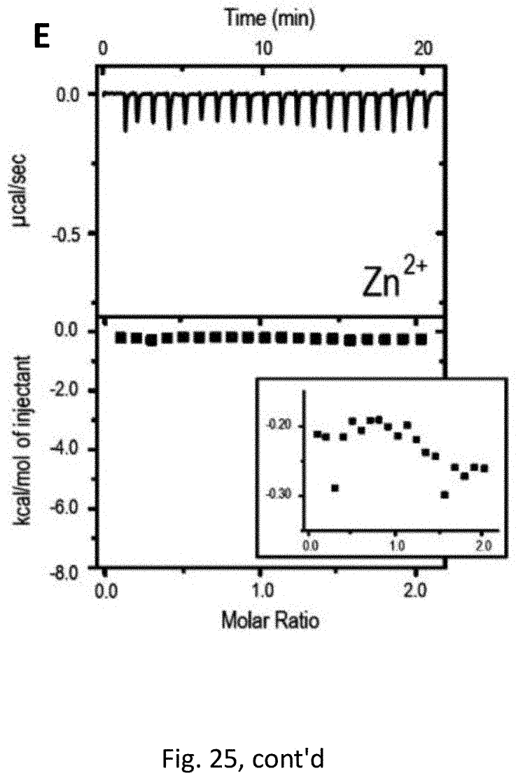

Two groups have independently reported that human and mouse orthologues of polycystin-2 are activated by internal Ca²⁺ using direct cilia electrophysiology (Kleene and Kleene, 2017; Liu et al., 2018). Polycystin-2 channels from both species are highly conserved and contain a C-terminal Ca²⁺-binding EF hand domain-a structural motif that is proposed to confer Ca²⁺ sensitivity in these channels (Celic et al., 2012; Mochizuki et al., 1996; Yang and Ehrlich, 2016). While this motif has escaped structural determination in previously published cryo-EM polycystin-2 structures, isolated EF hand(s) from human and sea urchin orthologues have been determined using crystallographic and NMR methods (Allen et al., 2014; Petri et al., 2010; Yu et al., 2009). The EF hand of human polycystin-2 has five conserved vertices (X, Y, Z, −X and −Z) that coordinate the Ca²⁺ ion with four side chain carboxylate and one backbone carbonyl interactions (FIG. 17A, B). We isolated a CTD peptide of human polycystin-2 (I704-P797), which contains the EF hand and measured changes in heat capacity upon Ca²⁺ addition using isothermal titration calorimetry (FIG. 25A-D). We determined calcium's affinity for the WT human CTD EF hand was 19±5 μM—a finding which approximates the value previously reported (Kd=22 μM) for this peptide (Yang et al., 2015). Importantly, the polycystin-2 EF hand did not have affinity for other physiologically relevant divalent ions, Mg²⁺ and Zn²⁺ (FIG. 25E). We then individually mutated the EF hand vertices to alanine to determine which positions contribute to Ca²⁺ binding. We found that all five vertices are involved in Ca²⁺ affinity for the EF hand, as all alanine substitutions increased Kd between 6-68 times (FIG. 17B, FIG. 23). Based on these results, if Ca²⁺ occupancy of the EF hand regulates polycystin-2 function, disrupting its affinity for this motif should translate into measurable changes in the channel's Ca²⁺-dependent properties.

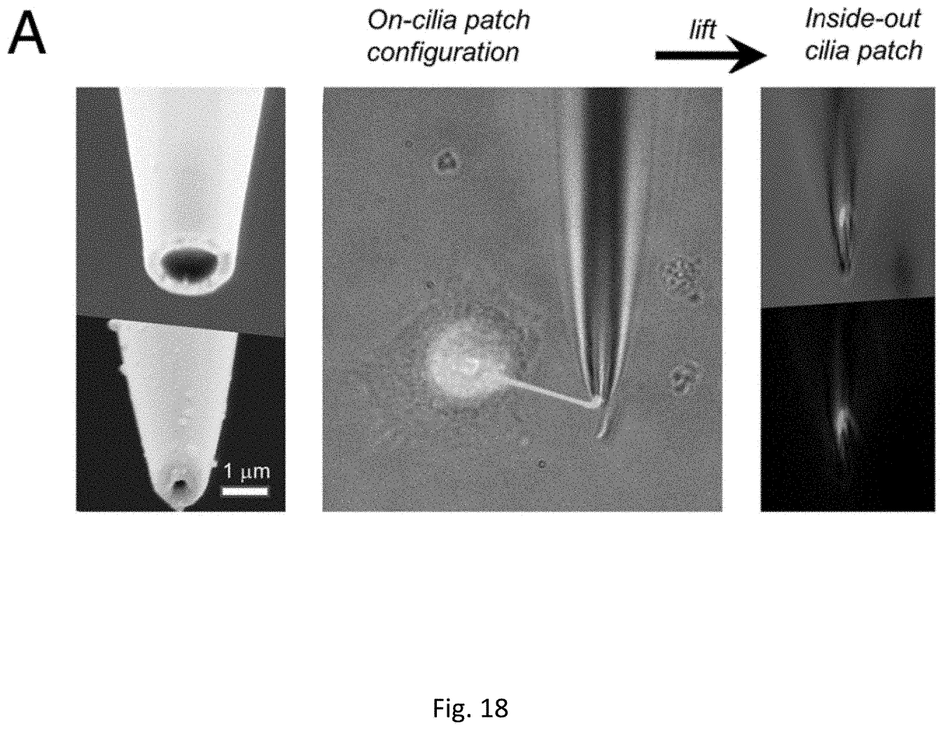

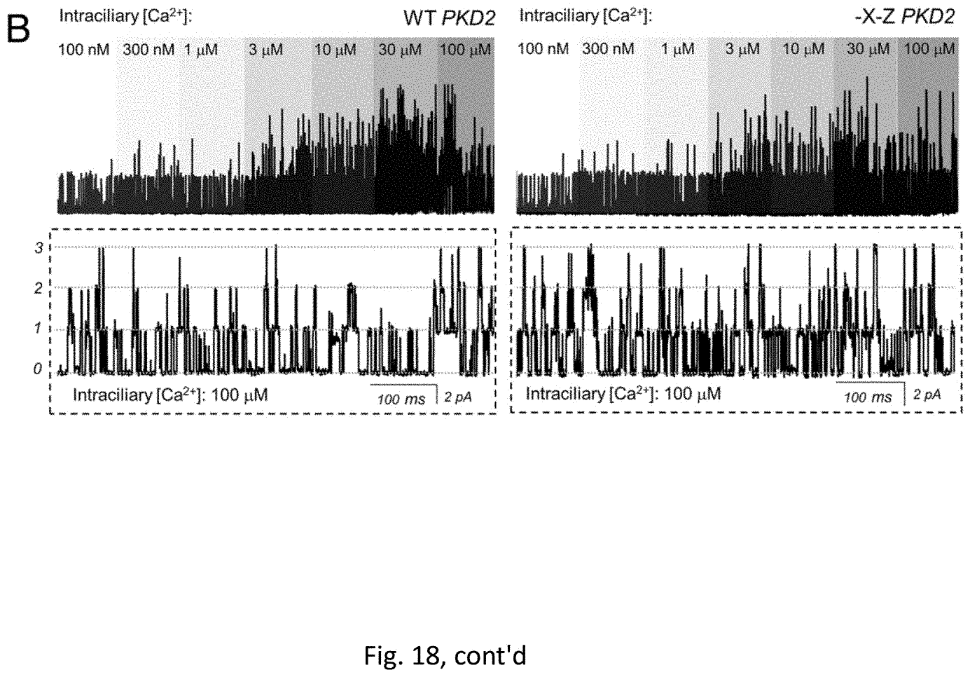

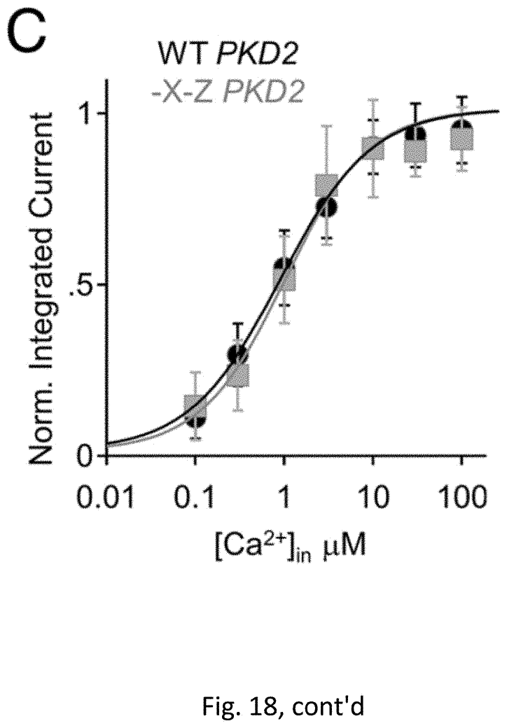

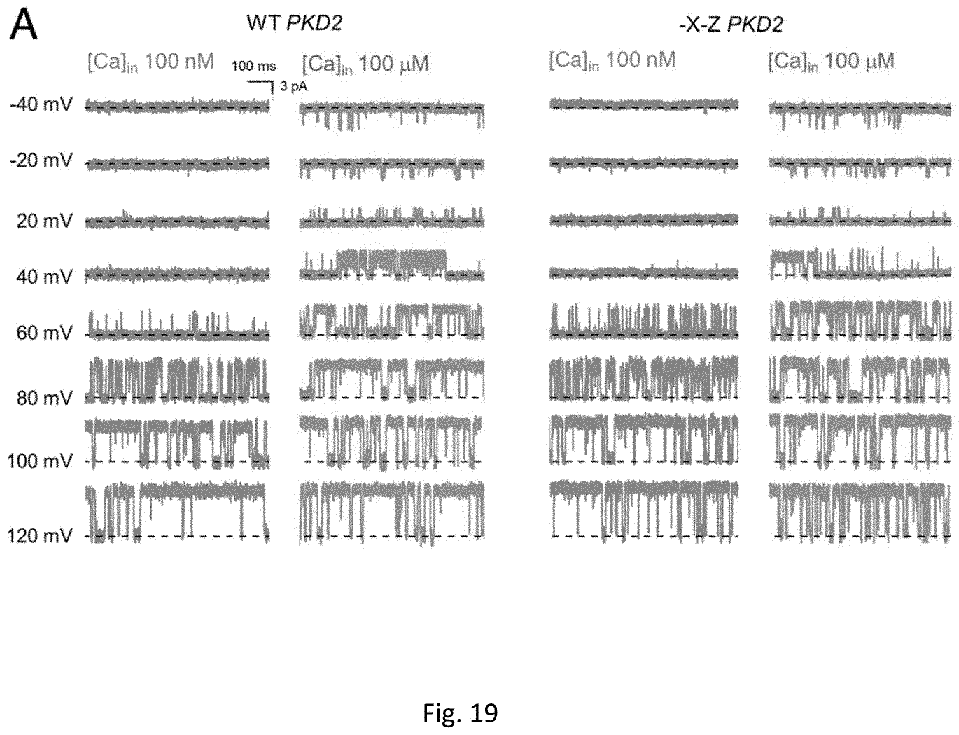

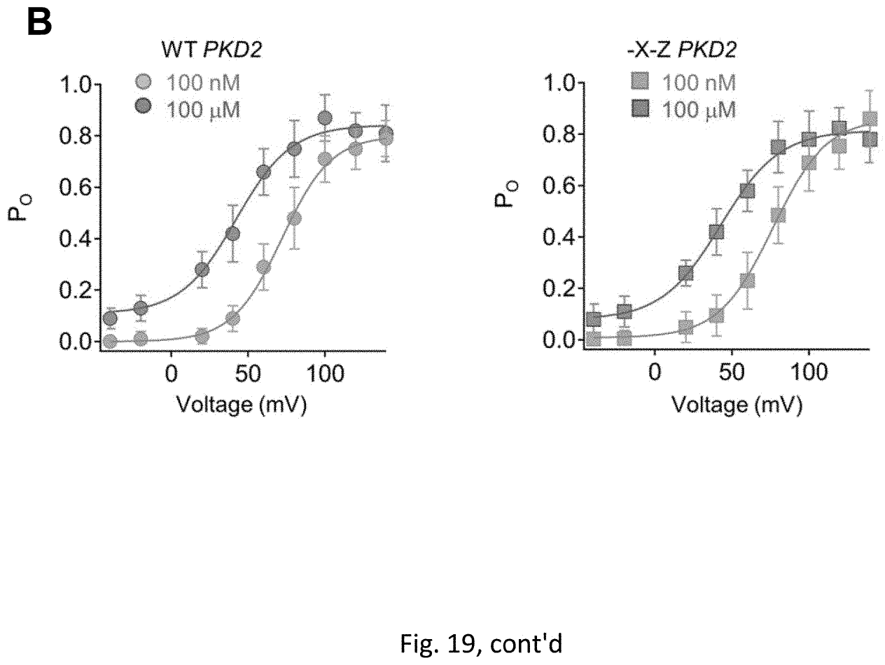

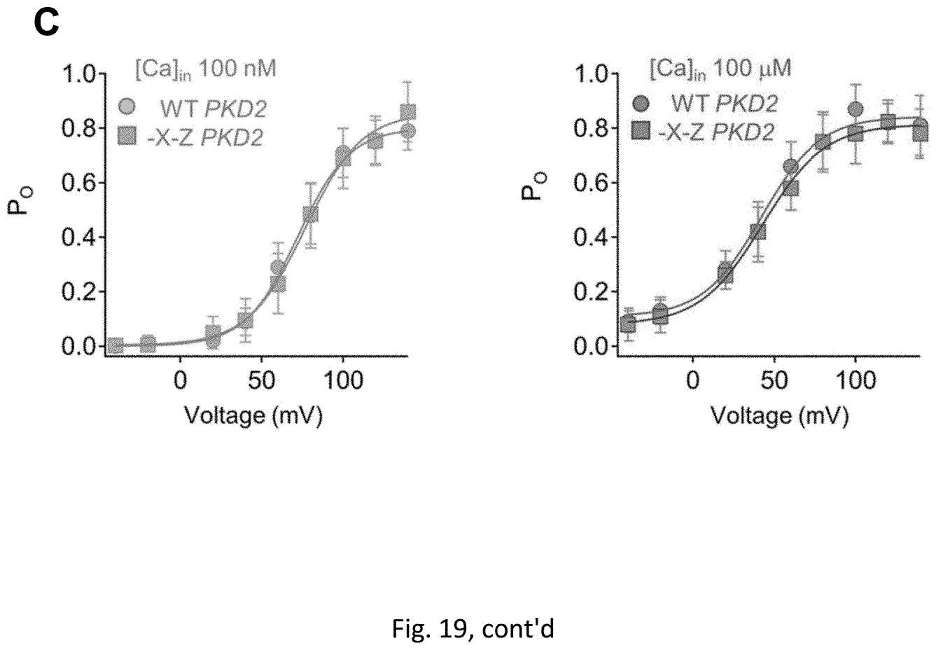

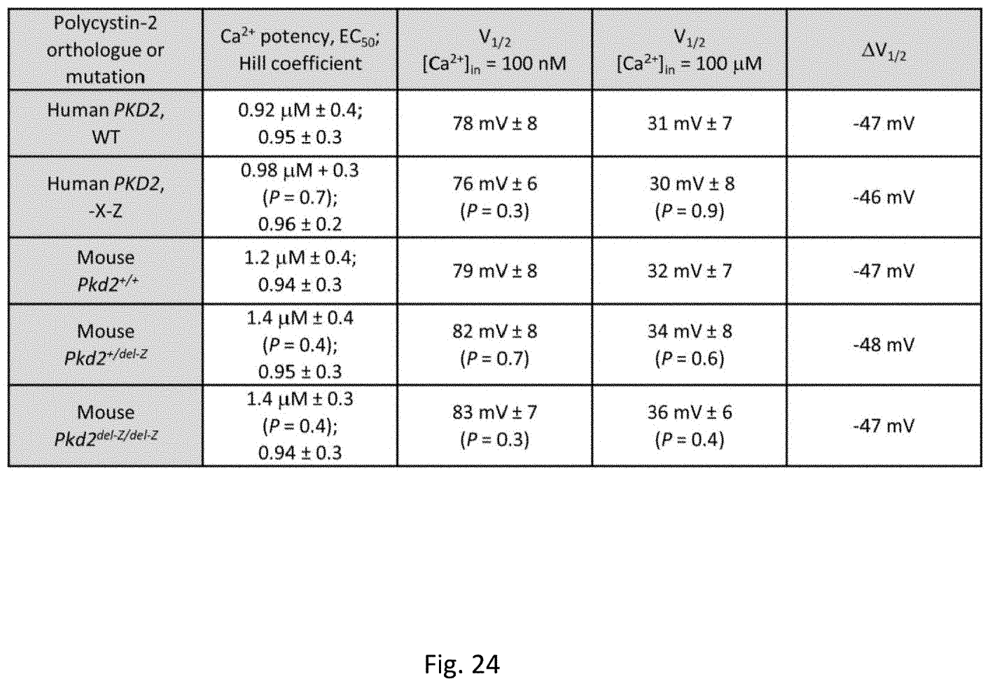

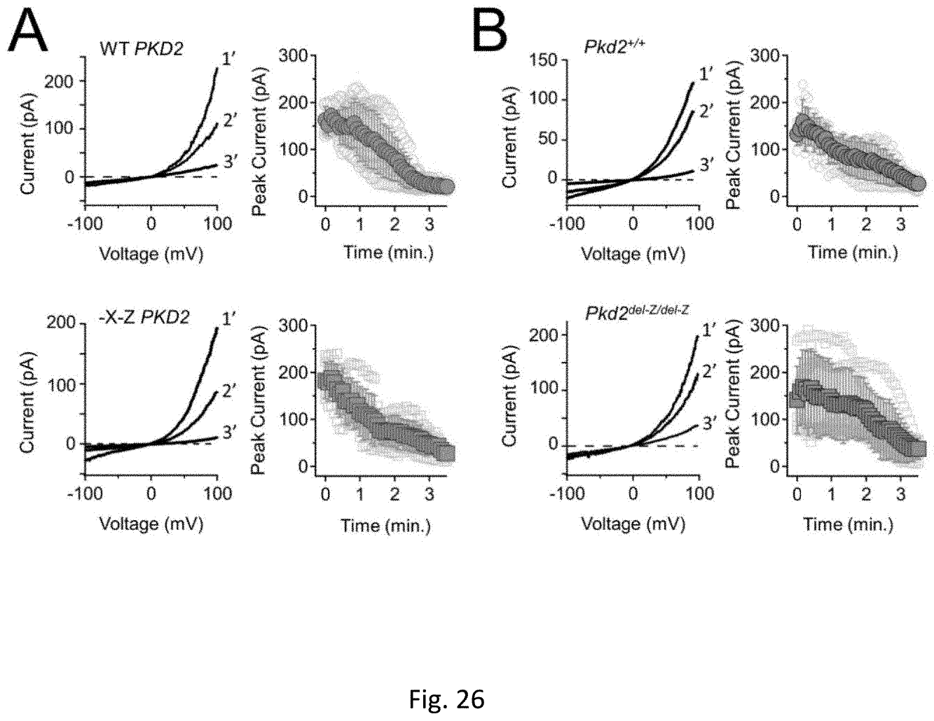

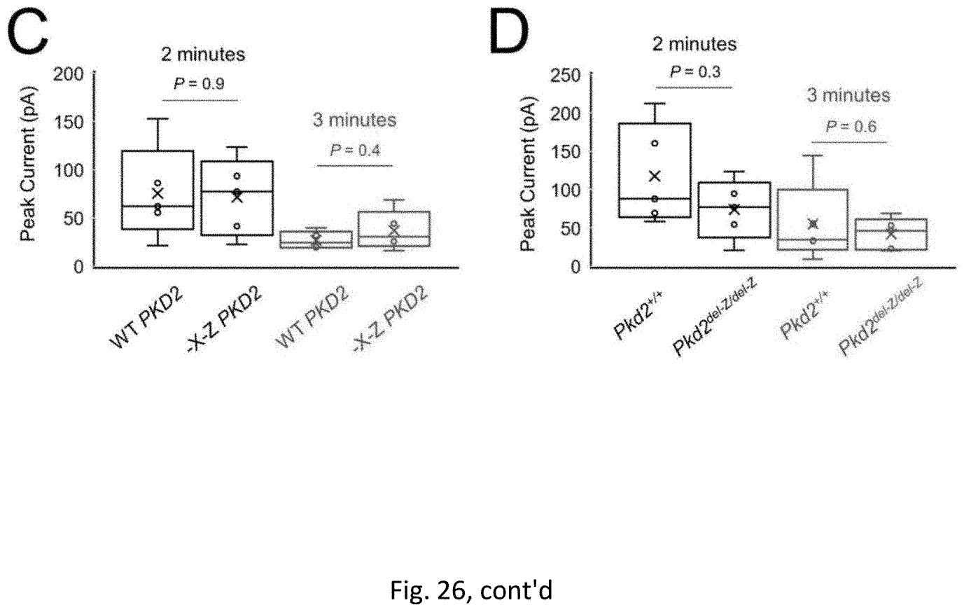

To test whether calcium occupancy of the EF hand is responsible for CDM and CDD of human polycystin-2, we generated double alanine mutations at the −X (T77TA) and −Z vertices (E774A) which abolished Ca²⁺ affinity for the CTD fragment (FIG. 17B). We then expressed the −X-Z double mutation in PKD2-GFP in a PKD2^{null} HEK cell line, so that exogenous channels could be assayed without the contribution of endogenous PKD2 alleles, as previously described (Vien et al., 2020). The stably expressed human WT and −X-Z channels trafficked to the primary cilia, and glass electrodes with submicron apertures were fabricated to form high resistance electrical seals with the GFP-illuminated cilia membranes (FIG. 18A). As previously described, we established inside-out cilia patch configurations so that Ca²⁺ within the cilia (intraciliary) can be adjusted by the superfusate (FIG. 18A, B)(Liu et al., 2018). We observed that the number of single channel events increased when intraciliary Ca²⁺ was elevated from 100 nM to 100 μM for both WT and −X-Z channels (FIG. 18B, C). However, no difference in Ca²⁺ potency (EC50) for opening between WT and mutant channels was observed after fitting the integrated single channel activity to the Hill equation (FIG. 18C, FIG. 24). Previous work has established that CDM is a product of a hypopolarizing-shift in polycystin-2's voltage dependence, which increases channel open probability (Pₒ) at negative membrane potentials (Kleene and Kleene, 2017; Liu et al., 2018). To determine if this mechanism was altered by the −X-Z mutation, we compared voltage dependent channel opening at low (100 nM) and high (100 μM) intraciliary Ca²⁺ concentration (FIG. 19A). These experiments were performed with only one channel present in the membrane, so that stochastic effects on single channel states could be determined. The shift in the voltage-dependence of half maximal open probability (□V_{1/2}) from low to high Ca²⁺ was −47 mV for WT channels, and was not different from the shift observed in −X-Z channels (FIG. 19B, C, FIG. 24). As previously reported, we observed that polycystin-2 enters an irreversible desensitized state (CDD) after prolonged exposure to high internal calcium (Liu et al., 2018). To test the EF hand role in this form of channel regulation, we conducted whole-cilia recordings using 30 μM intraciliary free calcium and plotted the time course of current desensitization (FIG. 26A, C). The time course of CDD was not different between WT and −X-Z mutant channels, as the magnitude of cilia current is completely abolished for both channels after 3.5 minutes of recording. Taken together, these results demonstrate that abolishing Ca²⁺ occupancy of the human polycystin-2 EF hand does not alter CDM and CDD forms of channel regulation.

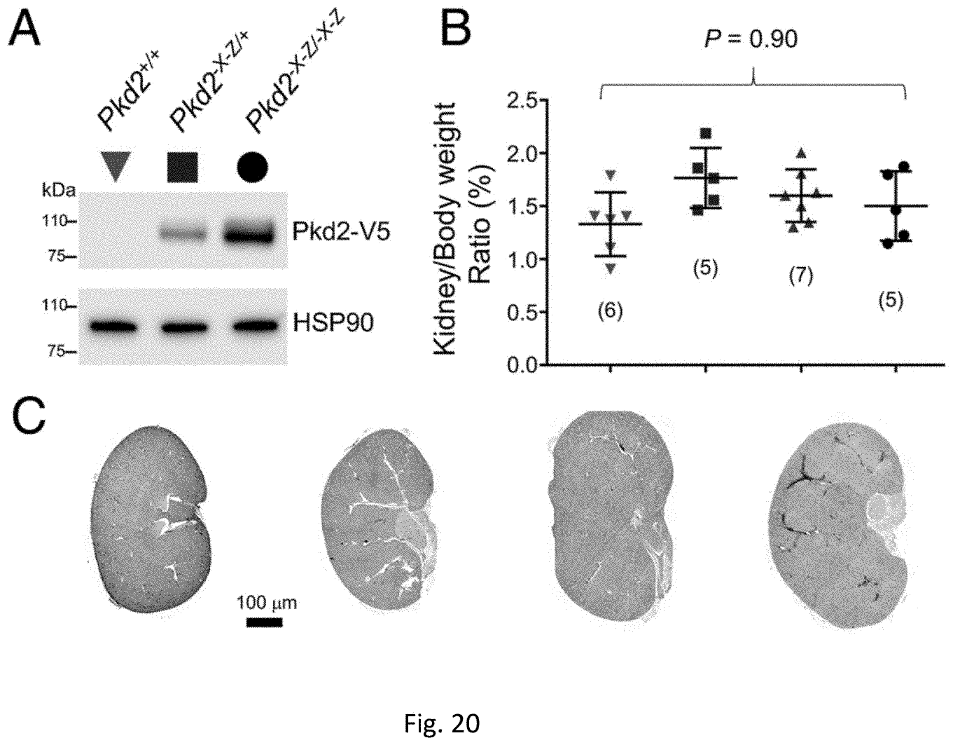

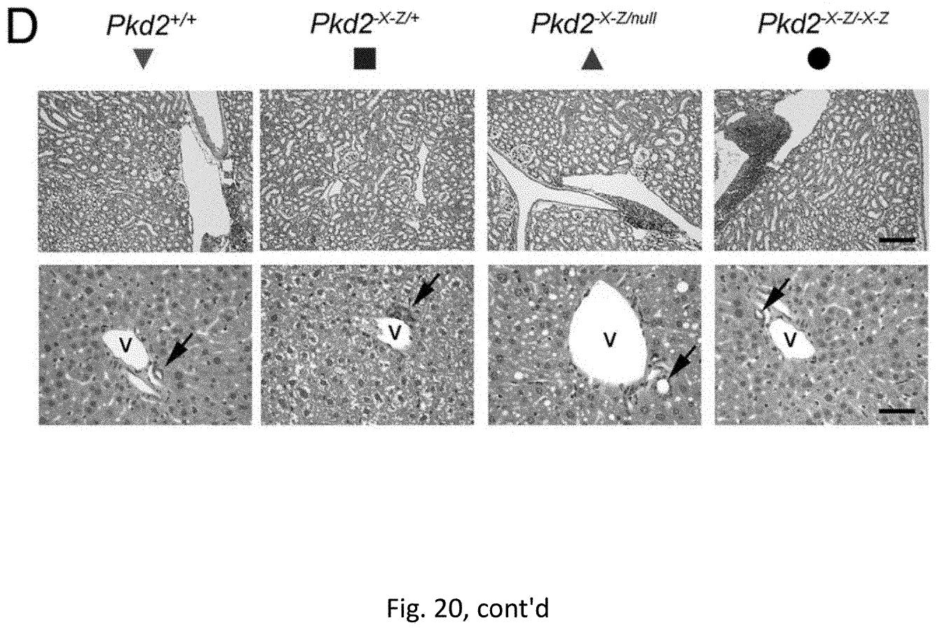

Previous work disrupting polycystin-2 function by either allelic ablation or truncation of Pkd2 resulted in embryonic lethality for homozygous mice (Wu et al., 1998; Wu et al., 2000). Conditional and kidney specific ablation of Pkd2 results in penetrant and reproducible polycystic kidney phenotype in mice (Ma et al., 2013). Since the −X-Z mutation had no impact on polycystin-2 function, we hypothesized that homozygous animals expressing analogous mutations would likely survive and the allelic impact of abolishing Ca²⁺ binding on cystogenesis in the kidney could be assessed in vivo. Furthermore, since the most sensitive bioassay for loss of polycystin function is cyst formation, we sought to determine whether the normal channel function without EF hand calcium affinity is consistent with normal in vivo function by generating the equivalent −X-Z knock-in mouse model. To make the Pkd2^{-X-Z} mouse, we employed the CRISPR/Cas9 method to substitute the −X (T769A) and −Z (E772A) vertices and simultaneously insert a V5 epitope tag immediately before the termination codon in murine Pkd2 (Tran et al., 2019). We confirmed expression of the mutated epitope tagged protein in the kidney lysates by immunoblot analysis with an anti-V5 antibody (FIG. 20A). The animals were viable without developing either kidney or liver cysts up to 18 months (data not shown). Mice were systematically examined at 9 months of age showing normal kidney weight (KW) and liver weight (LW) as a fraction of body weight (BW) and normal histological appearance without evidence of kidney tubular bile duct dilation or cyst formation (FIG. 20B-D). Pkd2^{+/-} mice were crossed with Pkd2^{-X-Z/-X-Z} animals to generate Pkd2^{-X-Z/Null} mice to examine whether the reduced dosage can elicit a phenotype. These mice also did not display kidney or liver cysts at 9 months of age. The aggregate biophysical and in vivo results demonstrate that abolishing Ca²⁺-EF hand affinity using −X-Z vertices neutralizing mutations does not alter polycystin-2 ion channel function, nor does it engender sufficient loss of function to cause polycystic kidney disease in mice.

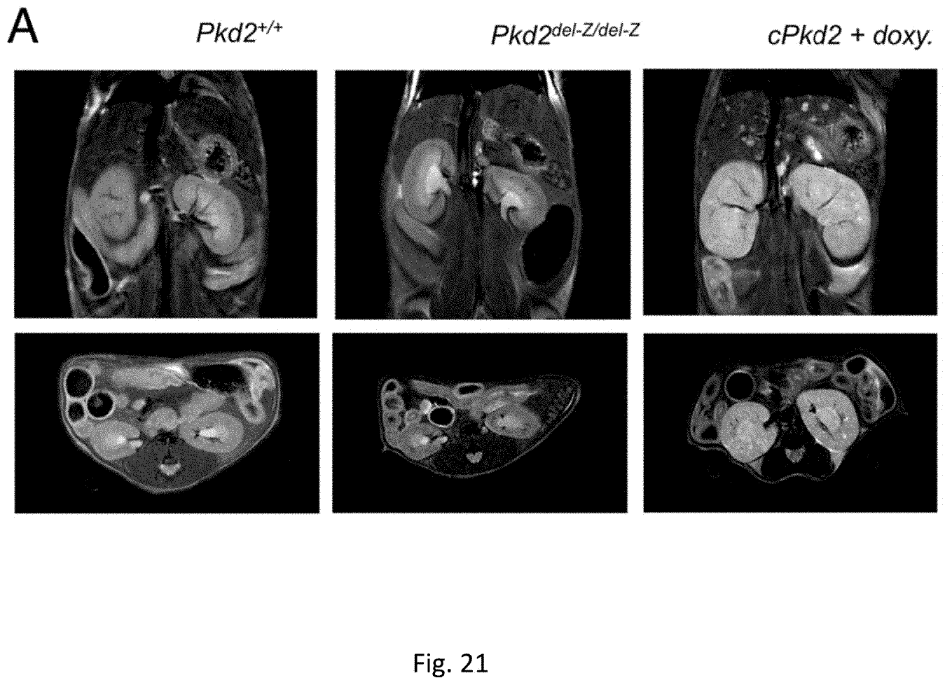

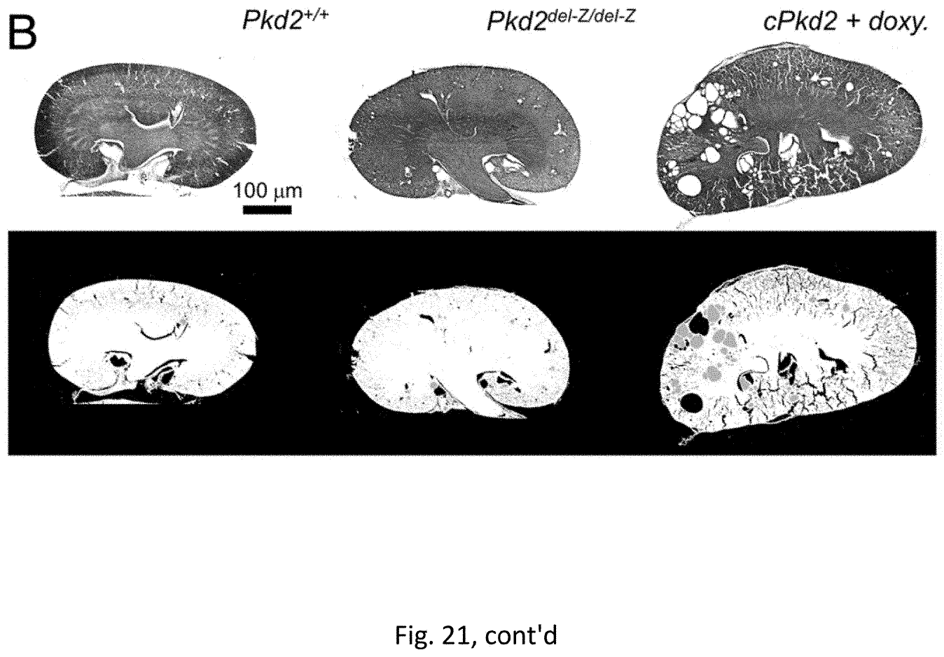

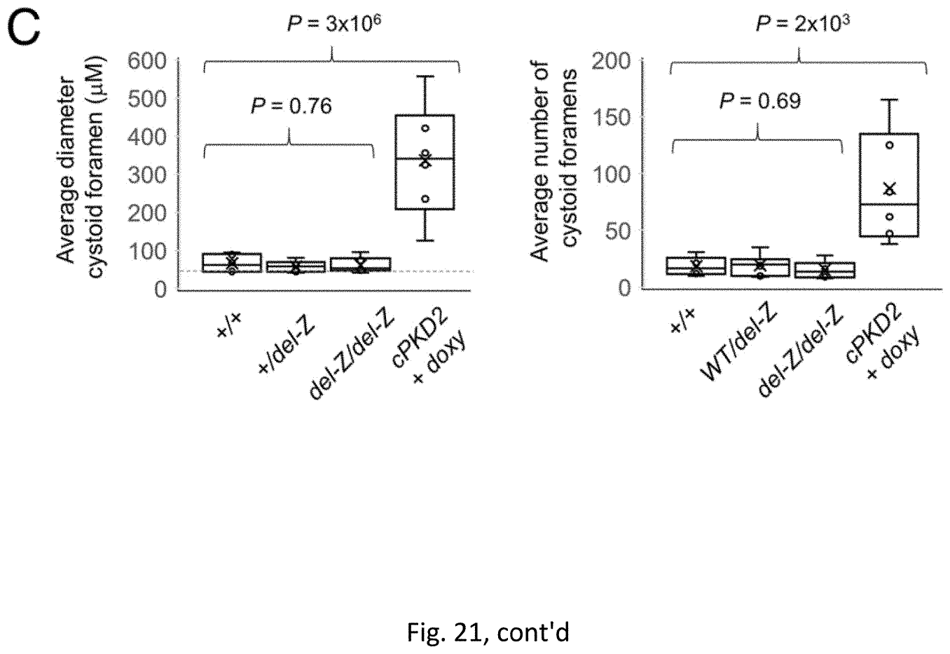

This result excludes the hypothesis that Ca²⁺ occupancy of the EF hand is necessary for polycystin-2 function, and when disrupted, contributes to PKD. Thus, we challenged the robustness of this interpretation by generating a second mouse strain (Pkd2^{del-Z}), in which the EF hand −Z vertex (E772), along with the preceding arginine (R771) were genetically deleted. Using isothermal titration calorimetry measurements, we confirmed that del-Z mutation abolished calcium occupancy of the EF hand (FIG. 17B, FIG. 25E). Pkd2^{+/del-Z} Pkd2^{del-Z/del-Z} and Pkd2^{del-Z/-} mice were viable to at least 12 months of age, and the kidney morphology from these mice were periodically imaged using MRI (FIG. 21A). As a positive control, we imaged the development of PKD cysts from cPkd2 mice, where expression of Pkd2 was attenuated by doxycycline induction after 6 months of life (Ma et al., 2013). Development of PKD-type kidney cysts was apparent in all (10/10) of the induced cPkd2 mice after one year, whereas none of the Pkd2^{+/+} (0/18), Pkd2^{+/del-Z} (0/19), and Pkd2^{del-Z/del-Z} (0/18) animals developed PKD-type cysts. In our preliminary observations, a minority of the Pkd2^{del-Z/del-Z} (4/35) mice developed bilateral cystic disease (non-PKD-type), but because of the infrequency of this phenotype and because these cysts were not observed after outbreeding, we conclude that this bilateral cyst phenotype is unlikely caused by the del-Z mutation. Due to the limited resolution of MRI, we conducted histology experiments on sectioned kidneys from six mice from each genotype. Consistent with the MRI results, we did not observe any obvious cysts in the kidney sections from the other mice expressing homozygous for Pkd2^{del-Z} alleles (FIG. 21B). To quantify this observation, we conducted image analysis of the average number and size of cystoid foramen in the sections (FIG. 21C). Here, all cPkd2 mice had significantly more and larger cystoids, whereas the sections isolated from mice expressing one or two copies of the Pkd2del-z allele were not different from WT animals. Our results generated from two mouse strains expressing unique mutations demonstrate that abolishing calcium affinity for the EF hand does not produce polycystic kidney disease in vivo.

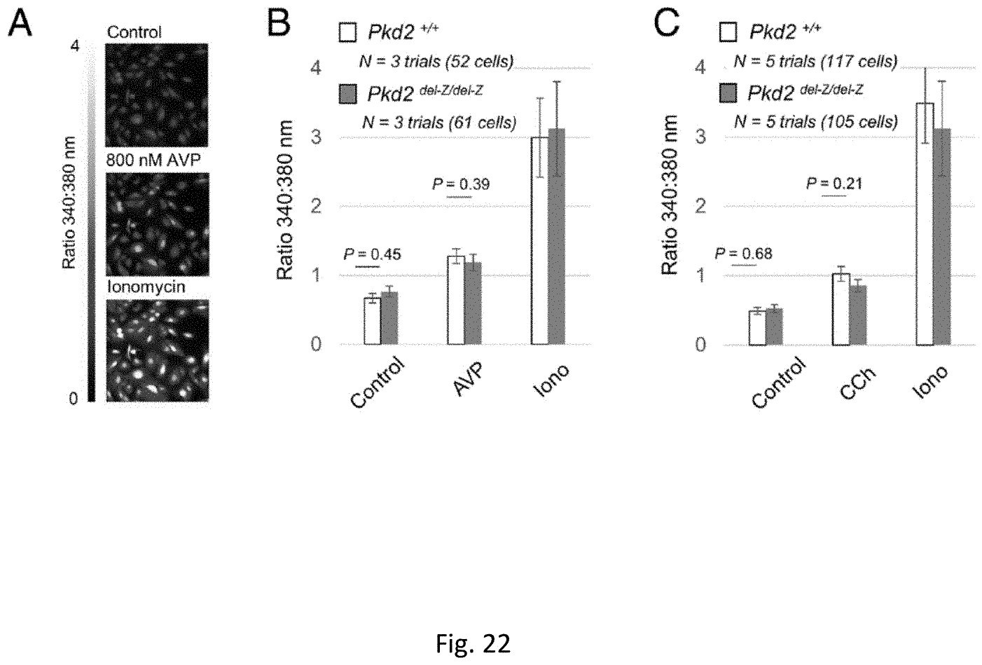

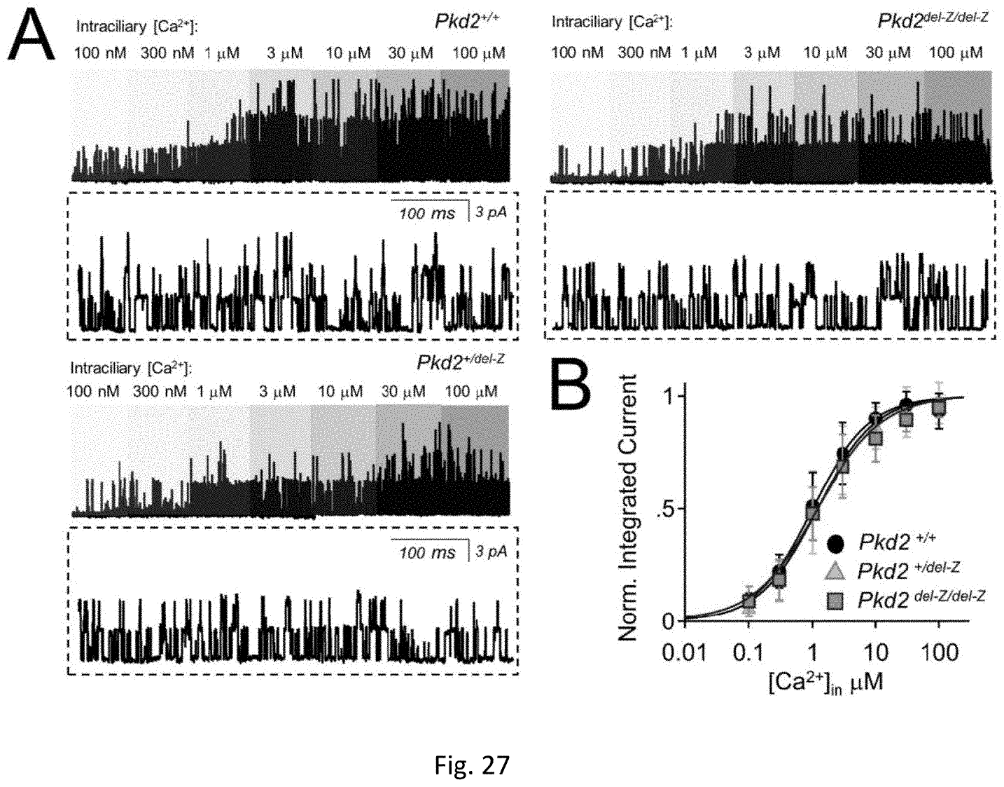

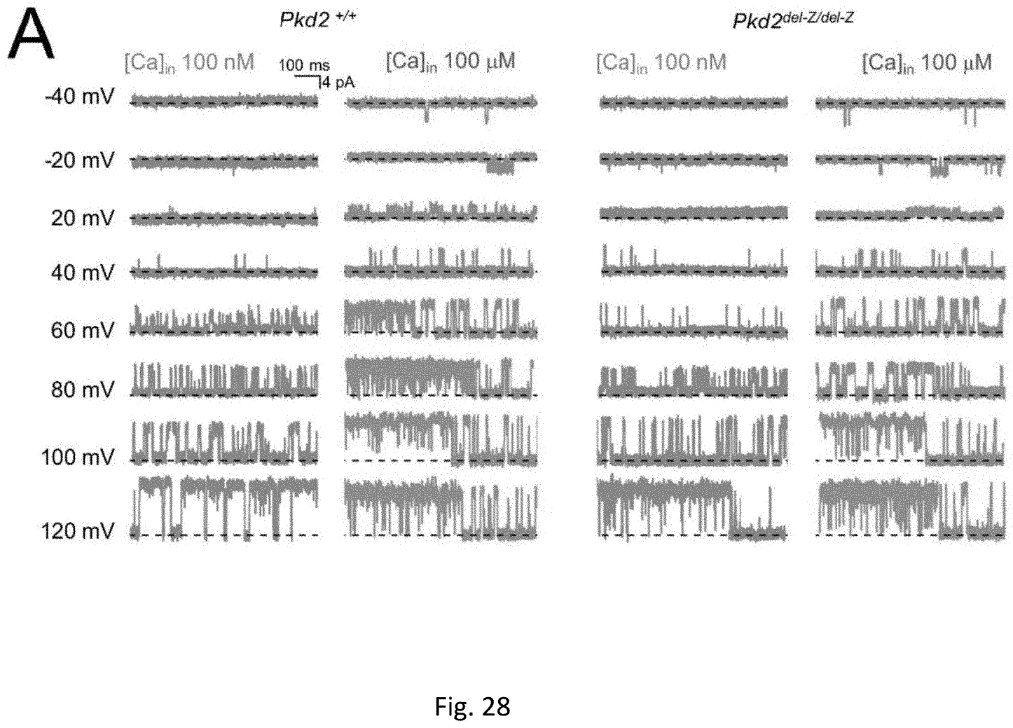

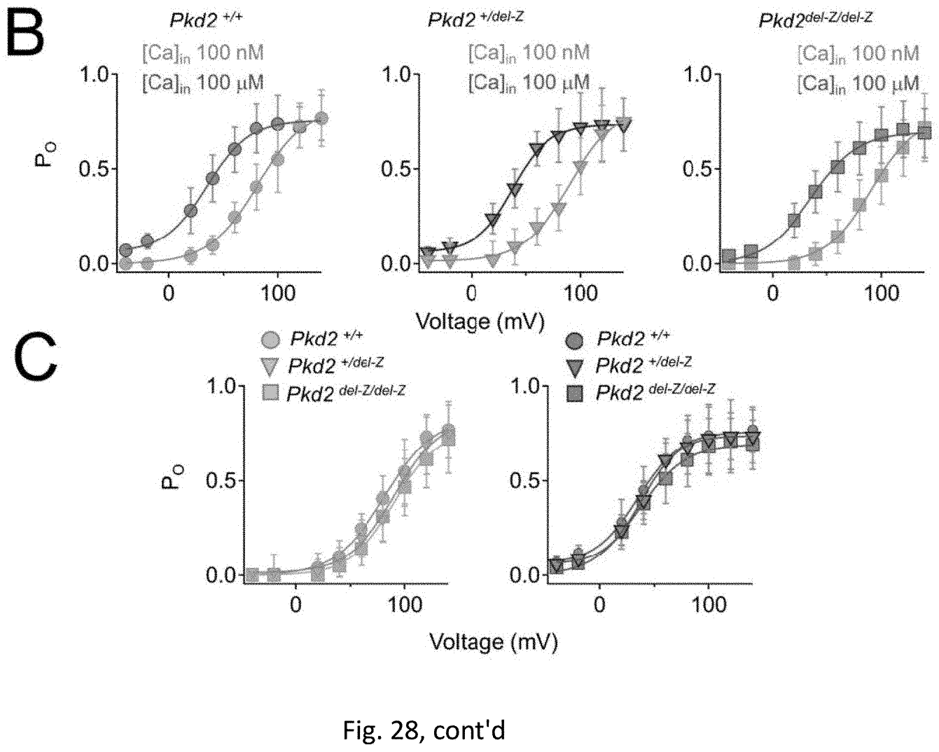

In our previous experiments, we established that disrupting Ca²⁺-EF hand affinity does not alter the biophysical properties of the human orthologue of polycystin-2 under heterologous expression. To test these effects on the mouse channel orthologue under endogenous expression in the kidney collecting duct, we crossed the Pkd2^{del-Z} mice with our cilia specific reporter ARL13B-EGFP strain, where all primary cilia become fluorescent under GFP excitation (DeCaen et al., 2013; Liu et al., 2018). We then isolated collecting duct cells (pIMCD) from Pkd2^{+/+}, Pkd2^{+/del-Z}, Pkd2^{del-Z/del-Z} mice and compared CDM of polycystin-2 using the inside-out patch clamp configuration. As expected, the voltage dependence of single channel opening for all three genotypes proportionally shifted to negative membrane potentials when internal Ca²⁺ was elevated-demonstrating that the CDM mechanism is still functional, a result shared by our human channel results (FIG. 27). The half-maximal voltage-dependence of channel opening (V_{1/2}) was not different from the three genotypes at low calcium. Under elevated intraciliary calcium, we observed a small but insignificant difference (−4 mV, P=0.4) in V_{1/2} when comparing the cilia channels measured from Pkd2^{+/+} and Pkd2^{del-Z/del-Z} mice (FIG. 24). The potency of calcium opening polycystin-2 channels was not different from Pkd2^{+/+} (EC₅₀=1.2±0.4 μM), Pkd2^{+/del-Z} (EC₅₀=1.4±0.4 μM), and Pkd2^{del-Z/delZ} (EC₅₀=1.4±0.3 μM) mice (FIG. 28, FIG. 24). In addition, we observed no difference in the onset of CDD between cilia currents recorded from Pkd2^{+/+} and Pkd2^{del-Z/del-Z} mice (FIG. 26B, D). As discussed previously, polycystin-2 was reported to function as an intracellular Ca²⁺ release channel (Koulen et al., 2002). Here, vasopressin stimulated Ca²⁺ release was attenuated by heterologous over-expression of a truncating mutation (L703X) in a porcine kidney-derived cell line (LLC-PK1). Since L703X removes the EF hand along with the entire CTD in polycystin-2, it is possible that removing the EF hand motif was responsible for the attenuated calcium response to vasopressin (Koulen et al., 2002). To determine if Ca²⁺-EF hand occupancy might affect the function of the ER localized population of polycystin-2, we compared Gα_{q}-mediated Ca²⁺ store release from primary collecting duct cells isolated from Pkd2^{+/+} and Pkd2^{del-Z/del-Z} mice. (FIG. 22). However, vasopressin and carbachol stimulated Ca²⁺ store release was indistinguishable between these genotypes, despite removing calcium's affinity for the EF hand using the del-Z mutation. Taken together our results demonstrate that murine polycystin-2, in the ER and primary cilia membranes, are nominally impacted by disrupting Ca²⁺-EF hand affinity and does not engender loss of function in vivo as it relates to ADPKD.

Discussion

Many ion channels are regulated by intracellular Ca²⁺—a feature which is tied to their physiological regulation in cell membranes. Like polycystin-2, the conductive properties of voltage-gated sodium (Naᵥs) and calcium channels (Caᵥs) and calcium-activated potassium channels (K_{Ca}) are modulated by internal Ca²⁺ (Xia et al., 2002) (Chagot et al., 2009; Guo et al., 2016; Nejatbakhsh and Feng, 2011). Interestingly, all members of these channel families have C-terminal EF hands. However, there are clear differences in the involvement of this motif in their regulation by intracellular calcium. An example of a channel regulated by EF hand-Ca²⁺ association is the cardiac sodium channel, Naᵥ1.5 (Wingo et al., 2004). Here, several missense variants associated with arrhythmia syndromes localize to the EF hand, which causes structural misfolding of the CTD and shifts Nay1.5 steady-state inactivation (Gardill et al., 2018). However, the EF hand is not solely responsible for Ca²⁺ regulation in these channels. Rather, the structural components of the Naᵥ-Ca²⁺-sensing apparatus also includes an ‘inactivation gate’ formed by their inter-domain loop motif and their CTD association with calmodulin—a ubiquitous, EF hand containing Ca²⁺ sensor protein (Johnson et al., 2018; Shah et al., 2006; West et al., 1992). Conversely, inactivation of P/Q and L-type Cavs are structurally regulated their CTDs but mutations which disrupt EF hand-Ca²⁺ affinity but do not alter their function (Zhou et al., 1997), leading researchers to explore other potential sources of the Ca²⁺-sensing mechanism (DeMaria et al., 2001; Lee et al., 1999). Based on our findings, Ca²⁺ regulation of polycystin-2 appears to be fall on later example, as our results clearly demonstrate that Ca²⁺-EF hand association is not required for CDM or CDD, nor is it required for in vivo channel function as it pertains to ADPKD. How then does polycystin-2 sense Ca² and in response alter its gating? We hypothesize that Ca²⁺ is acting on undetermined receptor site(s) within the channel, or that an unknown Ca²⁺-binding protein associates with polycystin-2 to regulate its gating. Results from calorimetry and NMR spectra methods suggest that the EF hand is the only Ca²⁺ binding site in the CTD of polycystin-2 (Yang et al., 2015). However, it is important to note that the complete channel protein were not tested in these studies and in our data set, which leaves possibility that the remaining portion of the channel may contain additional Ca²⁺ coordinating sites. Since the CDM can be observed in the inside-out patch configuration, our results suggest that Ca²⁺ is either acting directly, or involves an interacting ‘Ca²⁺-sensor’ protein which is not readily disassociated from the channel-such as a pre-associated effector protein or membrane embedded factor. This feature is observed in Naᵥ, Caᵥ and in the small (SK) and large-conductance Ca²⁺-activated K⁺ (BK) channels, where separate Ca²⁺-sensor proteins bind to motifs within the channel to control gating (Ben-Johny and Yue, 2014; Fanger et al., 1999; Lee and MacKinnon, 2018; Wang et al., 2014). Polycystin-2 interacts with a number of different proteins, however it is unknown if their association is Ca²⁺-dependent (Morick et al., 2013; Sammels et al., 2010; Streets et al., 2010). Recently, calmodulin was reported to bind and regulate the function of polycystin-2L1-but this relationship is undetermined for polycystin-2 regulation by any other Ca²⁺ effector proteins (Park et al., 2019). It is not clear if there is one or perhaps multiple sites that regulate CDM and CDD in either polycystin. What is clear, is that Ca²⁺ affinity for the EF hand is dispensable for the function of polycystin-2 and polycystin-2L1 channels (DeCaen et al., 2016). Furthermore, selectively neutralizing the EF hand vertices responsible for calcium affinity does not result in cystic kidney disease in mice. CDM and CDD are critical forms of channel regulation, which respectively turns ‘on’ and ‘off’ the flow of Ca²⁺ within the cilia and from the ER. Thus, future work elucidating the components and structural elements responsible will be critical for understanding polycystin-2 molecular regulation, which is demonstrably involved in ciliary function and initiation of cystogenesis in ADPKD.

We have reported that the EF hand of polycystin-2 coordinates Ca²⁺ with low affinity (Kd=19 μM), in agreement with previous calorimetry studies (Celic et al., 2008; Yang and Ehrlich, 2016). This relatively weak affinity would seem irrelevant to ion channels of the plasma membrane, where free calcium (˜90 nM) is regulated by cytoplasmic buffering proteins. However, since the primary cilia retains a high concentration of Ca²⁺ (390-580 nM) at rest and the N- and CTD intracellular domains are near the ion conducting pore, they are likely to experience elevated calcium in the micromolar range (Delling et al., 2013). The CTD contains coiled-coil and EF hand motifs, which have structurally solved separately as fragments using crystallographic and nuclear magnetic resonance methods (Allen et al., 2014; Petri et al., 2010; Zhu et al., 2011). Previous work measuring Ca²⁺ release from ER localized polycystin-2 have determined that CTD truncating mutations which also remove the EF hand cause a complete loss of channel gating, possibility due to loss of calcium-channel affinity or lack of channel subunit oligomerization (Yang and Ehrlich, 2016; Yang et al., 2015). Although our work demonstrates that abolishing Ca²⁺-EF hand occupancy of polycystin-2 does not alter Ca²⁺-dependent regulation, the CTD in its entirety may still be involved in allosterically regulating the channel pore or its assembly. Based on the low resolution density maps used to solve the core structures of polycystin-2, the N- and C-terminal ends of the polycystin peptide form a multimeric structure on the internal side of the channel (Grieben et al., 2017; Shen et al., 2016; Wilkes et al., 2017). Although the contiguous CTD is not resolved either in the polycystin-2 homomeric channel structure or in its complex with polycystin-1 (Su et al., 2018), this site might be involved with controlling the opening of the lower gate through its interactions with the extended intracellular portion of the S6 helix. Our in vivo results demonstrate that two independent mouse models that harbor unique EF hand mutation types which abolish Ca²⁺ affinity-do not manifest the polycystic kidney disease phenotype. Among ˜6000 ADPKD-associated sequences analyzed, there are no reported in-frame variants (missense, insertion and deletions) in the coding region of the PKD2 EF hand (https://pkdb.mayo.edu/, PCH personal communication). However, there are several reported frameshift variants, which result in premature truncations of the CTD (FIG. 17B). These clinical observations in conjunction with our findings suggest that mutations which remove portions of the CTD will have greater impacts on polycystin-2 gating and result in PKD-type cystogenesis, while those that alter calcium occupancy of the EF hand are likely benign.

Methods

CTD protein expression, purification, and isothermal titration calorimetry. The C-terminal fragment of the human polycystin-2 (I704-P797) was cloned into the PET19b vector and transformed into BL21 (DE3) competent cells (New England Biolabs) for bacterial expression. The translated peptide has a 10×His tag on its N-terminus. The alanine mutants of the individual calcium-coordinating residues were created and transformed as well. Cells were grown in 2XYT broth in a 37° C. shaking incubator until OD600 reaches ˜0.6, and were induced by 0.4 mM isopropyl 1-thio-β-D-galactopyranoside and grown for another 4 hours. Cells were harvested and resuspended in Buffer A (500 mM NaCl, 20 mM Tris/HCl, pH 7.4), 5% glycerol, 0.25 mM EDTA pH 8.0, 1 mM PMSF (RPI), 25 μg/ml of lysozyme and DNase I (GoldBio). The suspension was then lysed by sonication and clarified by centrifugation (30,000×g for 0.5 h). Filtered supernatant was loaded into a HisPur cobalt Superflow agarose column (ThermoScientific). The column was washed with 10 column volume (CV) of buffer A, 5 CV of buffer A+5 mM imidazole, and finally eluted with 3 CV of buffer A+100 mM imidazole. The eluate was desalted by dialysis in 150 mM NaCl, 20 mM Tris/HCl, 20 mM imidazole, pH 7.4. SDS-PAGE gel confirmed the expression, solubility, and purity of the peptide. All of our solutions were formulated with ultrapure water (Milli-Q® IQ 7005 water purification system), that has less than 0.29 ng/L calcium ions present. The binding affinity is recorded based on the heat difference between the sample cell and reference cell measured by MicroCal iTC200. Forty successive additions of 1 μl CaCl₂) (2 mM) was added into the sample cell containing 400 μM of the respective purified peptides at one-minute intervals. The cells were insulated by an adiabatic jacket held at 25° C. The heat of dilution of Ca²⁺ was not subtracted from data sets, as the signal was prohibitively small for several of the mutant channels. The exothermic energy of the bimolecular interaction between Ca²⁺ and peptide during each injection was analyzed by integrating the change in heat to generate the binding isotherm. The resulting relationship was fit (Origin 7.0) with a one-site independent binding model to determine the binding affinity (Kd).

Production of the Pkd2^{-X-Z} mouse strains and histology analysis. SgRNAs for the T769A and E772A mutations (GCTCACGCTCGGTCAGTTCC) and the inserted V5 tag (CACGTGTGGATTATTAGGCA) were designed using the CRISPR Design tool (http://crispr.mit.edu/). To edit the mouse genome, single-stranded oligodeoxynucleotide (ssODN) donor templates for T769A:E772A point mutations, and another with V5 tag in-frame at the C-terminus were synthesized (IDT). A sgRNA plasmid was constructed and linearized, followed by in vitro transcription of sgRNAs using MEGAshortscript™ Kit (Invitrogen). The yield and quality of sgRNA was assessed by absorbance ratio and gel electrophoresis (Shen et al., 2014). In vitro transcribed sgRNAs, Cas9 protein (NEB) and two donor ssODN templates were microinjected into zygotes, followed by culture and transfer of blastocysts into the uterus of pseudo-pregnant ICR female mice (Horii et al., 2014; Wang et al., 2013). Founders were identified by PCR amplification of genomic DNA from tail biopsies followed by sequencing of PCR products (Shen et al., 2014). Genotyping of experimental mice is done by allele specific PCR primers (capitalized) for the T769A and E772A double mutant (TCAAGAGCTTGCTGAACGAGC and tgtttaccaaggtcttgggcaagca) and wild type alleles (CCAGGAACTGACCGAGCGTGA and tgtttaccaaggtcttgggcaagca)

Production of the Pkd2^{del-Z} mouse strains, MRI and kidney histology analysis. The Pkd2^{del-Z} mouse was generated using the CRISPR/Cas9 method with the guide RNA 5′AAACAGCGTGAGCATCAACAGATGC3′. To characterize their phenotype Pkd2^{+/+} (10 males, 8 females), Pkd2^{+/del-Z} (10 males, 9 females) and Pkd2^{del-Z/del-Z} (10 males, 8 females) were MRI scanned at 9 and 12 months of age. Nine-month-old cPkd2 mice at (10 males, 8 females) were fed doxycycline through drinking water for one week to genetically attenuate Pkd2 expression (Pax8^{rTA}; TetO-cre; Pkd2^{fl/fl}), as previously described (Liu et al., 2018; Ma et al., 2013). MRI imaging was conducted at Northwestern University's Center for Advanced Molecular Imaging using a Bruker BioSpec (9.4 Tesla). Mice were anesthetized and placed in a chamber containing 3% isoflurane and their respiration was monitored for the duration of the scan (Irazabal et al., 2015). The kidneys from Pkd2^{+/+}, Pkd2^{+/del-Z}, Pkd2^{del-Z/del-Z} and cPkd2 mice (10 mice each) were fixed in 40% perfomaldyhyde for 24 hours, sectioned on a cryostat, mounted on glass coverslips and stained with hematoxylin and eosin. Images of medial sections from both kidneys were analyzed using FIJI (ImageJ) to identify cystoid foramen. Images were processed as black and white, and reverse-negative. Then the images were analyzed using the particle analysis search protocol, where the lower limit threshold for circular foramen was set to 20 μm to identify cystoid foramen in the tissue samples. Pkd2^{del-Z} mice were crossed with our previously established ARL13B-EGFP mouse, so that primary cilia can be visualized from living cells during our cilia electrophysiology recordings described in the next section (Liu et al., 2018). Animals were housed at AALAS certified facilities located Yale University, Northwestern University and the Mayo Clinic. All animal procedures and protocols were approved by each universities perspective Institutional Animal Care and Use Committees (IACUCs).

Cell culture of primary inner medullary collecting duct cells and immortalized cell lines. Primary inner medullary collecting ducts cells (pIMCD) were isolated from WT or Pkd2^{del-Z/del-Z} mice co-expressing the ARL13B-EGFP cilia reporter using the previously described method (Liu et al., 2018). Briefly, inner medullae were removed from the kidney and disassociated using a Dulbecco's phosphate buffered solution (DPBS) containing 2 mg/ml collagenase A and 1 mg/ml hyaluronidase. After mechanical disassociation on ice, medullary cells were cultured in Dulbecco's modified essential medium (DMEM) supplemented with 10% fetal bovine serum (FBS) and 100 units/ml penicillin/100 μg/ml streptomycin. Cilia were patched from cells within 6 days after isolation and within one passage. HEK PKD2^{Null} cell lines were generated using the CRISPR/Cas9 gene editing kit available from Addgene and authenticated using PCR analysis, as previously described (Liu et al., 2018). To generate the stable cell lines expressing C-terminally tagged version of WT and deletion mutants of human polycystin-2, the hPKD2 gene was subcloned into lentiviral pLVX-GFP-N1 (Clontech) vector using the Gibson assembly method. The del-Z deletion in hPKD2-GFP was generated using a modified site-directed mutagenesis protocol. Lentiviral infected cells were selected using culture media containing puromycin (2 μg/ml) and sorted (BD FacsMelody) at 5000 to 10,000 counts per minute to enrich for the transgene expression. Stable cell lines were cultured in DMEM supplemented with 10% FBS and 100 units/ml penicillin, 100 units/ml streptomycin and 1 μg/ml puromycin selection antibiotic. On a monthly basis, mycoplasma testing (MycoProbe, R&D systems) was performed on all active cultures in our incubators.

Electrophysiology. The electrophysiologist was blinded by a third party in the laboratory, where test groups were assigned a letter to conceal the genetic identity of the cells being evaluated. The identity cells was remained unknown by the electrophysiologist until the analysis was complete. Ciliary ion currents were recorded using borosilicate glass electrodes polished to resistances of 14-23 MΩ using the cilium patch method previously described (Vien et al., 2020). Single channel currents measured in the inside-out configuration were recorded in symmetrical sodium concentrations. All of our solutions were formulated with ultrapure water (Milli-Q® IQ 7005 water) which has less than 0.29 ng/L calcium ions present. The internal solution (bath) contained (in mM): 120 NaMES, 10 NaCl, and 10 HEPES. Calcium was buffered with 5 EGTA (ethylene glycol-bis(o-aminoethyl ether)-N,N,N′,N′-tetraacetic acid), 5 Na4-BAPTA (Glycine, N, N′-[1,2-ethanediylbis(oxy-2,1-phenylene)]bis[N-(carboxymethyl)]-,tetrasodium) and 0.5 EDTA (ethylene glycol-bis(O-aminoethyl ether)-N,N,N′,N′-tetraacetic acid); free calcium was calculated using Maxchilator and titration of 1 M CaCl₂) solution (Bers et al., 2010); pH was adjusted to 7.3 using NaOH. Standard external solution (pipette electrode) contained 150 NaCl, 10 HEPES, 2 CaCl₂); pH 7.4. All solutions were osmotically balanced to 300 (±7) mOsm with d-mannitol. Whole cell currents used to measure CDD were also recorded in symmetrical sodium concentration, placing the internal recording solution in the pipette electrode and the external recording solution in the bath. Data were collected using an Axopatch 200B patch clamp amplifier, Digidata 1550B, and pClamp 10 software. Single channel currents were digitized at 50 kHz and low-pass filtered at 10 kHz. Intraciliary conditions were controlled using an Octaflow II rapid perfusion system (ALA systems) in which the patched cilia and electrode were held in the perfusate stream. Data were analyzed by Igor Pro 7.00 (Wavemetrics, Lake Oswego, Oreg.). The polycystin-2 open probability (Po) current-voltage relationships were fit to a Boltzman equation, f(x)=1/(1+exp[V−V_{1/2}]/k), to estimate half-maximal voltage (V_{1/2}) require to open the channels. The potency of calcium opening polycystin-2 channels was estimated by integrating the single channel current measured in response to elevating the internal calcium concentration ([Caᵢₙ]). The average integrated current was fit the hill equation, f(x)=base+(max−base)/{1+(EC₅₀/[Caᵢₙ]) to estimate the effective concentration of calcium (EC₅₀) required to half maximally stimulate the polycystin-2 response.

Intracellular calcium measurements using Fura-2. Vasopressin-mediated intracellular calcium responses were measured from pIMCD cells 24-72 hours after cells were isolated. Prior to seeding the cells, glass bottom dishes were pre-coated with poly-L-lysine and laminin (Sigma). The cells were incubated for 1 hour at 37° C. in complete medium after loading with 2 μg of Fura-2/AM (Invitrogen). The cells were then washed with and stored for 15 minutes in Tyrode's solution (in mM): 140 NaCl, 4 KCl, 2 MgCl2, 2 CaCl₂), 10 Glucose, 10 HEPES. Cells were adjusted in pH to 7.4 with NaOH and osmolarity to 300 mOsm with d-mannitol. Cells were placed under an inverted wide-field microscope equipped with 20× objective lens (Olympus IX81) and the stage temperature was held at 37° C. (Tokai Hit). Images of fura-2 fluorescence remission at 520 nm were captured every 2 seconds during excitation at 380 nm (Ca²⁺ free) and 340 nm (Ca²⁺ bound). Images were acquired and analyzed using SlideBook (Intelligent imaging solutions) which synchronizes the filter wheel changer (Lambda 10-3, Sutter Instrument) and the camera (ImagEMX2, Hamamatsu). In each trial or replicate (N), the emission fluorescence (340:380 nm) ratio of 15 to 30 cells was recorded after subtracting the background fluorescence levels. This ratio was averaged at the start (for resting level) and the response was reported after extracellular vasopressin or carbachol treatment (for maximal response). At least three replicates (N) were performed from cells isolated from five animals of the same genotype.

Statistical analysis. Sample sizes were determined based on 2-sample t test power analysis of the recorded variance from pilot study results from each assay, where target power=0.9 and α=0.05. Statistical comparisons were made using one-way ANOVA or two-tailed Student's t-tests using OriginPro software (OriginLab, Northampton Mass.) or Excel (Microsoft). Experimental values are reported as the mean±S.D. unless otherwise stated. Differences in mean values were considered significant at P<0.05. All of our results are normally distributed per Shapiro-Wilk Test.

Reagent and data availability statement: cDNA constructs, and mouse constructs used in this study will be available upon request by PGD, SS or PH unless the item(s) are already deposited and available through addgene (https://www.addgene.org/). Data files associated with this manuscript are available through Northwestern University's institutional repository service (ARCH): https://doi.org/10.21988/n2-yhyk-9937

### REFERENCES

Allen, M. D., S. Qamar, M. K. Vadivelu, R. N. Sandford, and M. Bycroft. 2014. A high-resolution structure of the EF-hand domain of human polycystin-2. Protein Sci. 23:1301-1308.
Ben-Johny, M., and D. T. Yue. 2014. Calmodulin regulation (calmodulation) of voltage-gated calcium channels. J Gen Physiol. 143:679-692.
Bers, D. M., C. W. Patton, and R. Nuccitelli. 2010. A practical guide to the preparation of Ca(2+) buffers. Methods Cell Biol. 99:1-26.
Brasier, J. L., and E. P. Henske. 1997. Loss of the polycystic kidney disease (PKD1) region of chromosome 16p13 in renal cyst cells supports a loss-of-function model for cyst pathogenesis. J Clin Invest. 99:194-199.
Braun, D. A., and F. Hildebrandt. 2017. Ciliopathies. Cold Spring Harb Perspect Biol. 9.
Celic, A., E. T. Petri, B. Demeler, B. E. Ehrlich, and T. J. Boggon. 2008. Domain mapping of the polycystin-2 C-terminal tail using de novo molecular modeling and biophysical analysis. J Biol Chem. 283:28305-28312.
Celic, A. S., E. T. Petri, J. Benbow, M. E. Hodsdon, B. E. Ehrlich, and T. J. Boggon. 2012. Calcium-induced conformational changes in C-terminal tail of polycystin-2 are necessary for channel gating. J Biol Chem. 287:17232-17240.
Chagot, B., F. Potet, J. R. Balser, and W. J. Chazin. 2009. Solution NMR structure of the C-terminal EF-hand domain of human cardiac sodium channel NaV1.5. J Biol Chem. 284:6436-6445.
DeCaen, P. G., M. Delling, T. N. Vien, and D. E. Clapham. 2013. Direct recording and molecular identification of the calcium channel of primary cilia. Nature. 504:315-318.
DeCaen, P. G., X. Liu, S. Abiria, and D. E. Clapham. 2016. Atypical calcium regulation of the PKD2-L1 polycystin ion channel. Elife. 5.
Delling, M., P. G. DeCaen, J. F. Doerner, S. Febvay, and D. E. Clapham. 2013. Primary cilia are specialized calcium signalling organelles. Nature. 504:311-314.
DeMaria, C. D., T. W. Soong, B. A. Alseikhan, R. S. Alvania, and D. T. Yue. 2001. Calmodulin bifurcates the local Ca2+ signal that modulates P/Q-type Ca2+ channels. Nature. 411:484-489.
Fanger, C. M., S. Ghanshani, N. J. Logsdon, H. Rauer, K. Kalman, J. Zhou, K. Beckingham, K. G. Chandy, M. D. Cahalan, and J. Aiyar. 1999. Calmodulin mediates calcium-dependent activation of the intermediate conductance KCa channel, IKCal. J Biol Chem. 274:5746-5754.
Gardill, B. R., R. E. Rivera-Acevedo, C. C. Tung, M. Okon, L. P. McIntosh, and F. Van Petegem. 2018. The voltage-gated sodium channel EF-hands form an interaction with the III-IV linker that is disturbed by disease-causing mutations. Sci Rep. 8:4483.
Grantham, J. J. 2001. Polycystic kidney disease: from the bedside to the gene and back. Curr Opin Nephrol Hypertens. 10:533-542.
Grieben, M., A. C. Pike, C. A. Shintre, E. Venturi, S. El-Ajouz, A. Tessitore, L. Shrestha, S. Mukhopadhyay, P. Mahajan, R. Chalk, N. A. Burgess-Brown, R. Sitsapesan, J. T. Huiskonen, and E. P. Carpenter. 2017. Structure of the polycystic kidney disease TRP channel Polycystin-2 (PC2). Nat Struct Mol Biol. 24:114-122.
Guo, W., B. Sun, Z. Xiao, Y. Liu, Y. Wang, L. Zhang, R. Wang, and S. R. Chen. 2016. The EF-hand Ca2+ Binding Domain Is Not Required for Cytosolic Ca2+ Activation of the Cardiac Ryanodine Receptor. J Biol Chem. 291:2150-2160.
Happe, H., and D. J. Peters. 2014. Translational research in ADPKD: lessons from animal models. Nat Rev Nephrol. 10:587-601.
Hildebrandt, F., T. Benzing, and N. Katsanis. 2011. Ciliopathies. N Engl J Med. 364:1533-1543.
Hildebrandt, F., and W. Zhou. 2007. Nephronophthisis-associated ciliopathies. J Am Soc Nephrol. 18:1855-1871.
Horii, T., Y. Arai, M. Yamazaki, S. Morita, M. Kimura, M. Itoh, Y. Abe, and I. Hatada. 2014. Validation of microinjection methods for generating knockout mice by CRISPR/Cas-mediated genome engineering. Sci Rep. 4:4513.
Hughes, J., C. J. Ward, B. Peral, R. Aspinwall, K. Clark, J. L. San Millan, V. Gamble, and P. C. Harris. 1995. The polycystic kidney disease 1 (PKD1) gene encodes a novel protein with multiple cell recognition domains. Nat Genet. 10:151-160.
Irazabal, M. V., P. K. Mishra, V. E. Torres, and S. I. Macura. 2015. Use of Ultra-high Field MRI in Small Rodent Models of Polycystic Kidney Disease for In Vivo Phenotyping and Drug Monitoring. J Vis Exp:e52757.
Johnson, C. N., F. Potet, M. K. Thompson, B. M. Kroncke, A. M. Glazer, M. W. Voehler, B. C. Knollmann, A. L. George, Jr., and W. J. Chazin. 2018. A Mechanism of Calmodulin Modulation of the Human Cardiac Sodium Channel. Structure. 26:683-694 e683.
Kleene, N. K., and S. J. Kleene. 2012. A method for measuring electrical signals in a primary cilium. Cilia. 1.
Kleene, S. J., and N. K. Kleene. 2017. The native TRPP2-dependent channel of murine renal primary cilia. Am J Physiol Renal Physiol. 312:F96-F108.
Koptides, M., C. Hadjimichael, P. Koupepidou, A. Pierides, and C. Constantinou Deltas. 1999. Germinal and somatic mutations in the PKD2 gene of renal cysts in autosomal dominant polycystic kidney disease. Hum Mol Genet. 8:509-513.
Koulen, P., Y. Cai, L. Geng, Y. Maeda, S. Nishimura, R. Witzgall, B. E. Ehrlich, and S. Somlo. 2002. Polycystin-2 is an intracellular calcium release channel. Nat Cell Biol. 4:191-197.
Kuo, I. Y., A. L. Brill, F. O. Lemos, J. Y. Jiang, J. L. Falcone, E. P. Kimmerling, Y. Cai, K. Dong, D. L. Kaplan, D. P. Wallace, A. M. Hofer, and B. E. Ehrlich. 2019. Polycystin 2 regulates mitochondrial Ca(2+) signaling, bioenergetics, and dynamics through mitofusin 2. Sci Signal. 12.
Lee, A., S. T. Wong, D. Gallagher, B. Li, D. R. Storm, T. Scheuer, and W. A. Catterall. 1999. Ca2+/calmodulin binds to and modulates P/Q-type calcium channels. Nature. 399:155-159.
Lee, C. H., and R. MacKinnon. 2018. Activation mechanism of a human SK-calmodulin channel complex elucidated by cryo-EM structures. Science. 360:508-513.
Liu, X., T. Vien, J. Duan, S. H. Sheu, P. G. DeCaen, and D. E. Clapham. 2018. Polycystin-2 is an essential ion channel subunit in the primary cilium of the renal collecting duct epithelium. Elife. 7.
Ma, M., X. Tian, P. Igarashi, G. J. Pazour, and S. Somlo. 2013. Loss of cilia suppresses cyst growth in genetic models of autosomal dominant polycystic kidney disease. Nat Genet. 45:1004-1012.
Menezes, L. F., and G. G. Germino. 2013. Murine Models of Polycystic Kidney Disease. Drug Discov Today Dis Mech. 10:e153-e158.
Mizuno, K., K. Shiozawa, T. A. Katoh, K. Minegishi, T. Ide, Y. Ikawa, H. Nishimura, K. Takaoka, T. Itabashi, A. H. Iwane, J. Nakai, H. Shiratori, and H. Hamada. 2020. Role of Ca(2+) transients at the node of the mouse embryo in breaking of left-right symmetry. Sci Adv. 6:eaba1195.
Mochizuki, T., G. Wu, T. Hayashi, S. L. Xenophontos, B. Veldhuisen, J. J. Saris, D. M. Reynolds, Y. Cai, P. A. Gabow, A. Pierides, W. J. Kimberling, M. H. Breuning, C. C. Deltas, D. J. Peters, and S. Somlo. 1996. PKD2, a gene for polycystic kidney disease that encodes an integral membrane protein. Science. 272:1339-1342.
Morick, D., M. Schatz, R. Hubrich, H. Hoffmeister, A. Krefft, R. Witzgall, and C. Steinem. 2013. Phosphorylation of C-terminal polycystin-2 influences the interaction with PIGEA14: a QCM study based on solid supported membranes. Biochem Biophys Res Commun. 437:532-537.
Nachury, M. V., and D. U. Mick. 2019. Establishing and regulating the composition of cilia for signal transduction. Nat Rev Mol Cell Biol. 20:389-405.
Nejatbakhsh, N., and Z. P. Feng. 2011. Calcium binding protein-mediated regulation of voltage-gated calcium channels linked to human diseases. Acta Pharmacol Sin. 32:741-748.
Ostrowski, L. E., K. Blackburn, K. M. Radde, M. B. Moyer, D. M. Schlatzer, A. Moseley, and R. C. Boucher. 2002. A proteomic analysis of human cilia: identification of novel components. Mol Cell Proteomics. 1:451-465.
Park, E. Y. J., J. Y. Baik, M. Kwak, and I. So. 2019. The role of calmodulin in regulating calcium-permeable PKD2L1 channel activity. Korean J Physiol Pharmacol. 23:219-227.
Pazour, G. J., N. Agrin, J. Leszyk, and G. B. Witman. 2005. Proteomic analysis of a eukaryotic cilium. J Cell Biol. 170:103-113.
Pei, Y. 2001. A “two-hit” model of cystogenesis in autosomal dominant polycystic kidney disease? Trends Mol Med. 7:151-156.
Petri, E. T., A. Celic, S. D. Kennedy, B. E. Ehrlich, T. J. Boggon, and M. E. Hodsdon. 2010. Structure of the EF-hand domain of polycystin-2 suggests a mechanism for Ca2+-dependent regulation of polycystin-2 channel activity. Proc Natl Acad Sci USA. 107:9176-9181.
Qian, F., F. J. Germino, Y. Cai, X. Zhang, S. Somlo, and G. G. Germino. 1997. PKD1 interacts with PKD2 through a probable coiled-coil domain. Nat Genet. 16:179-183.
Qian, Q., L. W. Hunter, M. Li, M. Marin-Padilla, Y. S. Prakash, S. Somlo, P. C. Harris, V. E. Torres, and G. C. Sieck. 2003. Pkd2 haploinsufficiency alters intracellular calcium regulation in vascular smooth muscle cells. Hum Mol Genet. 12:1875-1880.
Sammels, E., B. Devogelaere, D. Mekahli, G. Bultynck, L. Missiaen, J. B. Parys, Y. Cai, S. Somlo, and H. De Smedt. 2010. Polycystin-2 activation by inositol 1,4,5-trisphosphate-induced Ca2+ release requires its direct association with the inositol 1,4,5-trisphosphate receptor in a signaling microdomain. J Biol Chem. 285:18794-18805.
Santoso, N. G., L. Cebotaru, and W. B. Guggino. 2011. Polycystin-1, 2, and STIM1 interact with IP(3)R to modulate ER Ca release through the PI3K/Akt pathway. Cell Physiol Biochem. 27:715-726.
Schindelin, J., I. Arganda-Carreras, E. Frise, V. Kaynig, M. Longair, T. Pietzsch, S. Preibisch, C. Rueden, S. Saalfeld, B. Schmid, J. Y. Tinevez, D. J. White, V. Hartenstein, K. Eliceiri, P. Tomancak, and A. Cardona. 2012. Fiji: an open-source platform for biological-image analysis. Nat Methods. 9:676-682.
Shah, V. N., T. L. Wingo, K. L. Weiss, C. K. Williams, J. R. Balser, and W. J. Chazin. 2006. Calcium-dependent regulation of the voltage-gated sodium channel hH1: intrinsic and extrinsic sensors use a common molecular switch. Proc Natl Acad Sci USA. 103:3592-3597.
Shen, B., W. Zhang, J. Zhang, J. Zhou, J. Wang, L. Chen, L. Wang, A. Hodgkins, V. Iyer, X. Huang, and W. C. Skarnes. 2014. Efficient genome modification by CRISPR-Cas9 nickase with minimal off-target effects. Nat Methods. 11:399-402.
Shen, P. S., X. Yang, P. G. DeCaen, X. Liu, D. Bulkley, D. E. Clapham, and E. Cao. 2016. The Structure of the Polycystic Kidney Disease Channel PKD2 in Lipid Nanodiscs. Cell. 167:763-773 e711.
Streets, A. J., A. J. Needham, S. K. Gill, and A. C. Ong. 2010. Protein kinase D-mediated phosphorylation of polycystin-2 (TRPP2) is essential for its effects on cell growth and calcium channel activity. Mol Biol Cell. 21:3853-3865.
Su, Q., F. Hu, X. Ge, J. Lei, S. Yu, T. Wang, Q. Zhou, C. Mei, and Y. Shi. 2018. Structure of the human PKD1-PKD2 complex. Science. 361.
Tran, N. T., T. Sommermann, R. Graf, J. Trombke, J. Pempe, K. Petsch, R. Kuhn, K. Rajewsky, and V. T. Chu. 2019. Efficient CRISPR/Cas9-Mediated Gene Knockin in Mouse Hematopoietic Stem and Progenitor Cells. Cell Rep. 28:3510-3522 e3515.
Vassilev, P. M., L. Guo, X. Z. Chen, Y. Segal, J. B. Peng, N. Basora, H. Babakhanlou, G. Cruger, M. Kanazirska, C. Ye, E. M. Brown, M. A. Hediger, and J. Zhou. 2001. Polycystin-2 is a novel cation channel implicated in defective intracellular Ca(2+) homeostasis in polycystic kidney disease. Biochem Biophys Res Commun. 282:341-350.
Venkatachalam, K., and C. Montell. 2007. TRP channels. Annu Rev Biochem. 76:387-417.
Vien, T. N., J. Wang, L. C. T. Ng, E. Cao, and P. G. DeCaen. 2020. Molecular dysregulation of ciliary polycystin-2 channels caused by variants in the TOP domain. Proc Natl Acad Sci USA.
Wang, C., B. C. Chung, H. Yan, H. G. Wang, S. Y. Lee, and G. S. Pitt. 2014. Structural analyses of Ca(2)(+)/CaM interaction with NaV channel C-termini reveal mechanisms of calcium-dependent regulation. Nat Commun. 5:4896.
Wang, H., H. Yang, C. S. Shivalila, M. M. Dawlaty, A. W. Cheng, F. Zhang, and R. Jaenisch. 2013. One-step generation of mice carrying mutations in multiple genes by CRISPR/Cas-mediated genome engineering. Cell. 153:910-918.
West, J. W., D. E. Patton, T. Scheuer, Y. Wang, A. L. Goldin, and W. A. Catterall. 1992. A cluster of hydrophobic amino acid residues required for fast Na(+)-channel inactivation. Proc Natl Acad Sci USA. 89:10910-10914.
Wilkes, M., M. G. Madej, L. Kreuter, D. Rhinow, V. Heinz, S. De Sanctis, S. Ruppel, R. M. Richter, F. Joos, M. Grieben, A. C. Pike, J. T. Huiskonen, E. P. Carpenter, W. Kuhlbrandt, R. Witzgall, and C. Ziegler. 2017. Molecular insights into lipid-assisted Ca(2+) regulation of the TRP channel Polycystin-2. Nat Struct Mol Biol. 24:123-130.
Wilson, P. D. 2008. Mouse models of polycystic kidney disease. Curr Top Dev Biol. 84:311-350.
Wingo, T. L., V. N. Shah, M. E. Anderson, T. P. Lybrand, W. J. Chazin, and J. R. Balser. 2004. An EF-hand in the sodium channel couples intracellular calcium to cardiac excitability. Nat Struct Mol Biol. 11:219-225.
Wu, G., V. D'Agati, Y. Cai, G. Markowitz, J. H. Park, D. M. Reynolds, Y. Maeda, T. C. Le, H. Hou, Jr., R. Kucherlapati, W. Edelmann, and S. Somlo. 1998. Somatic inactivation of Pkd2 results in polycystic kidney disease. Cell. 93:177-188.
Wu, G., G. S. Markowitz, L. Li, V. D. D'Agati, S. M. Factor, L. Geng, S. Tibara, J. Tuchman, Y. Cai, J. H. Park, J. van Adelsberg, H. Hou, Jr., R. Kucherlapati, W. Edelmann, and S. Somlo. 2000. Cardiac defects and renal failure in mice with targeted mutations in Pkd2. Nat Genet. 24:75-78.
Xia, X. M., X. Zeng, and C. J. Lingle. 2002. Multiple regulatory sites in large-conductance calcium-activated potassium channels. Nature. 418:880-884.
Yang, Y., and B. E. Ehrlich. 2016. Structural studies of the C-terminal tail of polycystin-2 (PC2) reveal insights into the mechanisms used for the functional regulation of PC2. J Physiol. 594:4141-4149.
Yang, Y., C. Keeler, I. Y. Kuo, E. J. Lolis, B. E. Ehrlich, and M. E. Hodsdon. 2015. Oligomerization of the polycystin-2 C-terminal tail and effects on its Ca²⁺-binding properties. J Biol Chem. 290:10544-10554.
Yu, Y., M. H. Ulbrich, M. H. Li, Z. Buraei, X. Z. Chen, A. C. Ong, L. Tong, E. Y. Isacoff, and J. Yang. 2009. Structural and molecular basis of the assembly of the TRPP2/PKD1 complex. Proc Natl Acad Sci USA. 106:11558-11563.
Zhou, J., R. Olcese, N. Qin, F. Noceti, L. Birnbaumer, and E. Stefani. 1997. Feedback inhibition of Ca2+ channels by Ca2+ depends on a short sequence of the C terminus that does not include the Ca2+-binding function of a motif with similarity to Ca2+-binding domains. Proc Natl Acad Sci USA. 94:2301-2305.
Zhu, J., Y. Yu, M. H. Ulbrich, M. H. Li, E. Y. Isacoff, B. Honig, and J. Yang. 2011. Structural model of the TRPP2/PKD1 C-terminal coiled-coil complex produced by a combined computational and experimental approach. Proc Natl Acad Sci USA. 108:10133-10138.

### Example 3—Identification of Pharmacological Activators of PKD2

Abstract

Though approximately ninety-five percent of cases of ADPKD are associated with variants in the polycystin genes, PKD1 or PKD2, which encode for polycystin-1 and 2, the molecular mechanism by which mutations in PKD1 or PKD2 cause disease are poorly understood. In Example 1, we discovered that mutations in the TOP domain of PKD2 do not interrupt the normal trafficking of PKD2 to the primary cilium. However, mutations in the TOP domain do disrupt the ion channel function of PKD2. Current pharmacological approaches to treating autosomal dominant polycystic kidney disease (ADPKD) are limited to competitive vasopressin receptor 2 antagonism by the drug tolvaptan which has been shown to delay the need for kidney transplant, but does not treat the underlying cause of ADPKD which is mutation in PKD1 or PKD2. Therefore, we hypothesized that screening for activators of PKD2 function could present a possible treatment for the root-cause of ADPKD.

Introduction

Autosomal dominant polycystic kidney disease (ADPKD) impacts 1:1000 individuals world-wide, which translates to ˜12.5 million people. Variants in the polycystin genes PKD1 and PKD2 cause ADPKD, although it is still unknown how these variants impact polycystin-1 (PKD1) and polycystin-2 (PKD2), which are the proteins encoded by PKD1 and PKD2, respectively. PKD1 and PKD2 localize to the primary cilia of collecting ducts of the kidneys, where they interact and form receptor-ion channels. Approximately 80% of ADPKD variants are located in PKD1, and approximately 20% of ADPKD variants are located in PKD2.

Each PKD2 channel contains 4 subunits comprising 1-986 amino acids. (See Shen et al., Cell 2016). Each subunit contains 4 domains which include the TOP domain, the Voltage Sensor Domain, the Pore Domain, and the C-Terminus Domain. The TOP domain is the site of most ADPKD variants in PKD2, where ˜78% of ADPKD variants are located in the TOP domain. Some of the most pathogenic variants are located with the Finger 1 motif of the TOP domain and include C331S, R322Q, K322W, R325Q, and K325P, which are suggested to disrupt discrete chemical interactions within the TOP domain. (See Vien et al. PNAS, 2020 and Example 1). Under two hypotheses: 1) the variants are suggested to alter channel assembly and disrupt ciliary localization; and/or 2) the variants are suggested to disrupt ion channel function.

The Finger 1 motif variant, C331S variant and other Finger 1 motif variants that include R322W, R322Q, R325P, and R325Q, are not observed to alter ciliary localization. (See Vien et al. PNAS, 2020 and Example 1). However, although C331S is observed to form tetrameric channels, the overall TOP domain structure is observed to be disordered/destabilized. (See Vien et al. PNAS, 2020 and Example 1). Furthermore, the C331S, R322W, R322Q, R325P, and R325Q variants are observed to inhibit channel activity of PKD2 in the cilia. (See Vien et al. PNAS, 2020 and Example 1). In particular, the C331S, R322W, R322Q, R325P, and R325Q variants cause a depolarizing shift in voltage dependence. (See Vien et al. PNAS, 2020 and Example 1).

Methods and Results

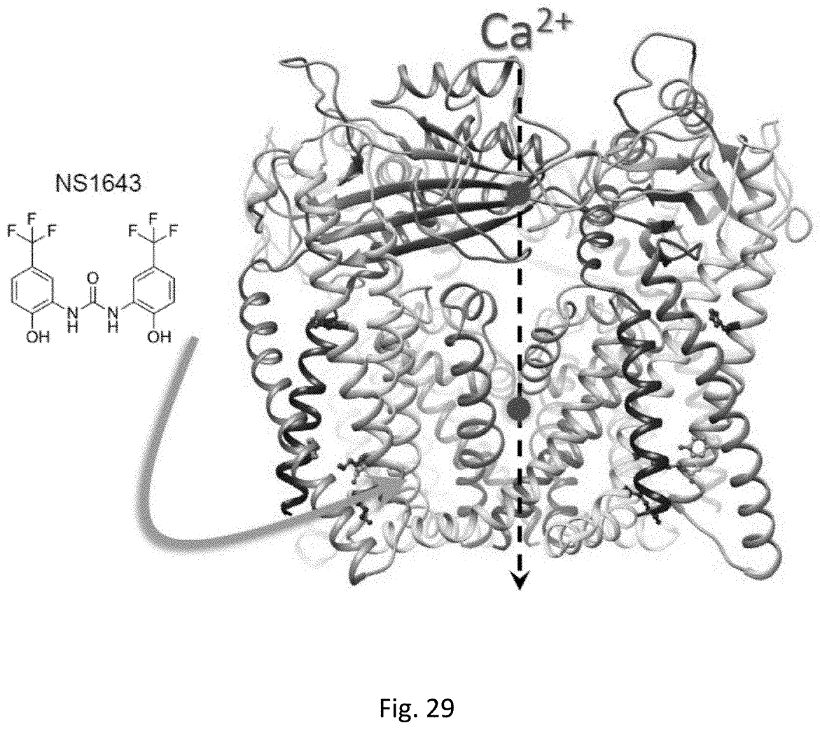

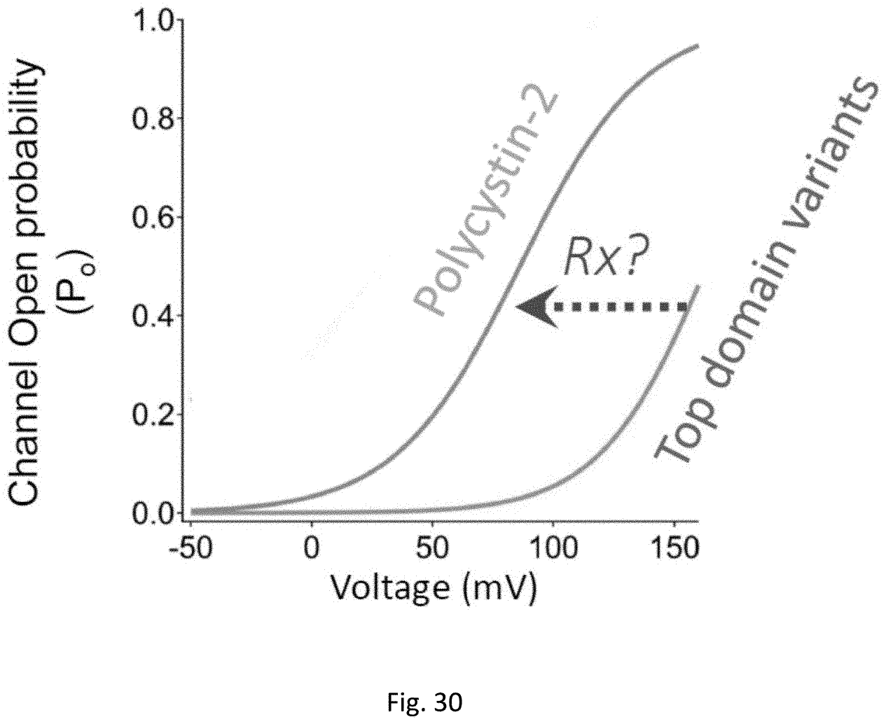

Based on the observed depolarizing shift in voltage dependence for the TOP Domain variants, we devised a screening strategy for channel activity in order to identify compounds that can activate channel activity. (See FIG. 29 and FIG. 30). Our strategy utilized kidney cyst cells derived from ADPKD patients that express PKD2 variants. Our strategy also utilized high-resolution cryo-EM to structurally define small molecule receptor sites within PKD2.

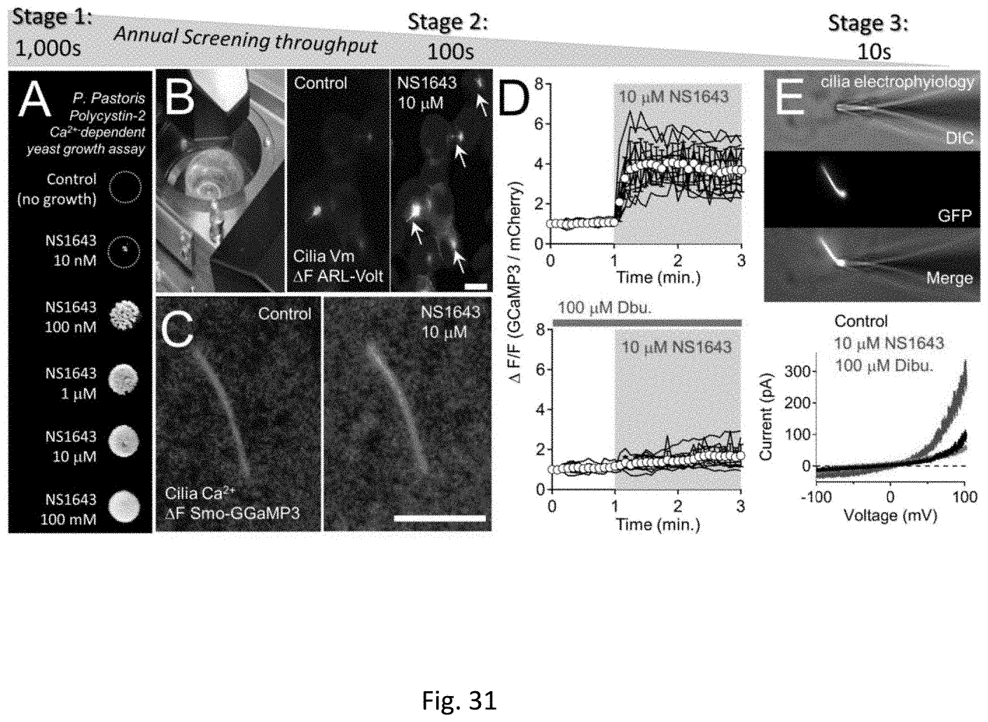

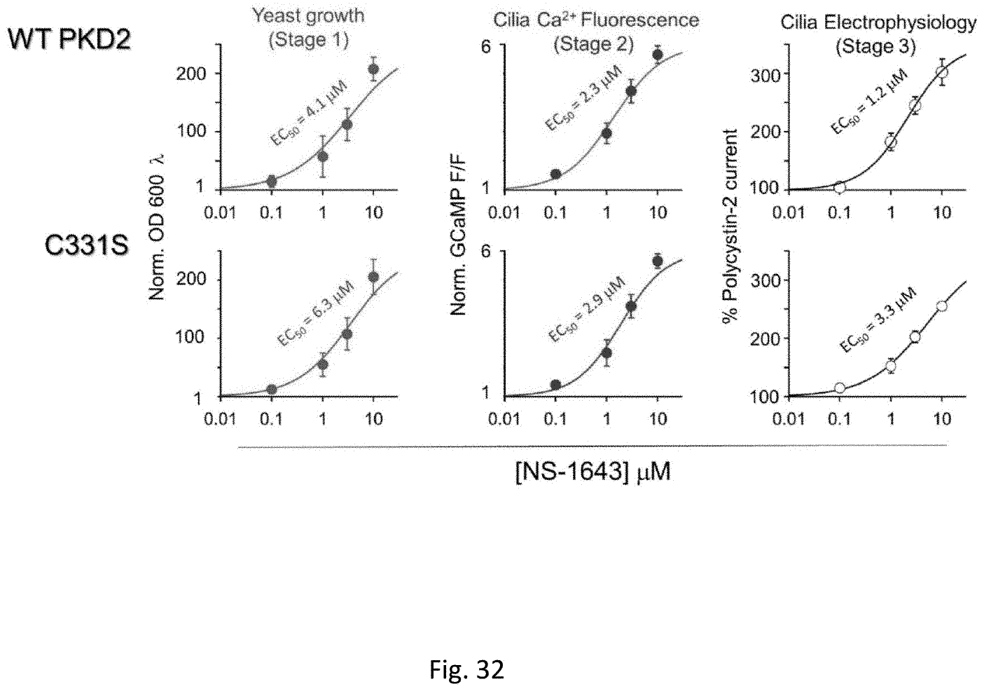

Our screening strategy included three stages. (See FIG. 31). In Stage 1 of our screening assay, we utilized cyst cells in a P. pastoris (strain YS634) PKD2 Ca²⁺-dependent yeast growth assay. P. pastoris growth is dependent on induction and stimulation of PKD2 ion transport by test compounds including NS1643, calmidazolium, and W-13. In Stage 2, we measured PKD2 activation by the test compounds using increased ciliary Ca2+ fluorescence and membrane potential from human kidney collecting duct cells in which PKD2 had been knocked out by CRISPR-Cas9. In Stage 3, we assessed activation of polystin-2 dependent currents by the test compounds via imaging of patched cilia from collecting ducts and measuring change in current versus voltage. The test compound NS1643 was identified as an activator of PKD2 activity. (See FIG. 32).

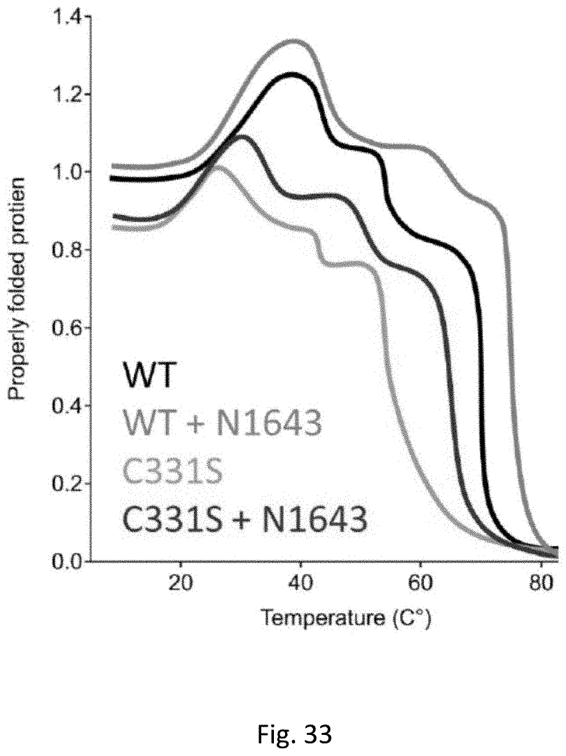

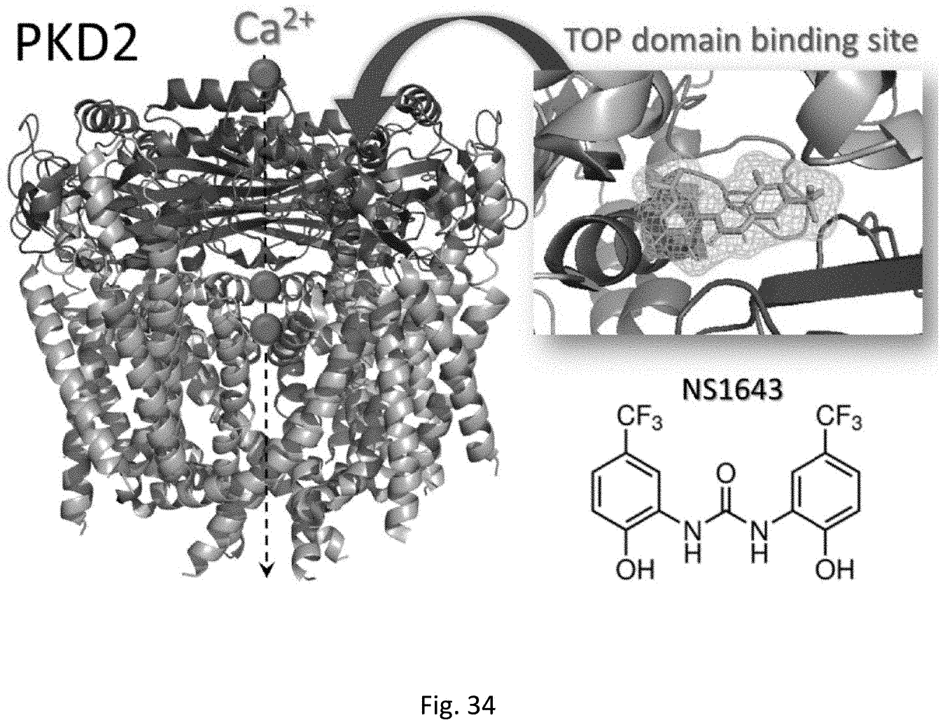

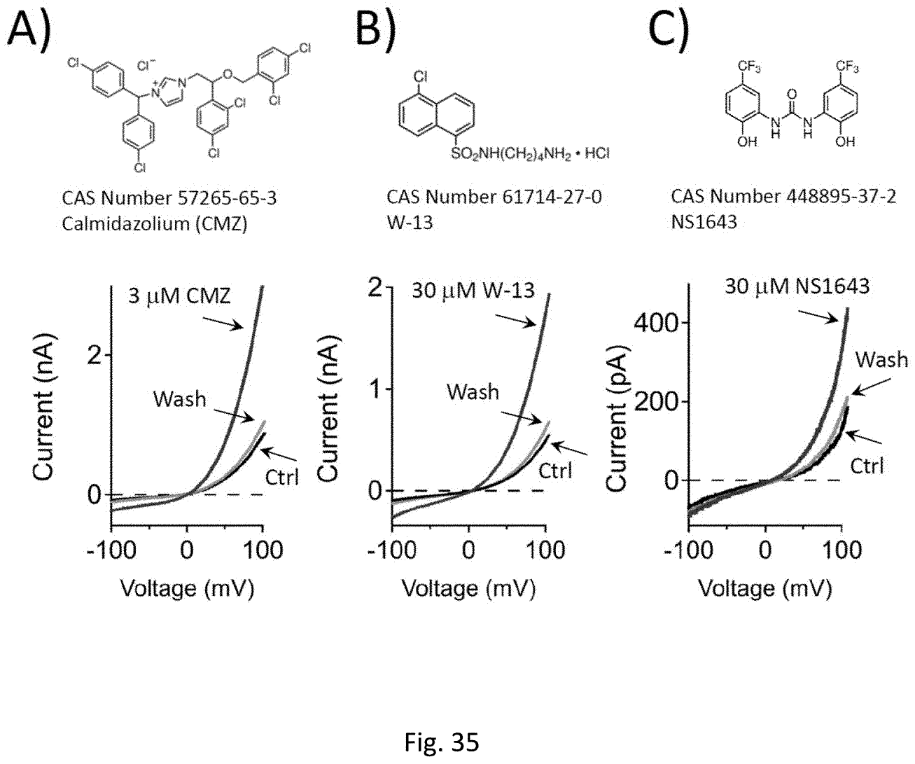

We tested the thermal stability of PKD2 and the C331S variant in the presence or absence of NS1643. (See FIG. 33). We also performed Rosetta modeling. (See FIG. 35). Our results suggest that NS1643 binds to the TOP domain of PKD2 and stabilizes the structure of PKD2.

We also tested the additional compound CAS Number 57265-65-3 (calmidazolium (CMZ)), the compound CAS Number 617-27-0 (W-13), and NS1643 in a PKD2 conductance assay. We observed that all of the tested compounds increased PKD2 conductance.

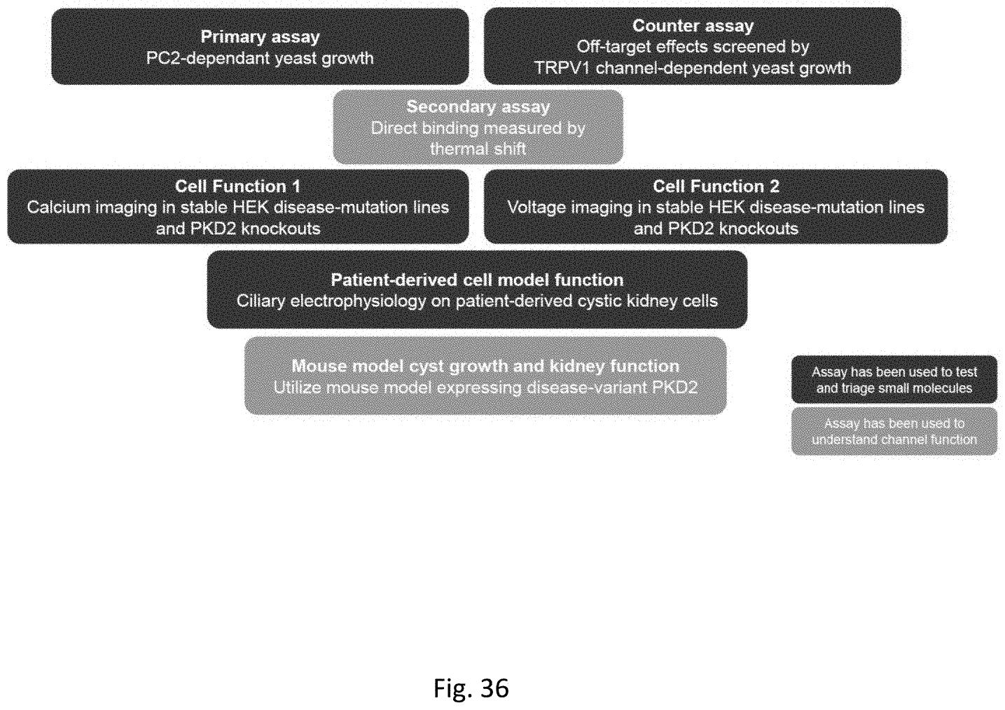

An overview of our screening methods and future work are illustrated in FIG. 36.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method of treating a subject in need of treatment for a disease or disorder associated with polycystin 2 (PDK2) activity, the method comprising administering to the subject an effective amount of a therapeutic agent that activates biological activity of PKD2.

2. The method of claim 1, wherein the disease is a kidney disease.

3. The method of claim 1, wherein the disease is polycystic kidney disease.

4. The method of claim 1, wherein the disease is autosomal dominant polycystic kidney disease (ADPKD).

5. The method of claim 1, wherein the disease is autosomal dominant polycystic kidney disease (ADPKD) characterized by a mutation selected from C331S, R322Q, R322W, R325Q, R325P, and combinations thereof.

6. The method of claim 1, wherein the therapeutic agent activates channel activity of PKD2.

7. The method of claim 1, wherein the therapeutic agent is selected from the group consisting of 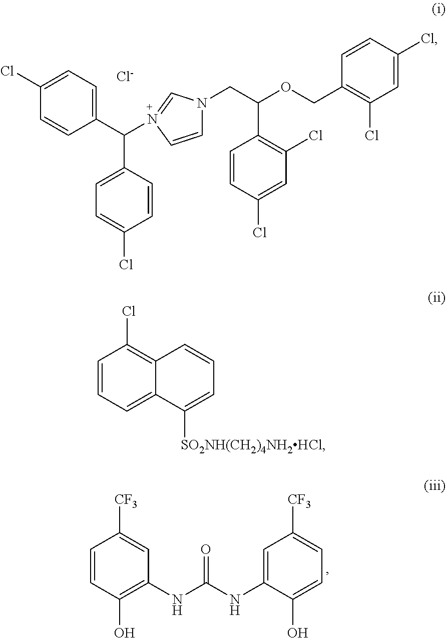
and pharmaceutical salts thereof.

8. The method of claim 1, wherein the therapeutic agent is selected from the group consisting of:
(i) 1-[bis(4-chlorophenyl)methyl]-3-[2,4-dichloro-3-(2,4-dichlorobenzyloxy) phenethyl]imidazolium (or 1-[bis(4-chlorophenyl)methyl]-3-[2-(2,4-dichlorophenyl)-2-(2,4-dichlorobenzyloxy) ethyl]-1H-imidazolium);
(ii) N-(6-Aminohexyl)-5-chloro-1-naphthalenesulfonamide;
(iii) 1,3-bis[2-hydroxy-5-(trifluoromethyl)phenyl]urea; and pharmaceutical salts thereof.

9. The method of claim 1, wherein the therapeutic agent is:

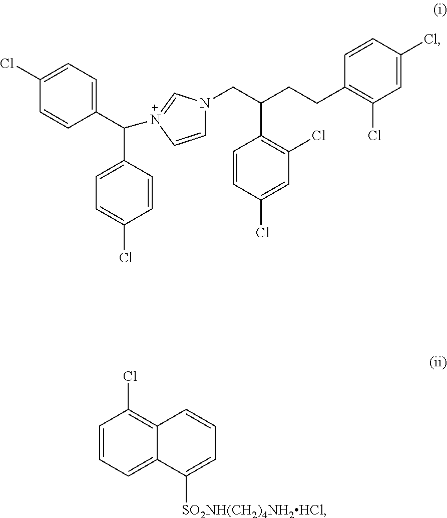

10. The method of claim 1, wherein the therapeutic agent is 1,3-bis[2-hydroxy-5-(trifluoromethyl)phenyl]urea, or a pharmaceutical salt thereof.

11. A pharmaceutical composition comprising: (a) a therapeutic agent that activates biological activity of PKD2; and (b) a suitable pharmaceutical carrier.

12. The pharmaceutical composition of claim 11, wherein the therapeutic agent activates channel activity of PKD2.

13. The pharmaceutical composition of claim wherein the therapeutic agent is selected from the group consisting of 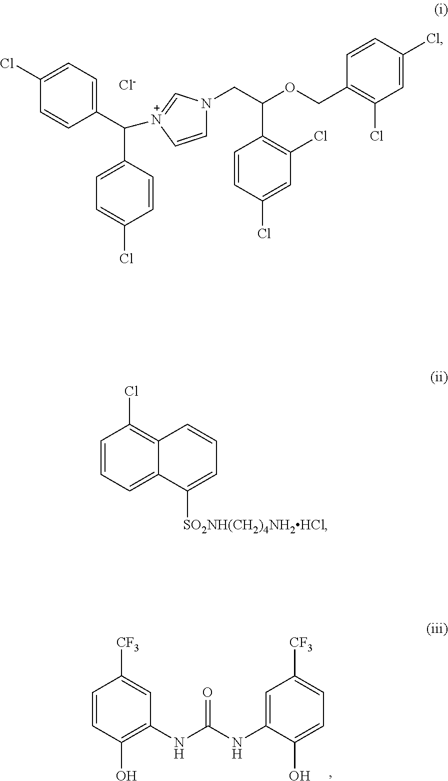
and pharmaceutical salts thereof.

14. The pharmaceutical composition of claim 11, wherein the therapeutic agent is:

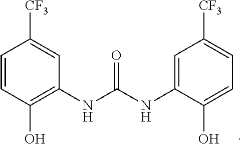 [SMILES: O=C(Nc1cc(C(F)(F)F)ccc1O)Nc1cc(C(F)(F)F)ccc1O]

15. The pharmaceutical composition of claim 11, wherein the therapeutic agent is selected from:
(i) 1-[bis(4-chlorophenyl)methyl]-3-[2,4-dichloro-3-(2,4-dichlorobenzyloxy) phenethyl]imidazolium (or 1-[bis(4-chlorophenyl)methyl]-3-[2-(2,4-dichlorophenyl)-2-(2,4-dichlorobenzyloxy) ethyl]-1H-imidazolium);
(ii) N-(6-Aminohexyl)-5-chloro-1-naphthalenesulfonamide;
(iii) 1,3-bis[2-hydroxy-5-(trifluoromethyl)phenyl]urea; and pharmaceutical salts thereof.

16. The pharmaceutical composition of claim 11, wherein the therapeutic agent is 1,3-bis[2-hydroxy-5-(trifluoromethyl)phenyl]urea, or a pharmaceutical salt thereof.

17. The pharmaceutical composition of claim 11, wherein the composition comprises an effective amount of the therapeutic agent for activating biological activity of PDK2 when administered to a subject in need thereof.

18. A method for identifying an agent that activates the activity of polycystin-2, the method comprising contacting polycystin-2 with the agent and measuring increased activity of polycystin-2 when polycystin-2 is contacted with the agent.

19. The method of claim 18, wherein the increased activity of polycystin-2 comprises increased channel activity.

20. The method of claim 18, wherein the increased activity of polycystin-2 comprises increased transport of a cation selected from Ca²⁺, Na⁺, K⁺, and combinations thereof.


SEQUENCE LISTING (5 sequences)

SEQ ID NO: 1 - protein, 968 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 2 - protein, 483 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAAFICSSYGD

SEQ ID NO: 3 - protein, 912 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 4 - protein, 646 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIICSWRSSMIRTLK

SEQ ID NO: 5 - protein, 874 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV